(19) United States
(12) Patent Application Publication
(10) Pub. No.: US 2023/0235000 A1
(43) Pub. Date: Jul. 27, 2023

(54) TREATMENT OF NEURODEGENERATIVE PROTEINOPATHIES USING FAS APOPTOSIS INHIBITORY MOLECULE (FAIM) OR A FRAGMENT AND/OR A MIMETIC THEREOF

(71) Applicants: Hiroaki KAKU, Portage, MI (US); Thomas L. ROTHSTEIN, Kalamazoo, MI (US)

(72) Inventors: Hiroaki KAKU, Portage, MI (US); Thomas L. ROTHSTEIN, Kalamazoo, MI (US)

(21) Appl. No.: 17/857,797

(22) Filed: Jul. 5, 2022

Related U.S. Application Data
(63) Continuation of application No. PCT/US2021/014778, filed on Jan. 22, 2021.
(60) Provisional application No. 62/965,502, filed on Jan. 24, 2020.

(51) Int. Cl. C07K 14/47; A61K 39/395; A61K 38/17; A61P 25/28

(52) U.S. Cl. CPC C07K 14/4747; A61K 38/1761; A61K 39/3955; A61P 25/28

(57) ABSTRACT

The present technology is directed to fragments of Fas Apoptosis Inhibitory Molecule (FAIM) or mimetics thereof, compositions containing FAIM or fragments and/or mimetics thereof, and methods of treatment and systems comprising FAIM or fragments and/or mimetics thereof. The methods of treatment include treating neurodegenerative neurodegenerative or other proteinopathy such as Alzheimer’s disease, Parkinson’s disease, Huntington’s disease, amyotropic lateral sclerosis, multiple tauopathies, spongiform encephalopathies, familial amyloidotic polyneuropathy, chronic traumatic encephalopathy, or a combination of two or more thereof.

### CROSS REFERENCE TO RELATED APPLICATIONS

The present application is a bypass continuation of PCT Application No. PCT/US2021/014778, filed on Jan. 22, 2021, which claims the benefit of U.S. Provisional Pat. Application No. 62/965,502, filed on Jan. 24, 2020. The contents of these applications are hereby incorporated by reference in their entireties.

### SEQUENCE LISTING

The instant application contains a Sequence Listing which has been submitted electronically in XML format and is hereby incorporated by reference in its entirety. Said XML copy, created on Dec. 15, 2022, is named 127940-0103.xml and is 74,804 bytes in size.

### TECHNICAL FIELD

The invention relates to Fas Apoptosis Inhibitory Molecule (FAIM or FAIM1) or fragments and/or mimetics thereof, compositions containing FAIM or fragments and/or mimetics thereof, and methods of treatment and systems comprising FAIM or fragments and/or mimetics thereof.

### BACKGROUND OF THE INVENTION

A number of neurodegenerative diseases are associated with accumulation of damaged, misfolded proteins that form pathological soluble and/or insoluble assemblies, aggregates, and/or deposits, including Alzheimer’s disease (AD) (associated with accumulation of Amyloid beta (Aβ) peptide and/or Tau); Parkinson’s disease (PD) (associated with α-synuclein); Huntington’s disease (HD) (associated with Huntingtin with tandem glutamine repeats); amyotropic lateral sclerosis (ALS) (associated with Superoxide dismutase 1 and other aggregation-prone proteins); Multiple tauopathies (associated with Tau protein); Spongiform encephalopathies (associated with prion proteins); Familial amyloidotic polyneuropathy (associated with transthyretin); and chronic traumatic encephalopathy. In addition, other diseases may be associated with accumulation of damaged, misfolded proteins that form pathological soluble and/or insoluble assemblies, aggregates and/or deposits including diabetes, hemoglobinopathies, liver disease, and others.

At present, researchers hypothesize that molecules capable of binding and disassembling such protein assemblies, aggregates, and/or deposits may reverse disease progression and improve the lives of afflicted patients. Hsp104 is an ATP-binding protein found in yeast that dissolves stress-induced protein aggregates, but Hsp 104 has no metazoan homolog. A continuing search for Hsp 104-like activity among mammalian proteins has yielded few candidates.

### SUMMARY OF THE INVENTION

The present technology is directed to fragments of Fas Apoptosis Inhibitory Molecule (FAIM) or mimetics thereof, compositions containing FAIM or fragments and/or mimetics thereof, and methods of treatment and systems comprising FAIM or fragments and/or mimetics thereof.

In one aspect, the present technology is directed to fragment of FAIM or mimetics thereof. The technology provides a peptide or mimetic thereof comprising an amino acid sequence having at least 70% sequence identity to

MEDRSKTTNTWVLHMDGENFRIVLEKDTMDVWCNGKKLETAGEFVDDGTE
THFSIGNHDCYIKAVSSGKRKEGIIHTLIVDNREIPEIAS (SEQ ID N
O: 6).

In another aspect, the present technology provides a peptide or mimetic thereof comprising an amino acid sequence having at least 70% sequence identity to

MEDRSKTTNTW (SEQ ID NO: 7),

VLHMDGENFR (SEQ ID NO: 8),

IVLEKDTMDV (SEQ ID NO: 9),

WCNGKKLETA (SEQ ID NO: 10),

GEFVDDGTET (SEQ ID NO: 11),

HFSIGNHDCY (SEQ ID NO: 12),

IKAVSSGKRK (SEQ ID NO: 13),

EGIIHTLIVD (SEQID NO: 14),

or

NREIPEIAS (SEQ ID NO: 15).

The technology also provides a compositions including any of the FAIM peptides or fragments and/or mimetics thereof. In any embodiment, the peptide or mimetic thereof may include an amino acid sequence having at least 70% sequence identity to SEQ ID NO: 1, 2, 3, 4, 6, 7, 8, 9, 10, 11, 12, 13, 14, or 15, wherein the peptide has a length of at least 10 amino acid residues. In any embodiment, the peptide or mimetic thereof may include an amino acid sequence having at least 70% sequence identity to SEQ ID NO: 1, 2, or 3. In any embodiment, the composition may additionally include an agent that induces expression of the peptide and/or a clearing agent.

In another aspect, the present technology provides a method for treating a neurodegenerative or other proteinopathy in a subject in need thereof. The method may include administering a therapeutically effective amount of the composition to the subject in need thereof. In any embodiment, the neurodegenerative or other proteinopathy may include Alzheimer’s disease, Parkinson’s disease, Huntington’s disease, amyotropic lateral sclerosis, multiple tauopathies, spongiform encephalopathies, familial amyloidotic polyneuropathy, chronic traumatic encephalopathy, or a combination of two or more thereof.

Also provided is a method for treating a neurodegenerative or other proteinopathy in a subject in need thereof, the method comprising, administering a therapeutically effective amount of FAIM, a polynucleotide operable to encode and/or express FAIM, or an agonist of FAIM to the subject in need thereof.

### BRIEF DESCRIPTION OF THE DRAWINGS

Some embodiments will now be described, by way of example only and without waiver or disclaimer of other embodiments, with reference to the accompanying drawings, in which:

FIGS. 1a and 1b depict the results of FAIM KO germ cells under various conditions. Here, cells are susceptible to heat/oxidative stress-induced cell death. GC-2spd(ts) cells, were incubated under stress conditions as noted, for the indicated periods of time. Cell were also exposed to anti-FAS antibody for 24 hours. Cells were stained with 7-AAD and cell viability was analyzed by flow cytometer (a, b). Representative flow data are shown in a. A summary of pooled data from 3 independent experiments is shown in b. Data represent mean ± SEM. HS, heat shock; MN, menadione.

FIGS. 2a-2b - FAIM knockout clones GC-2spd(ts) and HeLa cells lack expression of FAIM protein. Western blot analyses of FAIM protein expression levels using cell lysates are shown for GC-2spd(ts) (a) and HeLa cells (b).

FIGS. 3a-3f - FAIM KO cells are susceptible to heat/oxidative stress-induced cell death. HeLa cells (a-d) or mouse primary fibroblasts (e, f), were incubated under stress conditions as noted for the indicated periods of time. a,b, WT HeLa cells and FAIM KO HeLa cells were stained with 7-AAD and cell viability was analyzed by flow cytometry after exposure to heat shock and menadione (MN)-induced oxidative stress. Representative flow data (a) and a summary of pooled data from 3 independent experiments (b) are shown. c,d, Cell viability was determined by supernatant LDH leaked from WT HeLa cells and FAIM KO HeLa cells upon heat shock (c) or upon menadione-induced oxidative stress (d), as indicated. Pooled data from 3 independent experiments are shown. e,f, Primary fibroblasts from WT and FAIM KO mice were subjected to menadione-induced (e) and arsenite-induced (f) oxidative stress in vitro, and cell viability was determined by supernatant LDH. Pooled data from 3 independent experiments are shown.

FIGS. 4a-4b - FAIM KO mice lack exons 3-5. a, Schematic representation of the targeting vector and the targeted allele of the mouse FAIM gene. b, Genotype determination of FAIM mice by PCR. Multiplex PCR genotyping analyses for KO (389 bp) and WT (514 bp) FAIM genes were performed to confirm the genotypes of wild-type (^{+/+}), heterozygous (^{+/-}) and homozygous (^{-/-}) mice. Representative genotyping results are shown.

FIGS. 5a-5e - Caspase-dependent apoptosis and ROS production are normal in FAIM KO HeLa cells under stress conditions. a, ROS production was measured by CellRox deep red staining reagent after oxidative stress induction. b, Apoptosis induction was assessed by monitoring Caspase3/7 activation with CellEvent caspase3/7 detection reagent after oxidative stress induction as indicated. c-e, Cell disruption was determined by LDH release with or without the pan-caspase inhibitor, Z-VAD-fmk, under oxidative stress conditions as indicated. Caspase-dependent cell death (d) and caspase-independent cell death (e) were calculated based on c. A summary of pooled data from 3 independent experiments is shown. Data represent mean ± SEM. MN, menadione.

FIG. 6 - FAIM mRNA expression does not change during cellular stress induction. FAIM mRNA expression levels during heat shock conditions were analyzed by qPCR. Primers for HSPβ5 (αB-crystallin), HSP70 A1A or HSP90 AA1 were also used as positive controls of heat stress induced genes. R2; recovery at 37° C. for 2 hours after heat stress at 43° C. for 2 hours. R6; recovery at 37° C. for 6 hours after heat stress at 43° C. for 2 hours. A summary of pooled data from 2 independent experiments is shown. Data represent mean ± SEM.

FIGS. 7a-7b - FAIM protein shifts to the detergent-insoluble fraction after stress induction in HeLa cells. a, HeLa cells were exposed to heat shock (HS) at 43° C. for 1 hour or for 2 hours, and were incubated at 37° C. for 6 or 18 hours recovery (R6 and R18) after incubation at 43° C. for 2 hours. HeLa cells were subjected to oxidative stress by treatment with 100 µM menadione (MN) for the indicated times (vehicle control; DMSO for 18 hours). After stress induction, cells were harvested, soluble proteins were isolated using RIPA buffer and RIPA buffer-insoluble proteins were extracted. Equal amounts of protein for each fraction were analyzed by western blotting for FAIM, HSP27, and actin as a loading control. b, HeLa cells were incubated at 37° C., or were exposed to heat shock (43° C.) for 2 hours. Cells were then harvested and proteins were divided into 4 fractions, 1; cytosol (MEK½-containing), 2; membrane/organella (AIF-containing), 3; nucleus (histone H3-containing) and 4; cytoskeleton/insoluble (vimentin-containing). Equal amounts of protein were analyzed by western blotting. Representative data are shown for a and b. Similar results were obtained from 3 independent experiments.

FIG. 8 - The majority of FAIM protein shifts to the detergent-insoluble fraction with sHSPs after heat stress induction in HLE B-3 cells. After heat stress induction of HLE B-2 cells for 2 hours, cells were harvested and proteins were divided into the 4 fractions: cytosol (MEK½-containing), membrane/organella (AIF-containing), nucleus (histone H3-containing) and cytoskeleton/insoluble (vimentin-containing). Equal amounts of protein were analyzed by western blotting. Representative data are shown. Similar results were obtained from 2 independent experiments.

FIGS. 9a-9b - FAIM binds ubiquitinated proteins after cellular stress induction. FAIM-ubiquitin binding was assessed by co-immunoprecipitation (a) and in situ PLA (b). a, FAIM KO HeLa cells were transiently transfected with FLAG-tagged FAIM protein. Transfected FAIM KO HeLa cells were subjected to oxidative stress by incubation with menadione (MN, 100 µM) for 1 hour, or were incubated with DMSO (the diluent for menadione), after which cells were harvested. Lysates were immunoprecipitated with anti-FLAG and subjected to SDS-PAGE and western blotted for ubiquitin. b, FAIM KO HeLa cells and WT HeLa cells were subjected to heat shock (HS) at 43° C. or oxidative stress (MN, 100 µM), as indicated, and then fixed and permeabilized, after which PLA reaction was carried out to detect proximity of FAIM and ubiquitin. Red dots indicate PLA positive signals and nuclei are stained blue with DAPI. Similar results were obtained from at least 2 independent experiments.

FIGS. 10a-10f - FAIM-deficient cells accumulate ubiquitinated, aggregated proteins in the detergent-insoluble fraction after stress induction. a, WT HeLa cells and FAIM KO HeLa cells were incubated at 37° C. or subjected to heat shock at 43° C. for 2 hours followed by recovery at 37° C. for 4 hours (R4) and for 18 hours (R18). Cells were lysed and detergent soluble and detergent insoluble fractions were isolated. Equal amounts of protein for each fraction were analyzed by western blotting for ubiquitin and actin as a loading control. b, WT HeLa cells and FAIM KO HeLa cells were incubated with menadione (MN) at 100 µM for the times indicated, or were incubated with DMSO vehicle. Cells were then handled as in a. c, WT HeLa cells and FAIM KO HeLa cells were incubated with menadione (MN) at 100 µM for the indicated times, after which aggregated proteins were filter trapped and blotted with anti-ubiquitin. d, Spleen and liver tissue from FAIM KO mice and their littermate controls were collected 18 hours after intraperitoneal administration of PBS or menadione (MN, 200 mg/kg). Tissue lysates were immediately extracted and protein samples were subjected to SDS-PAGE and western blotted for ubiquitin and actin as a loading control. Results shown in a-d are representative of at least 3 independent experiments. e,f, Serum samples obtained from mice treated as in d were analyzed for content of LDH (e) and ALT (f). Data represent mean ± SEM (n=7).

FIGS. 11a-11b - FAIM-deficient primary fibroblasts accumulate ubiquitinated, aggregated proteins in the detergent-insoluble fraction after stress induction. a, Primary mouse skin fibroblasts from WT and FAIM KO mice were incubated with menadione (MN) at 40 µM for the times indicated, or were incubated with DMSO vehicle. Cells were lysed and detergent soluble and detergent insoluble fractions were isolated. Equal amounts of protein for each fraction were analyzed by western blotting for Ubiquitin, and actin as a loading control. b, Primary mouse skin fibroblasts from WT and FAIM KO mice were incubated with menadione at 40 µM for the times indicated, or were incubated with DMSO vehicle. Aggregated proteins were filter trapped and blotted with anti-ubiquitin. Representative data are shown (a, b). Similar results were obtained from 2 independent experiments (a, b).

FIGS. 12a-12h - FAIM KO cells accumulate aggregation-prone proteins. a-d, WT HeLa cells and FAIM KO HeLa cells were transiently transfected with expression vectors for huntingtin (Htt)-Q23 and mutant Htt-Q74 that incorporate an eGFP tag. a, b, Two days later, eGFP⁺ cells (a) were evaluated for pulse-width vs pulse-height (b). The gated area represents cells expressing aggregated proteins. Results representative of 3 independent experiments are shown. c, WT and FAIM KO HeLa cells were transfected as in a and harvested at the indicated times. Percentages of cells expressing aggregated proteins out of total eGFP⁺ cells are shown. Data represent mean ± SEM from 3 independent experiments. d, WT and FAIM KO HeLa cells were transfected as in a and harvested 2 days later. Equal amounts of total cell lysates were subjected to FTA and stained with anti-GFP. Similar results were obtained in at least 3 independent experiments. e-h, WT HeLa cells and FAIM KO HeLa cells were transiently transfected with expression vectors for WT and G93A mutant SOD1. Cells were analyzed as in a-d.

FIGS. 13a-13c - Recombinant FAIM-S and FAIM-L suppress fibrillization/aggregation in a cell-free system. a, Spontaneous aggregation of β-amyloid (15 µM) in vitro was monitored by ThT assay over a period of 5 hours in the presence of recombinant FAIM-S, FAIM-L, HSP27, αB-crystallin or BSA at the doses indicated (blue, ThT alone; orange, 0.5 µM; gray 1 µM; yellow, 2 µM). ThT fluorescence was recorded every 5 minutes. b, Samples at 5 hours were subjected to SDS-PAGE and western blotted for amyloid. c, Aggregation of α-synuclein A53T mutant protein (20 µM) induced by 50 mM DTT (no DTT, left hand panel; 50 mM DTT, right hand panel) was monitored by ThT assay over a period of 48 hours in the presence of recombinant human FAIM-S and FAIM-L. ThT fluorescence was recorded every 20 minutes (blue; PBS, orange; FAIM-S, gray; FAIM-L). Representative data from at least 3 independent experiments are shown.

FIGS. 14a-14f - Recombinant FAIM-S and FAIM-L reverse β-amyloid, α-synuclein, and SOD1 aggregates. (a-c) Pre-aggregated β-amyloid (a), α-synuclein A53T (b) or SOD1 G93A (c) was incubated with or without 8 µM recombinant proteins for 2.5 hr. Aggregation status was monitored by ThT fluorescence. Data are shown as reduction of percent ThT fluorescence compared to that of negative controls and are expressed as mean ± SEM from 3 independent experiments. (d-f) Pre-aggregated β-amyloid (d), α-synuclein A53T (e) or SOD1 G93A (f) was incubated with or without 8 µM recombinant proteins for 2.5 hr, followed by centrifugation and SDS-PAGE of supernatant and pellet fractions. Results are representative of 3 independent experiments.

FIGS. 15a- 15f - Recombinant FAIM-S and FAIM-L reverse protein aggregates. Pre-aggregated β-amyloid (a, b), α-synuclein A53T (c, d) or SOD1 G93A (e, f) was incubated with or without 8 µM recombinant proteins for 2.5 hr. Aggregation status was monitored by FTA. a, c, e, Representative data from 3 independent experiments are shown. b, d, f, Densitometry quantification of FTA data from 3 independent experiments is shown.

FIG. 16 - Alignment of selected FAIM protein sequences from publicly available databases. Protein sequences of FAIM among the indicated species were aligned using the Clustal Omega program. Asterisks (*) denote single, fully conserved residues. Colons (:) denote conservation of strong groups, and periods (.) denote conservation of weak groups. No symbol indicates no consensus. Fig. discloses SEQ ID NOS 67-78, respectively, in order of appearance.

FIG. 17. Alignment of FAIM sequences in human, mouse, and C. elegans. Hu= human, mo. = mouse and ce. = C. elegans. Fig. discloses SEQ ID NOS 67, 70, and 73, respectively, in order of appearance.

FIG. 18 depicts results of recombinant FAIM prevents mutant SOD1-G93A aggregation in a cell-free system. Spontaneous aggregation of WT SOD1 and mutant SOD1-G93A (10 µM) in vitro was monitored by ThT assay in the presence of the reducing agent TCEP (tris(2-carboxyethyl)phosphine) (Sigma-Aldrich®) at 20 mM and EDTA at 5 mM, plus an extreme-temperature slippery PTFE Teflon® beads (McMaster-Carr), over a period of 48 hours in the presence or absence of recombinant FAIM (4 µM). ThT fluorescence was recorded every 10 minutes. Representative data from at least 3 experiments are shown.

FIGS. 19a, 19b, and 19c depict results from recombinant FAIM disassembles mutant SOD1-G93A aggregates in a cell-free system. Pre-aggregated SOD1 G93A, produced as described in Methods (in the section on generation of pre-formed protein aggregates), was incubated with or without 8 µM recombinant FAIM protein for 2.5 hr. (FIG. 19A) Aggregation status was monitored by ThT fluorescence as described in the legend to FIG. 18. Aggregation status was measured by FTA, as described in the legend to FIGS. 12 with membranes blotted with anti-SOD1 antibody. Aggregation measured by filter trap density was about half that of the buffer (control) (FIG. 19b). Densitometry quantification of FTA data are shown as reduction as compared to that of negative controls and are expressed as mean ± SEM from 3 independent experiments. (FIG. 19C) Pre-aggregated mutant SOD1-G93A was incubated with or without recombinant FAIM protein at the micromolar doses indicated for 2.5 hr, followed by centrifugation and separation of supernatant (S) and pellet (P) fractions that were subjected to SDS-PAGE under reducing conditions and western blotted with anti-SOD1 antibody (arrow). The locations of molecular weight markers in kDa are shown. “Pre” indicates SOD1-G93A in assembly buffer, before addition of FAIM. “Buffer” indicates Pre SOD1-G93A after addition of diluent buffer for FAIM (PBS). Digitally added vertical yellow lines were added to separate pairs of lanes representing supernatant and pellet fractions. Results shown are representative of 3 independent experiments. A Values of p<0.05 are considered statistically significant (*p<0.05, **p<0.01 or ***p<0.001).

FIG. 20 depicts an amino acid sequence alignment among different species. Here, Protein sequences of FAIM among the indicated species were aligned using the Clustal Omega program. Asterisks (*) denote single, fully conserved residues. Colons (:) denote conservation of strong groups, and periods (.) denote conservation of weak groups. No symbol indicates no consensus. Fig. discloses SEQ ID NOS 67-78, respectively, in order of appearance.

FIG. 21 depicts rapidly increased phosphorylated tau levels in the frontal cortex and hypothalamus regions of the FAIM KO (FAIM-deficient) mice as compared to wild-type (normal) mice (12 month of age) by immunohistochemistry using clone AT8 (mouse anti-phospho tau monoclonal antibody).

FIG. 22 depicts results showing recombinant FAIM c-terminal half (90-179) prevents aggregation/fibrillization of β-amyloid in a cell-free system with activity comparable to native full length FAIM. Spontaneous aggregation of β-amyloid (5 µM) in vitro was monitored by ThT assay over a period of 2 hours in the presence of 2 µM recombinant FAIM-S, FAIM-S c-terminal, or β-lactoglobulin B (BLGB). ThT fluorescence was recorded every 5 minutes.

FIG. 23 depicts results showing FAIM-deficient dopaminergic neurons accumulate intracellular α-syn aggregates in the detergent-soluble fraction (protofibrils) and in the detergent-insoluble fraction (mature fibrils) as judged by western blot (WB), unlike FAIM-sufficient neurons. FAIM-deficient dopaminergic neurons and isogenic controls (FAIM-sufficient) were derived from healthy donor’s induced pluripotent stem cells (iPSCs) and were transfected with pCMV-PCNA plasmid using NeuroMag transfection reagent (OZ Biosciences®) and then treated with or without seed-α-syn. After incubation for 7 days, cells were harvested and lysed. Sarkosyl-soluble (supernatant) and Sarkosyl-insoluble (pellet) fractions were isolated. Equal amounts of protein for each fraction were analyzed by western blotting (4-15% gradient PAGE gel). Membranes were probed using anti-phospho-Ser129-α-syn, anti-β-actin (loading control), and anti-FAIM antibodies.

### DETAILED DESCRIPTION OF THE INVENTION

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1 - 10, or 2 - 9, or 3 - 8, it is also envisioned that Parameter X may have other ranges of values including 1 - 9, 1 - 8, 1 - 3, 1 - 2, 2 - 10, 2 - 8, 2 - 3, 3 - 10, and 3 - 9.

As is understood by one skilled in the art, reference to “about” a value or parameter herein includes (and describes) embodiments that are directed to that value or parameter per se or that have a variance plus or minus of that value ranging from less than 10%, or less than 9%, or less than 8%, or less 7%, or less than 6%, or less than 5%, or less than 4%, or less than 3%, or less than 2%, or less than 1%, or less than 0.1 % than the stated value . For example, description referring to “about X” includes description of “X”.

FAIM molecules are a recently discovered family of evolutionarily conserved proteins structurally unrelated to other DR-induced apoptosis inhibitors. Human FAIM1 is located in the long arm of chromosome 3 (3q22.3), and it contains six exons and three putative translational start sites in exon 3. FAIM was originally cloned as a FAS antagonist in mouse primary B lymphocytes. A subsequent study identified the alternatively spliced form, termed FAIM-Long (L), which has 22 additional amino acids at the N-terminus. Thus, the originally identified FAIM was renamed FAIM-Short (S) (isolated from Fas-resistant B lymphocytes and described as an approximately 20 kDa soluble protein that is ubiquitously expressed and capable of inhibiting Fas-induced cell death). FAIM-L is expressed almost exclusively in the brain and in the testis whereas FAIM-S is ubiquitously expressed. See Mol Immunol. 2001;38: 65-72, the disclosure of which is incorporated herein by reference in its entirety. FAIM was disclosed in U.S. Pat. No. 6,683,168, which is herein incorporated by reference. Recently, the FAIM-Gm6432 gene, thought to be duplicated from the original FAIM gene, was identified in Muroidea rodents and its expression is limited to the testis.

Human FAIM1 is located in the long arm of chromosome 3 (3q22.3), and it contains six exons and three putative translational start sites in exon 3. See Schneider et al., A novel gene coding for a Fas apoptosis inhibitory molecule (FAIM) isolated from inducibly Fas-resistant B lymphocytes. J EXP MED. 1999; 189: 949-56, the disclosure of which is incorporated herein by reference in its entirety. With 66 more nucleotides than FAIM-S, FAIM-L is generated by the inclusion of exon 2b and is expressed mainly in neurons; however, FAIM-L has also been shown to be expressed in testes, and in the developing embryo. See Zhong et al., An alternatively spliced long form of Fas apoptosis inhibitory molecule (FAIM) with tissue-specific expression in the brain. MOL IMM 38 (2001) 65-72, the disclosure of which is incorporated herein by reference in its entirety. FAIM-L has a cytosolic distribution and exerts protection against TNFα- and Fas-induced apoptosis, thereby preventing the activation of caspase 8, and/or by interacting with and stabilizing the anti-apoptotic protein XIAP. FAIM-L also acts as a regulator in two neuronal processes that require caspase-3 activation, namely: axon-selective pruning and long-term depression. By stabilizing of XIAP levels and consequent caspase-3 inhibition, FAIM-L prevents these two processes in models of neuronal cells in vitro.

Intriguingly, in silico analysis indicates the existence of FAIM genes in the premetazoan genomes of single-celled choanoflagellates like M.brevicollis and S.rosetta, which is one of the closest living relatives of animals and a progenitor of metazoan life that first evolved over 600 million years age. S.rosetta contains only 9411 genes, out of which 2 faim genes were found. This evidence suggests that the FAIM gene evolved much earlier than many other genes and domains found in multicellular organisms, such as the death domain involved in animal cell apoptosis and implies that this gene may have another major function beyond apoptosis regulation. However, a lack of known effector/binding motifs and even partial sequence homology of FAIM with any other protein has to date rendered it difficult to predict such functions.

A series of overexpression studies demonstrated that FAIM produces resistance to FAS (CD95)-mediated apoptosis in B lymphocytes, HEK293T cells and PC12 cells, enhances CD40-mediated NF-κB activation in B lymphocytes, and induces neurite outgrowth in the PC12 cell line. Thus, FAIM expresses multiple activities related to cell death, signaling, and neural cell function. Nonetheless, the overarching physiological role of FAIM still remained unclear due to a lack of obvious phenotypic abnormalities of FAIM-deficient mice and cells.

The expression and evolution patterns of the faim and faim-gm6432 genes suggested that FAIM may be important for testicular functions. Testicular cells are highly susceptible to heat shock and oxidative stress, which in turn suggested that FAIM might be involved in the cellular stress response. We therefore hypothesized that FAIM might regulate cellular stress response pathways, including the disposition of misfolded and aggregated proteins, in testicular cells or even in other cell types.

In one aspect, the present technology is directed to fragment of FAIM or mimetics thereof. The technology provides a peptide or mimetic thereof comprising an amino acid sequence having at least 70% sequence identity to

MEDRSKTTNTWVLHMDGENFRIVLEKDTMDVWCNGKKLETAGEFVDDGTE
THFSIGNHDCYIKAVSSGKRKEGIIHTLIVDNREIPEIAS (SEQ ID N
O: 6)

. In any embodiment, the amino acid sequence may have at least 80%, 85%, 90%, 95%, or 99% sequence identity to SEQ ID NO: 6. In any embodiment, the peptide may exhibit the ability to disaggregate protein complexes. In any embodiment, the peptide may exhibit the ability to disaggregate protein complexes in the brain.

In another aspect, the present technology provides a peptide or mimetic thereof comprising an amino acid sequence having at least 70% sequence identity to

MEDRSKTTNTW (SEQ ID NO: 7)

,

VLHMDGENFR (SEQ ID NO: 8)

,

IVLEKDTMDV (SEQ ID NO: 9)

,

WCNGKKLETA (SEQ ID NO: 10)

,

GEFVDDGTET (SEQ ID NO: 11)

,

HFSIGNHDCY (SEQ ID NO: 12)

,

IKAVSSGKRK (SEQ ID NO: 13)

,

EGIIHTLIVD (SEQ ID NO: 14)

, or

NREIPEIAS (SEQ ID NO: 15)

. In any embodiment, the amino acid sequence may have at least 80%, 85%, 90%, 95%, or 99% sequence identity to SEQ ID NO: 7, 8, 9, 10, 11, 12, 13, 14, or 15. In any embodiment, the peptide may have a length of at least 10 amino acid residues. In any embodiment, the peptide may have a length of at least 15, at least 20, at least 25, at least 30, at least 40, or at least 50 amino acid residues. In any embodiment, the peptide may exhibit the ability to disaggregate protein complexes. In any embodiment, the peptide may exhibit the ability to disaggregate protein complexes in the brain.

In another aspect, the present technology provides a method for treating a neurodegenerative or other proteinopathy in a subject in need thereof, the method comprising, administering a therapeutically effective amount of the composition disclosed herein to the subject in need thereof. In any embodiment, the composition may include the peptide or mimetic thereof including an amino acid sequence having at least 70% sequence identity to SEQ ID NO: 1, 2, 3, 4, 6, 7, 8, 9, 10, 11, 12, 13, 14, or 15, wherein the peptide has a length of at least 10 amino acid residues. In any embodiment, the peptide or mimetic thereof may include an amino acid sequence having at least 70% sequence identity to SEQ ID NO: 1, 2, or 3. In any embodiment, the peptide or mimetic thereof may include an amino acid sequence having at least 80%, 90%, 95%, or 99% sequence identity to SEQ ID NO: 1, 2, or 3.

Also provided is a method for treating a neurodegenerative or other proteinopathy in a subject in need thereof, the method comprising, administering a therapeutically effective amount of FAIM, a polynucleotide operable to encode and/or express FAIM, or an agonist of FAIM to the subject in need thereof.

In any embodiment, neurodegenerative or other proteinopathy may include a neurodegenerative disease or condition in which at least one physiological event that contributes, or is associated with the neurodegenerative proteinopathy is the presence of misfolded proteins in the brain, neurons (e.g., neurons of the central or peripheral nervous system), and/or spinal column, of the subject with the neurodegenerative disease or condition. Examples of neurodegenerative proteinopathies that can be treated with the compositions of the present disclosure include, but are not limited to, Alzheimer’s disease (AD), Parkinson’s disease (PD), Huntington’s disease (HD), amyotropic lateral sclerosis (ALS), Multiple tauopathies, Spongiform encephalopathies, Familial amyloidotic polyneuropathy and, chronic traumatic encephalopathy.

The complete amino acid sequence of an exemplary human FAIM has the amino acid sequence:

“MTDLVAVWDVALSDGVHKIEFEHGTTSGKRVVYVDGKEEIRKEWMFKLV
GKETFYVGAAKTKATISIDAISGFAYEYTLEINGKSLKKYMEDRSKTTNT
WVLHMDGENFRIVLEKDTMDVWCNGKKLETAGEFVDDGTETHFSIGNHDC
YIKAVSSGKRKEGIIHTLIVDNREIPEIAS”

(NCBI Accession No.: CAG33403 (179 amino acids) as set for in SEQ ID NO: 1). Two different isoforms of human FAIM include isoforms “a” and “b”;

FAIM isoform “b” having amino acid sequence

“MASGDDSPIFEDDESPPYSLEKMTDLVAVWDVALSDGVHKIEFEHGTTS
GKRVVYVDGKEEIRKEWMFKLVGKETFYVGAAKTKATINIDAISGFAYEY
TLEINGKSLKKYMEDRSKTTNTWVLHMDGENFRIVLEKDAMDVWCNGKKL
ETAGEFVDDGTETHFSIGNHDCYIKAVSSGKRKEGIIHTLIVDNREIPEI
AS” (SEQ ID NO: 2

(NCBI▫Accession▫No.: NP_001028203; 201 amino acids - FAIM1 isoform b).

FAIM1 isoform “a” having the amino acid sequence

“MLLPFIRTLPLLCYNHLLVSPDSATLSPPYSLEKMTDLVAVWDVALSDG
VHKIEFEHGTTSGKRVVYVDGKEEIRKEWMFKLVGKETFYVGAAKTKATI
NIDAISGFAYEYTLEINGKSLKKYMEDRSKTTNTWVLHMDGENFRIVLEK
DAMDVWCNGKKLETAGEFVDDGTETHFSIGNHDCYIKAVSSGKRKEGIIH
TLIVDNREIPEIAS” (SEQ ID NO: 3

(NCBI Accession No. NP_001028202; 213 amino acids - FAIM1 isoform a)). The complete amino acid sequence of an exemplary murine FAIM has a NCBI Accession No.: AAD23879, version AAD23879.1 (residues 1-179) (having the amino acid sequence:

MTDLVAVWDV
ALSDGVHKIE FEHGTTSGKR VVYVDGKEEI RREWMFKLVG KETFFVGAAK
TKATINIDAI SGFAYEYTLE IDGKSLKKYM ENRSKTTSTW VLRLDGEDLR
VVLEKDTMDV WCNGQKMETA GEFVDDGTET HFSVGNHGCY IKAVSSGKRK
EGIIHTLIVD NREIPELTQ (SEQ ID NO: 4)

. See Schneider et al., A novel gene coding for a Fas apoptosis inhibitory molecule (FAIM) isolated from inducibly Fas-resistant B lymphocytes. J. Exp. Med. 189 (6), 949-956 (1999), the disclosure of which in incorporated by reference herein in its entirety.

By isolated and “substantially pure” is meant a protein or polypeptide that has been separated and purified to at least some degree from the components that naturally accompany it. Typically, a polypeptide is substantially pure when it is at least about 60%, or at least about 70%, at least about 80%, at least about 90%, at least about 95%, or even at least about 99%, by weight, free from the proteins and naturally-occurring organic molecules with which it is naturally associated. For example, a substantially pure protein or polypeptide may be obtained by extraction from a natural source, by expression of a recombinant nucleic acid in a cell that does not normally express that protein, or by chemical synthesis. An “isolated” FAIM protein is one which has been separated from a component of its natural environment. In some embodiments, FAIM is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) analysis.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a FAIM” refers to one or more nucleic acid molecules encoding a FAIM protein (or protein disaggregation functional fragment thereof), including such nucleic acid molecule(s) in a single vector or separate vectors, and such nucleic acid molecule(s) present at one or more locations in a host cell.

The term “recombinant” as used herein to describe a nucleic acid molecule, means a polynucleotide of genomic, mRNA, cDNA, viral, semisynthetic, and/or synthetic origin, which, by virtue of its origin or manipulation, is not associated with all or a fragment of the polynucleotide with which it is associated in nature, thus it non-natural. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

By “wild type” or “WT” or “native” herein is meant an amino acid sequence or a nucleotide sequence that is found in nature, including allelic variations.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “detection” includes any means of detecting, including direct and indirect detection.

The term “biomarker” as used herein refers to an indicator, e.g., a predictive, diagnostic, and/or prognostic indicator, which can be detected in a sample. The biomarker may serve as an indicator of a particular subtype of a disease or disorder (e.g., cancer) characterized by certain, molecular, pathological, histological, and/or clinical features. In some embodiments, the biomarker is a gene. In some embodiments, the biomarker is a variation (e.g., mutation and/or polymorphism) of a gene. In some embodiments, the biomarker is a translocation. Biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers.

The “presence,” “amount,” or “level” of a biomarker associated with an increased clinical benefit to an individual is a detectable level in a sample. These can be measured by methods known to one skilled in the art and also disclosed herein. The expression level or amount of biomarker assessed can be used to determine the response to the treatment.

The term “diagnosis” is used herein to refer to the identification or classification of a molecular or pathological state, disease or condition (e.g., an inflammatory disease, for example, inflammatory bowel disease). For example, “diagnosis” may refer to identification of a particular type of neurodegenerative proteinopathy disease, for example, Alzheimer’s disease. “Diagnosis” may also refer to the classification of a particular subtype of disease, e.g., by histopathological criteria, or by molecular features (e.g., a subtype characterized by expression of one or a combination of biomarkers (e.g., particular genes or proteins encoded by said genes)).

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., protein disaggregation values). The difference between said two values may be, for example, less than about 20%, less than about 10%, and/or less than about 5% as a function of the reference/comparator value.

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., protein disaggregation values). The difference between said two values may be, for example, greater than about 10%, greater than about 20%, greater than about 30%, greater than about 40%, and/or greater than about 50% as a function of the value for the reference/comparator molecule.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, a carrier, a diluent, a stabilizer, or a preservative.

The terms “subject” and “individual” and “patient” are used interchangeably herein, and refer to an animal, for example a mammal, for example, a human or non-human mammal, to whom treatment, including prophylactic treatment, with a pharmaceutical composition as disclosed herein, is provided. The term “subject” as used herein refers to human and non-human animals. The term “non-human animals” includes all vertebrates, e.g., mammals, such as non-human primates, (particularly higher primates and monkeys), sheep, dogs, rodents (e.g. mouse or rat), guinea pigs, goats, pigs, cats, rabbits, cows, and non-mammals such as chickens, amphibians, reptiles etc. In one embodiment, the subject is human. In another embodiment, the subject is an experimental animal or animal substitute as a disease model. Non-human mammals include mammals such as non-human primates, (particularly higher primates and monkeys), sheep, dogs, rodents (e.g. mouse or rat), guinea pigs, goats, pigs, cats, rabbits and cows. In some aspects, the non-human animal is a companion animal such as a dog or a cat.

As used herein, “treatment” (and variations such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the disclosure (e.g., antibodies targeting one or more of the proteins discussed herein) are used to delay development of a disease or to slow the progression of a disease, or to prevent, delay or inhibit the development of a side effect related to the treatment of a different disease being actively treated.

By “reduce” or “inhibit” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., NF-κB activity) or binding). In some embodiments, reduce or inhibit can refer to the relative reduction of a side effect associated with a treatment for a condition or disease.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. MoI. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One illustrative example of an algorithm that is suitable for determining percent sequence identity and sequence similarity is the BLAST algorithm, which is described by Altschul et al. (J. MoI. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=-4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

As used herein a “peptide or polypeptide fragment” refers to a protein or polypeptide that is a fragment of a comparator protein or polypeptide. In any embodiment, the peptide or polypeptide fragment may be at least 5% of the total comparator protein or polypeptide including 10%, 15%, 20%, 25%, 30%, 35%, 40%, 45%, 50%, 55%, 60%, 65%, 70%, 75%, 80%, 85%, 90%, or 95% of the total comparator protein or polypeptide. The peptide or polypeptide fragment may include the N-terminus, the C-terminus, or parts between the N-terminus and the C-terminus of the comparator protein or polypeptide. The peptide or polypeptide fragment may include the C-terminus of the comparator protein or polypeptide. In any embodiment, the peptide or polypeptide fragment of the comparator protein or polypeptide may include at least 10 amino acids. In any embodiment, the peptide or polypeptide fragment of the comparator protein or polypeptide may include at least 15, 20, 25, 30, 35, 40, 45, or 50 amino acids. In any embodiment, the peptide or polypeptide fragment of the comparator protein or polypeptide may include 10 to 200 amino acids including 15-175, 20-150, 30-140, 40-130, 50-120, 60-115, 70-110, or 80-100 amino acids. In any embodiment, the peptide or polypeptide fragment may further be a variant or mimetic. In any embodiment, the peptide or polypeptide fragment of the comparator protein or polypeptide may be a peptide including an amino acid sequence having at least 70% identity to

MEDRSKTTNTWVLHMDGENFRIVLEKDTMDVWCNGKKLETAGEFVDDGTE
THFSIGNHDCYIKAVSSGKRKEGIIHTLIVDNREIPEIAS (SEQ ID N
o: 6)

. In any embodiment, the peptide or polypeptide fragment of the comparator protein or polypeptide may be a peptide including an amino acid sequence having at least 80% identity to SEQ ID No: 6. In any embodiment, the fragment of the comparator protein or polypeptide may be a peptide including an amino acid sequence having at least 90% identity to SEQ ID No: 6. In any embodiment, the peptide or polypeptide fragment of the comparator protein or polypeptide may be a peptide including an amino acid sequence having at least 95% identity to SEQ ID No: 6. In any embodiment, the peptide or polypeptide fragment of the comparator protein or polypeptide may be a peptide including an amino acid sequence having at least 99% identity to SEQ ID No: 6. In any embodiment, the comparator protein or polypeptide may be the protein of SEQ ID No: 1, 2, 3, or 4.

The term “variant” or “mimetic” (used interchangeably) as used herein refers to a protein, polypeptide or nucleic acid that differs from the comparator protein, polypeptide or nucleic acid by one or more amino acid or nucleic acid deletions, additions, substitutions or side-chain modifications, yet retains one or more specific functions or biological activities of the naturally occurring molecule, for example, the ability to disaggregate protein complexes in the brain of a subject treated with the compositions of the present disclosure. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. Substitutions encompassed by the present disclosure may also be “non-conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments amino acid substitutions are conservative. Also encompassed within the term variant or mimetic when used with reference to a polynucleotide, protein, or polypeptide, refers to a protein, polynucleotide or polypeptide that can vary in primary, secondary, or tertiary structure, as compared to a reference polynucleotide, protein, or polypeptide, respectively (e.g., as compared to a wild-type polynucleotide, protein, or polypeptide).

Variants or mimetics can also be synthetic, recombinant, or chemically modified polynucleotides, proteins, or polypeptides isolated or generated using methods well known in the art. Variants or mimetics can include conservative or non-conservative amino acid changes, as described below. Polynucleotide changes can result in amino acid substitutions, additions, deletions, fusions and truncations in the polypeptide encoded by the reference sequence. Variants or mimetics can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a protein or polypeptide, refers to a change in the amino acid composition of the protein or polypeptide that does not substantially alter the protein or polypeptide’s activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine.

In any embodiment, the proteins and polypeptides described herein may also be a mimetic protein or polypeptide.

“Conservative amino acid substitutions” as referenced herein result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for a protein or polypeptide’s activity or substitution of amino acids with other amino acids having similar properties (e.g., acidic, basic, positively or negatively charged, polar or non-polar, etc.) such that the substitution of even critical amino acids does not reduce the activity of the protein or polypeptide, (i.e. the ability of the protein or polypeptide to penetrate the blood brain barrier (BBB)). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can also select conservative amino acid substitutions encompassed suitable for amino acids on the interior of a protein or polypeptide, for example one can use suitable conservative substitutions for amino acids is on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants or mimetics.

The term “derivative” as used herein refers to peptides which have been chemically modified, for example but not limited to by techniques such as ubiquitination, labeling, pegylation (derivatization with polyethylene glycol), lipidation, glycosylation, or addition of other molecules. A molecule also a “derivative” of another molecule when it contains additional chemical moieties not normally a part of the molecule. Such moieties can improve the molecule’s solubility, absorption, biological half-life, etc. The moieties can alternatively decrease the toxicity of the molecule, eliminate or attenuate any undesirable side effect of the molecule, etc. Moieties capable of mediating such effects are disclosed in Remington’s Pharmaceutical Sciences, 18th edition, A. R. Gennaro, Ed., Mack Publ., Easton, Pa. (1990), incorporated herein, by reference, in its entirety.

The term “functional” when used in conjunction with “fragment” “mimetic”, “derivative” or “variant” refers to a protein or polypeptide of the disclosure which possesses a biological activity (either functional or structural) that is substantially similar to a biological activity of the entity or molecule it is a functional derivative or functional variant thereof, i.e., a protein or polypeptide that disaggregates protein complexes into either smaller complexes or soluble fragments of complexes, for example, wherein said protein complex disaggregation provides some therapeutic benefit and/or that the smaller complexes or soluble fragments of complexes do not cause or exacerbate the conditions, symptoms or pathology of the disease being treated.

The term “substitution” when referring to a peptide, refers to a change in an amino acid for a different entity, for example another amino acid or amino-acid moiety. Substitutions can be conservative or non-conservative substitutions.

A “mimetic” or “analog” of a molecule such as a FAIM protein refers to a molecule similar in function to either the entire FAIM molecule or to a fragment thereof. The term “analog” or “mimetic” is also intended to include allelic species and induced variants. Analogs and mimetics typically differ from naturally occurring proteins at one or a few amino acid positions, often by virtue of conservative substitutions, or may include deletion of the primary structure of the entire FAIM molecule, but which retains the protein disaggregation activity of the FAIM molecule. Analogs and mimetics typically exhibit at least 70%, or 80%, or 85% or 90% or 95% or 99% sequence identity with natural FAIM proteins. Some analogs and/or mimetics also include unnatural amino acids or modifications of N or C terminal amino acids. Examples of unnatural amino acids are, for example but not limited to; disubstituted amino acids, N-alkyl amino acids, lactic acid, 4-hydroxyproline, γ-carboxyglutamate, N,N,N-trimethyllysine, N-acetyllysine, phosphoserine, N-acetylserine, N-formylmethionine, 3-methylhistidine, 5-hydroxylysine, N-methylarginine. Mimetics and analogs can be screened for prophylactic or therapeutic efficacy in in vitro cellular models, animal models for example, transgenic animal models as described below.

The term “fusion protein” as used herein refers to a recombinant protein of two or more proteins or two or more peptides or to one or more peptides and one or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. Fusion proteins can include an epitope tag, marker tag, or a half-life extender such as polymers of polyethyleneglycol (PEG). Epitope tags include biotin, FLAG, c-myc, hemaglutinin, agglutinin, His6 (SEQ ID NO: 16), maltose binding protein (MBP), digoxigenin, marker tags can include FITC, Cy3, Cy5, green fluorescent protein (GFP), enhanced green fluorescent protein (EGFP), V5 epitope tags, GST, β-galactosidase, AU1, AU5, and avidin. Half-life extenders include Fc domain, acyl-lipophillic molecules, polyethylene glycol polymers of various lengths, and serum albumin. In some embodiments, a FAIM fusion protein comprises a FAIM protein operably linked to a TAT peptide. A “TAT” peptide is a cell penetrating peptide that is well known in the art, and is used for cell permeability; here, fusion with a TAT peptide would enable FAIM to penetrate any cell. In yet other embodiments, a fusion protein can be a FAIM protein operably linked to a neuronal cell ligand, said ligand being specific to neuronal-cell-specific receptors. For example, in some embodiments, a FAIM-neuronal ligand fusion protein can comprise a FAIM protein operably linked to the low density lipoprotein receptor (LDLR)-binding domain of apolipoprotein B (apoB).

In various embodiments, pharmaceutical and/or non-pharmaceutical compositions are provided for herein that include one or more FAIM proteins or a fragment and/or mimetic thereof. In any embodiment, the compositions can additionally include one or more pharmaceutically acceptable excipient.

An exemplary formulation method can be adapted from Remington’s Pharmaceutical Sciences (17th Ed., Mack Pub. Co. 1985); Remington: Essentials of Pharmaceutics (Pharmaceutical Press, 2012), the disclosure of which is incorporated herein by reference in its entirety. In some embodiments, without limitation, the methods described herein can utilize formulations containing one or more isolated FAIM proteins or fragments and/or mimetic thereof, that are contained within a pharmaceutically acceptable vehicle, carrier, adjuvants, additives and/or excipient that allows for storage and handling of the agents before and during administration. Moreover, in accordance with certain aspects of the present disclosure, the agents suitable for administration may be provided in a pharmaceutically acceptable vehicle, carrier, or excipient with or without an inert diluent. Further, in addition to the above-described components, the formulation may contain additional lubricants, emulsifiers, suspending-agents, preservatives, or the like. Accordingly, the pharmaceutically acceptable vehicle, carrier, adjuvants, additives and/or excipient must be acceptable in the sense of being compatible with the other ingredients of the formulation and not deleterious to the recipient thereof, i.e., are sterile compositions and contain pharmaceutically acceptable vehicle, carrier, adjuvants, additives that are approved by the US Food and Drug Administration (FDA) for administration to a human subject.

Formulations containing one or more isolated FAIM proteins or fragments and/or mimetic thereof may be prepared with one or more carriers, excipients, and diluents. Exemplary carriers, excipients and diluents can include one or more of sterile saline, phosphate buffers, Ringer’s solution, and/or other physiological solutions that are used in the preparation of cellular therapies for administration in humans.

In certain embodiments, formulations comprising one or more isolated FAIM proteins or fragments and/or mimetic, can contain further additives including, but not limited to, pH-adjusting additives, osmolarity adjusters, tonicity adjusters, anti-oxidants, reducing agents, and preservatives. Useful pH-adjusting agents include acids, such as hydrochloric acid, bases or buffers, such as sodium lactate, sodium acetate, sodium phosphate, sodium citrate, sodium borate, or sodium gluconate. Further, the compositions of the invention can contain microbial preservatives. Useful microbial preservatives include methylparaben, propylparaben, and benzyl alcohol. The microbial preservative is typically employed when the formulation is placed in a vial designed for multidose use. Other additives that are well known in the art include, e.g., detackifiers, anti-foaming agents, antioxidants (e.g., ascorbyl palmitate, butyl hydroxy anisole (BHA), butyl hydroxy toluene (BHT) and tocopherols, e.g., alpha.-tocopherol (vitamin E)), preservatives, chelating agents (e.g., EDTA and/or EGTA), viscomodulators, tonicifiers (e.g., a sugar such as sucrose, lactose, and/or mannitol), flavorants, colorants, odorants, opacifiers, suspending agents, binders, fillers, plasticizers, lubricants, and mixtures thereof. The amounts of such additives can be readily determined by one skilled in the art, according to the particular properties desired. Further, the formulation may comprise different types of carriers suitable for liquid, solid, or aerosol delivery.

In certain embodiments, a formulation can be made by suspending one or more isolated FAIM proteins or a mimetic thereof in a physiological buffer with physiological pH, for example, a sterile buffer solution such as phosphate buffer solution (PBS); sterile 0.85% NaCl solution in water; or 0.9% NaCl solution in Phosphate buffer having KCl. Physiological buffers (i.e., a 1x PBS buffer) can be prepared, for example, by mixing 8 g of NaCl; 0.2 g of KCl; 1.44 g of Na₂HPO₄; 0.24 g of KH₂PO₄; then, adjusting the pH to 7.4 with HCl; adjusting the volume to 1L with additional distilled H₂O; and sterilizing by autoclaving.

When necessary, proper fluidity of the compositions and formulations described herein can be maintained, for example, by the use of a coating such as lecithin, by the maintenance of the required size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for such compositions comprising one or more isolated FAIM proteins or fragments and/or mimetic thereof. Furthermore, various additives which enhance the stability, sterility, and/or isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like. Prolonged absorption of the injectable pharmaceutical form can be brought about by the use of agents delaying absorption, for example, aluminum monostearate and gelatin. According to some embodiments of the present disclosure, however, any vehicle, diluent, or additive used would have to be compatible with one or more isolated FAIM proteins or a mimetic thereof.

Sterile injectable solutions can be prepared by incorporating one or more isolated FAIM proteins or a mimetic thereof utilized in practicing some embodiments of the present disclosure in the required amount of the appropriate solvent with various other ingredients, as desired.

In some non-limiting embodiments, a formulation can be prepared by combining one or more isolated FAIM proteins or fragments and/or mimetic thereof produced recombinantly or isolated from natural sources, such as from human cells and/or sera. Formulations containing one or more isolated FAIM proteins or fragments and/or mimetic thereof may be prepared with one or more carriers, excipients, and diluents. Exemplary carriers, excipients and diluents can include one or more of sterile saline, phosphate buffers, Ringer’s solution, and/or other physiological solutions that are used in the preparation of cellular therapies for administration in humans. In some embodiments, one or more isolated FAIM proteins or fragments and/or mimetic thereof may be lyophilized and packaged into sterile containers to be reconstituted with an appropriate volume of buffer or other excipients for immediate administration, in 25 mg vials, 50 mg vials, 75 mg vials, and 100 mg vials.

In any embodiment, the composition may include one or more agents that can enhance FAIM activity. In any embodiment, the composition may include one or more agents that can induce expression of FAIM or fragements and/or mimetics thereof. In any embodiment, the agent may include a polynucleotide. In any embodiment, the polynucleotide may include mRNA and/or complementary cDNA. In any embodiment, the polynucleotide may include human FAIM-S mRNA

(tgggtccgtggcggcgggaggggtggcctcctgcgctggtcgccccagg
ggacctgagaggcgcgacaaacagtcggcgcgtttggtactcgcgcctgc
agagctttcaacctccgcgccggctgcgcctgtttctcggccaggggagc
aaggccacgcggcctacgcagccgagtcggaaccaaccggttgtttggtg
aaacctaccccagagcctcccgcggcccacagagcacagccctccttaca
gcctagaaaaaATGACAGATCTCGTAGCTGTTTGGGATGTTGCTTTAAGT
GACGGAGTCCACAAGATCGAATTTGAACATGGGACTACATCAGGCAAACG
AGTAGTATATGTAGATGGAAAGGAAGAGATAAGAAAAGAGTGGATGTTCA
AATTAGTGGGCAAAGAAACATTCTATGTTGGAGCTGCAAAGACAAAAGCG
ACCATAAATATAGACGCTATCAGTGGTTTTGCTTATGAATATACTCTGGA
AATTAATGGGAAAAGTCTCAAGAAGTATATGGAGGACAGATCAAAAACCA
CCAATACTTGGGTATTACACATGGATGGTGAGAACTTTAGAATTGTTTTG
GAAAAAGATGCTATGGACGTATGGTGCAATGGTAAAAAATTGGAGACAGC
GGGTGAGTTTGTAGATGATGGGACTGAAACTCACTTCAGTATCGGGAACC
ATGACTGTTACATAAAGGCTGTCAGTAGTGGGAAGCGGAAAGAAGGGATT
ATTCATACTCTCATTGTGGATAATAGAGAAATCCCAGAGATTGCAAGTTA
Atgaattttcatcttaagaagtaaagatcaggactttttaattactgtgg
taattaaatgtgttcagtatgtacttatcagtacatttagtctgcaatgt
tttaattttttaaaaagttacatgaaactaacattccaagggtcaggaaa
aaaaccaattatgtatagtcataaaaattacaatttatgatgcaaataat
gtaaaatgttttaaagacaaatggcaaataagatatggaccaaagtcact
aatgttttacaacagtaacctttactataataaatactttt (SEQ ID
NO: 17))

,▫human FAIM-L mRNA

(tgggtccgtggcggcgggaggggtggcctcctgcgctggtcgccccagg
ggacctgagaggcgcgacaaacagtcggcgcgtttggtactcgcgcctgc
agagctttcaacctccgcgccggctgcgcctgtttctcggccaggggagc
aaggccacgcggcctacgcagccgagtcggaaccaaccggttgtttggtg
aaacctaccccagagcctcccgcggcccacagagcacagactgtttttgc
caaccATGGCATCTGGAGATGACAGTCCTATCTTTGAAGATGATGAAAGC
CCTCCTTACAGCCTAGAAAAAATGACAGATCTCGTAGCTGTTTGGGATGT
TGCTTTAAGTGACGGAGTCCACAAGATCGAATTTGAACATGGGACTACAT
CAGGCAAACGAGTAGTATATGTAGATGGAAAGGAAGAGATAAGAAAAGAG
TGGATGTTCAAATTAGTGGGCAAAGAAACATTCTATGTTGGAGCTGCAAA
GACAAAAGCGACCATAAATATAGACGCTATCAGTGGTTTTGCTTATGAAT
ATACTCTGGAAATTAATGGGAAAAGTCTCAAGAAGTATATGGAGGACAGA
TCAAAAACCACCAATACTTGGGTATTACACATGGATGGTGAGAACTTTAG
AATTGTTTTGGAAAAAGATGCTATGGACGTATGGTGCAATGGTAAAAAAT
TGGAGACAGCGGGTGAGTTTGTAGATGATGGGACTGAAACTCACTTCAGT
ATCGGGAACCATGACTGTTACATAAAGGCTGTCAGTAGTGGGAAGCGGAA
AGAAGGGATTATTCATACTCTCATTGTGGATAATAGAGAAATCCCAGAGA
TTGCAAGTTAAtgaattttcatcttaagaagtaaagatcaggacttttta
attactgtggtaattaaatgtgttcagtatgtacttatcagtacatttag
tctgcaatgttttaattttttaaaaagttacatgaaactaacattccaag
ggtcaggaaaaaaaccaattatgtatagtcataaaaattacaatttatga
tgcaaataatgtaaaatgttttaaagacaaatggcaaataagatatggac
caaagtcactaatgttttacaacagtaacctttactataataaatacttt
t (SEQ ID NO: 18))

, or a combination thereof.

In any embodiment, the composition may include one or more clearing agents that can aid in clearance of disaggregated protein complexes. In any embodiment, the one or more clearing agents may include an antibody directed against the target aggregated protein, such as donanemab (Lilly), solanezumab (Lilly) and gantenerumab (Roche) which are antibodies directed against β-amyloid, or combinations of two or more thereof.

In yet other embodiments, a composition can comprise agents that inhibit the activity of FAIM.

In various embodiments, useful compositions comprising one or more isolated FAIM proteins or fragments and/or mimetic thereof, whether pharmaceutical or non-pharmaceutically acceptable can contain from about 0.01 mg/kg to about 100 mg/kg (wt/wt%) of the patient’s weight.

The present inventors have experimentally shown that FAIM fulfills the previously unknown role of protection against stress in various kinds of cell types. The inventors have discovered that FAIM counteracts stress-induced loss of cellular viability in vivo and in vitro. In this process, FAIM localizes to detergent insoluble material and binds ubiquitinated aggregated proteins. Importantly, FAIM protects against protein aggregation and solubilizes previously established protein aggregates. These findings strongly suggest a novel, FAIM-specific role in holozoan protein homeostasis that may be relevant to the pathophysiology of neurodegenerative diseases.

As used herein, the term “administering” means providing an agent to a subject in need thereof, and includes, but is not limited to, administering by a medical professional and self-administering. In some embodiments, without limitation, the methods described herein can be administered intravenously; intra-arterially; subcutaneously; intramuscularly; intraperitoneally; stereotactically; intranasally; mucosally; intravitreally; intrastriatally; or intrathecally. The foregoing administration routes can be accomplished via implantable microbead (e.g., microspheres, sol-gel, hydrogels); injection; continuous infusion; localized perfusion; catheter; or by lavage. In some embodiments, the compositions and formulations of the present disclosure are administered via injection or infusion, preferably by intravenous, subcutaneous, or intra-arterial administration. Methods for administering a formulation of a FAIM or fragments and/or mimetic thereof can adapted from Remington’s Pharmaceutical Sciences (17th Ed., Mack Pub. Co. 1985), the disclosure of which is incorporated herein by reference in its entirety.

In various embodiments, methods are provided for the prevention and/or treatment of a neurodegenerative or other proteinopathy in a patient, comprising administering to the subject in need thereof, a therapeutically effective amount of one or more isolated FAIM proteins or a fragment and/or mimetic thereof. The methods contemplate administering one or more compositions that are pharmaceutically acceptable for the treatment of humans, particularly humans who have suffered a neurodegenerative or other proteinopathy, for example, any disease disclosed herein and are deemed safe and effective. In various embodiments, the administration of the one or more isolated FAIM proteins or fragments and/or mimetic thereof can be accomplished using an administration method known to those of ordinary skill in the art.

Dosages for a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability or serum half-life of the one or more isolated FAIM proteins or fragments and/or mimetic thereof employed and the condition of the patient, as well as the body weight or surface area of the patient to be treated. The size of the dose is also determined by the existence, nature, and extent of any adverse side-effects that accompany the administration of a particular vector, formulation, or the like in a particular patient.

“Dosage unit” means a form in which a pharmaceutical agent or agents are provided, e.g. a solution or other dosage unit known in the art. Further, as used herein, “Dose” means a specified quantity of a pharmaceutical agent provided in a single administration, or in a specified time period. In certain embodiments, a dose can be administered in one, two, or more, boluses, infusions, or injections. For example, in certain embodiments where intravenous or subcutaneous administration is desired, the desired dose may require a volume not easily accommodated by a single injection, therefore, two or more injections can be used to achieve the desired dose, or one or more infusions are administered. In certain embodiments, the pharmaceutical agent is administered by infusion over an extended period of time or continuously. Doses can be stated as the amount of pharmaceutical agent per hour, day, week, or month. Doses can be expressed as µg/kg, mg/kg, g/kg, mg/m2 of surface area of the patient.

Therapeutic compositions comprising one or more isolated FAIM proteins or fragments and/or mimetic thereof are optionally tested in one or more appropriate in vitro and/or in vivo animal models of disease, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the EC₅₀ of the relevant formulation, and/or observation of any side-effects of the one or more isolated FAIM proteins or fragments and/or mimetic thereof at various concentrations, e.g., as applied to the mass and overall health of the patient. Administration can be accomplished via single or divided doses. Various factors may be used by a skilled practitioner, for example, a clinician, physician, or medical specialist to properly administer one or more isolated FAIM proteins or fragments and/or mimetic thereof. For example, if using a composition containing one or more isolated FAIM proteins or fragments and/or mimetic thereof that can circulate freely in the bloodstream, the composition or formulation may be administered intravenously; intra-arterially; subcutaneously; intramuscularly; intraperitoneally; stereotactically; intranasally; mucosally; intravitreally; intrastriatally; or intrathecally. In some embodiments, the one or more isolated FAIM proteins or fragments and/or mimetic thereof may be administered prior to, concomitantly with or subsequent to the administration of a secondary active agent.

In some embodiments, a first dose of one or more isolated FAIM proteins or fragments and/or mimetic thereof is administered as an intravenous bolus, followed by subsequent doses by infusion or injection as maintenance doses. The one or more isolated FAIM proteins or fragments and/or mimetic thereof can be administered in various ways; for example, the one or more isolated FAIM proteins or fragments and/or mimetic thereof can be administered alone, or as an active ingredient in combination with pharmaceutically acceptable carriers, diluents, adjuvants and vehicles, or in concert with another medicament commonly prescribed for use in patients with a neurodegenerative proteinopathy. The one or more isolated FAIM proteins or fragments and/or mimetic thereof can be administered parenterally, for example, intravenously, intra-arterially, subcutaneously administration as well as intrathecal and infusion techniques, or by local administration or direct administration (stereotactic administration) to the site of disease or pathological condition, for example, in the appropriate region of the brain. Repetitive administrations of the one or more isolated FAIM proteins or fragments and/or mimetic thereof may also be useful, where short term or long term (for example, hours, days or weeklong administration) is desirable. In various embodiments, one or more isolated FAIM proteins or fragments and/or mimetic thereof may be administered parenterally, preferably by intravenous administration either by direct injection, infusion or via catheter administration as approved for the treatment of one or more of the neurodegenerative proteinopathies by regulatory review by a competent regulatory body, for example, the US Food and Drug Administration (FDA) or the European Medicines Agency.

The subject or patient being treated is a warm-blooded animal and, in particular, mammals, including humans. The pharmaceutically acceptable carriers, diluents, adjuvants and vehicles as well as implant carriers generally refer to inert, non-toxic solid or liquid fillers, diluents or encapsulating material not reacting with the active components of the invention.

“Mammal” or “mammalian” refers to a human or non-human mammal, including, but not limited to, mice, rats, rabbits, dogs, cats, pigs, and non-human primates, including, but not limited to, monkeys and chimpanzees.

In some embodiments, when administering one or more isolated FAIM proteins or fragments and/or mimetic thereof parenterally, it will generally be formulated in a unit dosage injectable form (for example, in the form of a liquid, for example, a solution, a suspension, or an emulsion). Some pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

A pharmacological formulation of some embodiments may be administered to the patient in an injectable formulation containing any compatible carrier, such as various vehicle, adjuvants, additives, and diluents; or the inhibitor(s) utilized in some embodiments may be administered parenterally to the patient in the form of slow-release subcutaneous implants or vectored delivery, iontophoretic, polymer matrices, liposomes, and microspheres. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the one or more isolated FAIM proteins or fragments and/or mimetic thereof. In addition, a pump-based hardware delivery system may be used to deliver one or more embodiments.

Examples of systems in which release occurs in bursts includes, e.g., systems in which the one or more isolated FAIM proteins or fragments and/or mimetic thereof, is entrapped in liposomes which are encapsulated in a polymer matrix, wherein the liposomes are sensitive to specific stimuli, e.g., temperature, pH, light, and/or other degrading stimuli, and burst release occurs accordingly when the system in confronted with one of the aforementioned stimuli. Many other such implants, delivery systems, and modules are well known to those skilled in the art.

In some embodiments, without limitation, one or more isolated FAIM proteins or fragments and/or mimetic thereof may be administered initially by an infusion or intravenous injection to bring blood levels of one or more isolated FAIM proteins or fragments and/or mimetic thereof to a suitable level. The patient’s levels are then maintained by an intravenous dosage form of the one or more isolated FAIM proteins or fragments and/or mimetic thereof, although other forms of administration, dependent upon the patient’s condition and as indicated above, can be used. The quantity to be administered and timing of administration may vary for the patient being treated.

Additionally, in some embodiments, without limitation, one or more isolated FAIM proteins or fragments and/or mimetic thereof may be administered in situ to bring internal levels to a suitable level. The patient’s levels are then maintained as appropriate in accordance with good medical practice by appropriate forms of administration, dependent upon the patient’s condition. The quantity to be administered and timing of administration may vary for the patient being treated.

In certain non-limiting embodiments, one or more isolated FAIM proteins or fragments and/or mimetic are administered via intravenous injection, for example, a subject is injected intravenously with a formulation of one or more isolated FAIM proteins or fragments and/or mimetic thereof suspended in a suitable carrier using a needle with a gauge ranging from about 7-gauge to 25-gauge (see Banga (2015) Therapeutic Peptides and Proteins: Formulation, Processing, and Delivery Systems; CRC Press, Boca Raton, FL). An illustrative example of intravenously dosed FAIM proteins or fragments and/or mimetic thereof includes, but is not limited to, uncovering the injection site; determining a suitable vein for injection; applying a tourniquet and waiting for the vein to swell; disinfecting the skin; pulling the skin taut in the longitudinal direction to stabilize the vein; inserting needle at an angle of about 35 degrees; puncturing the skin, and advancing the needle into the vein at a depth suitable for the subject and/or location of the vein; holding the injection means (e.g., syringe) steady; aspirating slightly; loosening the tourniquet; slowly injecting the one or more FAIM proteins or fragments and/or mimetic thereof, checking for pain, swelling, and/or hematoma; withdrawing the injection means; and applying sterile cotton wool onto the opening, and securing the cotton wool with adhesive tape (alternatively, and bandage or other means to cover the injection site may be used).

In some embodiments, the initial administration may include an infusion of one or more isolated FAIM proteins or fragments and/or mimetic thereof via intravenous administration over a period of 1 minute to 120 minutes. Subsequent doses of the one or more isolated FAIM proteins or fragments and/or mimetic thereof can be accomplished using intravenous injections or by infusion. Each dose administered may be therapeutically effective doses or suboptimal doses repeated if needed.

Any appropriate routes of isolated FAIM proteins or fragments and/or mimetic thereof administration known to those of ordinary skill in the art may comprise embodiments of the invention. One or more isolated FAIM proteins or fragments and/or mimetic thereof, can be administered and dosed in accordance with good medical practice, taking into account the clinical condition of the individual patient, the site and method of administration, scheduling of administration, patient age, sex, body weight, body mass index (BMI), surface area (e.g., in the context of chemotherapy calculations), and other factors known to medical practitioners.

In some embodiments, the administration is designed to supply the one or more isolated FAIM proteins or fragments and/or mimetic thereof to the brain tissue that requires the effects provided by the one or more isolated FAIM proteins or fragments and/or mimetic thereof to dissolve, disaggregate or solubilize protein aggregates in the brain of the subject being treated. In some embodiments, the target tissue includes one or more of: the blood vessels of the subject, the blood vessels of the brain and brain tissue.

For example, in one embodiment, a dose of the one or more isolated FAIM proteins or fragments and/or mimetic thereof may include administration of about 0.01 mg/kg (wt/wt%) to about 100 mg/kg (wt/wt%) of the weight of the patient administered per dose, one or more times per day, or one or more times per week, or one or more times per month. In certain embodiments, a dosage unit of one or more isolated FAIM proteins or fragments and/or mimetic thereof is a vial containing 0.01 mg/kg (wt/wt%) to about 100 mg/kg (wt/wt%) of the weight of the patient and at least one pharmaceutically acceptable excipient. In some embodiments, a specific daily dose of one or more isolated FAIM proteins or fragments and/or mimetic thereof can include from about 500 µg to about 500 mg, or from about 750 µg to about 300 mg, or from about 1 mg to about 200 mg, or from about 10 mg to about 150 mg administered per dose or divided doses per day. In some embodiments, a therapeutically effective dose is a daily dose of about 500 µg to about 500 mg, or from about 750 µg to about 300 mg, or from about 1 mg to about 200 mg, or from about 10 mg to about 150 mg.

In certain embodiments, a dosage unit of one or more isolated FAIM proteins or a fragment or fragments and/or mimetic thereof is a vial containing 0.01 µg/kg (wt/wt%) to about 100 mg/kg (wt/wt%) of the weight of the patient and at least one pharmaceutically acceptable excipient. In some embodiments, a specific daily dose of one or more isolated FAIM proteins or fragments and/or mimetic thereof can include from about 0.5 µg to about 500 mg, or from 0.6 µg to about 250 mg, or from about 1 µg to about 100 mg, or from 0.5 µg to about 1 mg, or from about 1 µg to about 1 mg, or from 0.5 µg to about 50 µg, or from about 1.5 µg to about 500 µg, or from about 1.5 µg to about 100 µg, or from about 2 µg to about 50 µg, or from about 2 µg to about 30 µg, or from about 2 µg to about 20 µg, or from about 2 µg to about 16 µg administered per dose or divided doses per day. In some embodiments, a therapeutically effective dose is a daily dose of about 0.5 µg to about 500 mg, or from 0.6 µg to about 250 mg, or from about 1 µg to about 100 mg, or from about 1 µg to about 1 mg, or from about 1.5 µg to about 500 µg, or from about 1.5 µg to about 100 µg, or from about 2 µg to about 50 µg, or from about 2 µg to about 30 µg, or from about 2 µg to about 20 µg, or from about 2 µg to about 16 µg. In any embodiment, the total daily dose may be divided doses, the first dose administered as an initial bolus and the remainder infused over a period of time ranging from about 5 minutes to about 120 minutes. In some embodiments, the one or more isolated FAIM proteins or fragments and/or mimetic thereof is administered in a therapeutically effective amount of about 500 µg to about 500 mg, or from about 750 µg to about 300 mg, or from about 1 mg to about 200 mg, or from about 10 mg to about 150 mg, for example, one or more doses dosed daily, one or more times per day, one or more times per week or one or more times per month for one week to 12 months after the initial diagnosis of the neurodegenerative proteinopathy.

In any embodiment, the composition including one or more FAIM proteins or fragments and/or mimetics thereof may have a concentration from about 0.5 µM to about 500 mM, or from 0.5 µM to about 250 mM, or from 0.5 µM to about 100 mM, or from 0.5 µM to about 1 mM, or from 0.5 µM to about 500 µM, or from 0.5 µM to about 100 µM, or from 0.5 µM to about 50 µM, or from 0.6 µg to about 250 µM, or from about 1 µg to about 100 mM, or from 0.5 µM to about 1 mM, or from about 1 µM to about 1 mM, or from about 1.5 µM to about 500 µM, or from about 1.5 µM to about 100 µM, or from about 2 µM to about 50 µM, or from about 2 µM to about 30 µM, or from about 2 µM to about 20 µM, or from about 2 µM to about 16 µM.

The examples herein are provided to illustrate advantages of the present technology and to further assist a person of ordinary skill in the art with preparing or using the compounds of the present technology or salts, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### Example 1 - Methods

### Reagents and Antibodies

Antibodies were generated and/or obtained pursuant to the following methods: Goat anti-HSP27 (M-20) and mouse anti-αA-Crystallin (B-2) antibodies were obtained from Santa Cruz Biotechnology®. Rabbit anti-vimentin, rabbit anti-histone H3, rabbit anti-MEK½, rabbit anti-HSP40, rabbit anti-HSP60, rabbit anti-HSP70, rabbit anti-HSP90, rabbit anti-AIF, rabbit anti-GFP, rabbit anti-ubiquitin, mouse anti-α-tubulin, rabbit anti-β-amyloid, rabbit anti-SOD1, goat anti-rabbit IgG-HRP-linked and horse anti-mouse IgG-HRP-linked antibodies were obtained from Cell Signaling Technology®. Mouse anti-FLAG (M2) antibody and mouse anti-β-actin antibody were obtained from Sigma-Aldrich®. Rabbit αB-Crystallin antibody was obtained from Enzo Life Sciences®. Mouse anti-ubiquitin (UB-1) was obtained from Abcam®. Rabbit anti-α-synuclein antibody was obtained from ThermoFisher Scientific®. Affinity purified anti-FAIM antibody was obtained from rabbits immunized with a peptide having the amino acid sequence “CYIKAVSSRKRKEGIIHTLI” (SEQ ID NO:5), which is a peptide sequence located near the C-terminal region of FAIM. An exemplary method of immunizing rabbits to obtain anti-FAIM antibody is disclosed in Kaku and Rothstein, Fas apoptosis inhibitory molecule expression in B cells is regulated through IRF4 in a feed-forward mechanism. J Immunol. 2009 Nov 1;183(9):5575-81, the disclosure of which is incorporated herein by reference in its entirety. Plasmids

pEGFP-C1 and pEGFP-N1 vectors, and a pCMV-DYKDDDDK (FLAG) (SEQ ID NO: 19) vector set, were obtained from Clontech®. Mutant constructs were prepared using the Advantage 2 PCR kit (Clontech®) and the Phusion Site-Directed Mutagenesis Kit (ThermoFisher Scientific®). Primers used for the cloning and the mutagenesis are shown in Table 1. The insert was verified by sequencing (Genewiz®).

TABLE 1

Description of primers and primer sequences

| Description/Name | Primer Type/Sequence | Primer Type/Sequence |
|---|---|---|
| Gene cloning | Forward primer (5′- 3′) | Reverse primer (3′- 5′) |
| FAIM-S into pcDNA3.3 | AATATGGCATCTGGAGATGACAGTC (SEQ ID NO: 20) | TTAACTTGCAATCTCTGGGATTTC (SEQ ID NO: 21) |
| FAIM-L into pcDNA3.3 | AATATGACAGATCTCGTAGCTGTTTGGG (SEQ ID NO: 22) | TTAACTTGCAATCTCTGGGATTTC (SEQ ID NO: 21) |
| FAIM-S into pCMV-HA and pCMV-(DYKDDDDK (SEQ ID NO: 19))-N | ATATAGAATTCATATGGCATCTGGAGATGACAGTC (SEQ ID NO: 23) | TATATCTCGAGTTAACTTGCAATCTCTGGGATTTC (SEQ ID NO: 24) |
| FAIM-L into pCMV-HA and pCMV-(DYKDDDDK (SEQ ID NO: 19))-N | ATATAGAATTCATATGACAGATCTCGTAGCTGTTTGGG (SEQ ID NO: 25) | TATATCTCGAGTTAACTTGCAATCTCTGGGATTTC (SEQ ID NO: 24) |
| FAIM-S into pCMV-(DYKDDDDK (SEQ ID NO: 19))-C | ATATAGAATTCTAATGACAGATCTCGTAGCTGTTTGG (SEQ ID NO: 26) | ATGGTACCACTTGCAATCTCTGGGATTTCT (SEQ ID NO: 27) |
| FAIM-L into pCMV-(DYKDDDDK (SEQ ID NO: 19))-C | ATATAGAATTCTAATGGCATCTGGAGATGACAGTCCTA (SEQ ID NO: 28) | ATGGTACCACTTGCAATCTCTGGGATTTCT (SEQ ID NO: 27) |
| FAIM-S into pTrcHis TA vector | ATGACAGATCTCGTAGCTGTTTGG (SEQ ID NO: 29) | TTAACTTGCAATCTCTGGGATTTC (SEQ ID NO: 21) |
| FAIM-L into pTrcHis TA vector | ATGGCATCTGGAGATGACAGTC (SEQ ID NO: 30) | TTAACTTGCAATCTCTGGGATTTC (SEQ ID NO: 21) |
| HSP27 into pTrcHis TA vector | ATGACCGAGCGCCGCGTCCCCTT (SEQ ID NO: 31) | TTACTTGGCGGCAGTCTCATCGGAT (SEQ ID NO: 32) |
| αB-crystallin into pTrcHis TA vector | ATGGACATCGCCATCCACCA (SEQ ID NO: 33) | CTATTTCTTGGGGGCTGCGGT (SEQ ID NO: 34) |
| α-synuclein A53T into pTrcHis TA vector | ATGGATGTATTCATGAAAGGACTTTC (SEQ ID NO: 35) | TTAGGCTTCAGGTTCGTAGTCTT (SEQ ID NO: 36) |
| SOD1 G93A into pTrcHis TA vector | ATGGCGACGAAGGCCGTGTG(SEQ ID NO: 37) | TTATTGGGCGATCCCAATTACAC (SEQ ID NO: 38) |
| mouse FAIM | CACCGTGACGGATCTCGTAGCTGTTTGG (SEQ ID NO: 39) | AAACAACAGCTACGAGATCCGTCAC (SEQ ID NO: 40) |
| human FAIM | CACCGACAGATCTCGTAGCTGTTTGGG (SEQ ID NO: 41) | AAACAAACAGCTACGAGATCTGTC (SEQ ID NO: 42) |
| sequence primer for pX-458 | TGGACTATCATATGCTTACCGTAACTTGAAAG (SEQ ID NO: 43) | |
| WT allele | ACG GAT CTC GTA GCT GTT TGG GAC G (SEQ ID NO: 44) | CCA GCG TGT ACT CGT ATG CGA AGC C (SEQ ID NO: 45) |
| knockout allele | CAG AAG AAC TCG TCA AGA AGG C (SEQ ID NO: 46) | CAA GCG AAA CAT CGC ATC GAG CG (SEQ ID NO: 47) |
| faim | TGGGTATTACACATGGATGGTG (SEQ ID NO: 48) | ACAAACTCACCCGCTGTCTC (SEQ ID NO: 49) |
| hspb5 | CAGCTGGTTTGACACTGGAC (SEQ ID NO: 50) | GGCGCTCTTCATGTTTTCCA (SEQ ID NO: 51) |
| hsp70 a1a | ACATGAAGCACTGGCCTTTC (SEQ ID NO: 52) | TCTCCTTCATCTTGGTCAGCAC (SEQ ID NO: 53) |
| hsp90 aa1 | TGGCAGCAAAGAAACACCTG (SEQ ID NO: 54) | CAGGAGCGCAGTTTCATAAAGC (SEQ ID NO: 55) |
| gapdh | CTGACTTCAACAGCGACACC (SEQ ID NO: 56) | GTGGTCCAGGGGTCTTACTC (SEQ ID NO: 57) |
| faim I/VxI/V #1 | CAAACGAGTAGTATATGgAGATGGAAAGG (SEQ ID NO: 58) | CCTGATGTAGTCCCATGTTCAAATT (SEQ ID NO: 59) |
| faim I/VxI/V #2 | AAAAGCGACCATAAATggAGACGCTATCA (SEQ ID NO: 60) | GTCTTTGCAGCTCCAACATAGAATG (SEQ ID NO: 61) |

The following plasmids were obtained from Addgene®.
EGFP-α-synuclein-A53T Cat. No. #40823
pEGFP-Q74, Cat. No. #40262
pEGFP-Q23, Cat. No. #40261
pF146 pSOD1WTAcGFP1, Cat. No. #26407
pF150 pSOD1G93AAcGFP1, Cat. sNo. #26411
pSpCas9(BB)-2A-GFP (PX458), Cat. No. #48138
FAIM expression vectors were constructed using pcDNA3.3 (Invitrogen®) (TA-cloning), pCMV-(DYKDDDDK (SEQ ID NO: 19))-C (Clontech®) (cloned into EcoRI and KpnI sites) and pCMV-HA vectors (Clontech®) (cloned into EcoRI and KpnI sites). Primers for gene cloning are shown in Table 1.

### Generation of FAIM-Deficient Mice

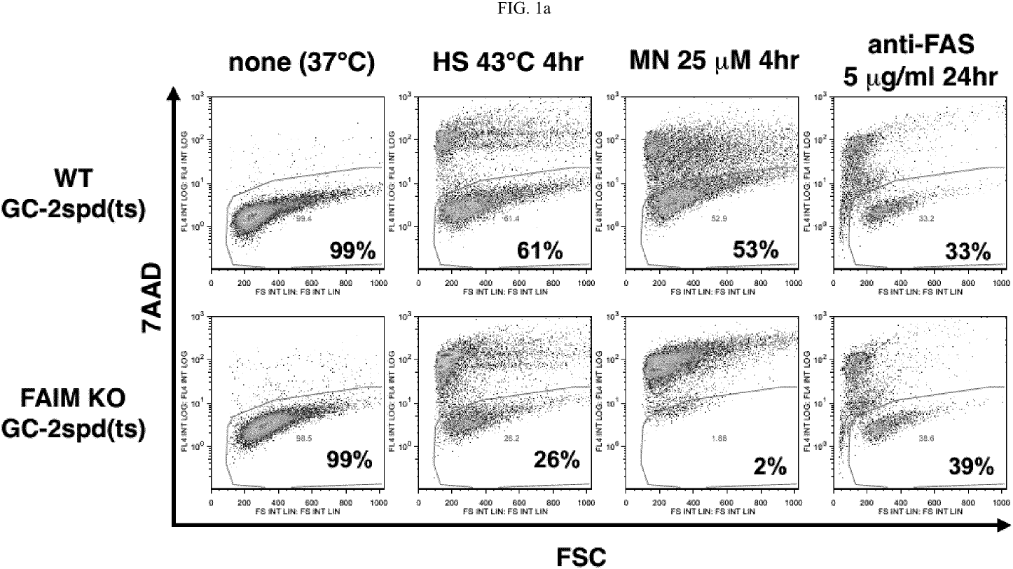

FAIM-deficient (KO) mice were generated in conjunction with the inGenious Targeting Laboratory®. The target region, including the FAIM coding regions of exons 3-6 (9.58 kb), was replaced by sequences encoding eGFP and neomycin-resistant genes (FIGS. 1a and 1b). The targeting construct was electroporated into ES cells derived from C57BL/6 mice. Positive clones were selected by neomycin and screened by PCR and then microinjected into foster C57BL/6 mice. Subsequent breeding with wild-type C57BL/6 mice produced F1 heterozygous pups. Offspring from heterozygous mice were selected using PCR. Mice were maintained on a C57BL/6 background. Next, genotyping PCR using genomic DNA from ear punches, was performed, using a mixture of four primers to identify the wild-type allele and the mutant alleles, generating 514bp and 389bp DNA amplicons, respectively. Primers are shown in Table 1. Mice were cared for and handled in accordance with National Institutes of Health and institutional guidelines. FAIM-KO mice were viable, developed normally and did not show any obvious phenotypic changes in steady state conditions (data not shown). The heterozygous intercrosses produced a normal Mendelian ratio of FAIM+/+, FAIM+/-, and FAIM-/- mice.

### Cell Culture and Transfection

HeLa, GC-1 spg, GC-2spd(ts) and HLE B-3 cell lines were obtained from the American Type Culture Collection (ATCC). HeLa cells were cultured in DMEM medium (Corning®) whereas GC-2spd(ts) and HLE B-3 cells were cultured in EMEM (Corning®). Both DMEM and EMEM contained 10% FCS, 10 mM HEPES, pH 7.2, 2 mM L-glutamine and 0.1 mg/ml penicillin and streptomycin. Transfection was performed using Lipofectamine 2000 for GC2spd(ts) cells or Lipofectamine 3000 for HeLa cells, according to the manufacturer’s instructions (Invitrogen®). Primary fibroblasts were purified and cultured as previously described. Briefly, skin in the underarm area (1 cm x 1 cm) was harvested in PBS. The tissue was cut into 1 mm pieces. To extract cells, tissues were incubated at 37° C. with shaking in 0.14 Wunsch units/ml Liberase Blendzyme 3 (Sigma-Aldrich®) and 1 x antibiotic/antimycotic (ThermoFisher Scientific®) in DMEM/F12 medium (Corning®) for 30 to 90 min until the medium appeared “fuzzy” when observed. Tissues were then washed with medium three times, and cultured at 37° C. After 7 days, cells were cultured in EMEM containing 15% FBS plus penicillin/streptomycin for another 7 days. Cells obtained from the foregoing procedure were subsequently used for the experiments below.

### Generation of FAIM Knockout Cell Lines With CRISPR/Cas9

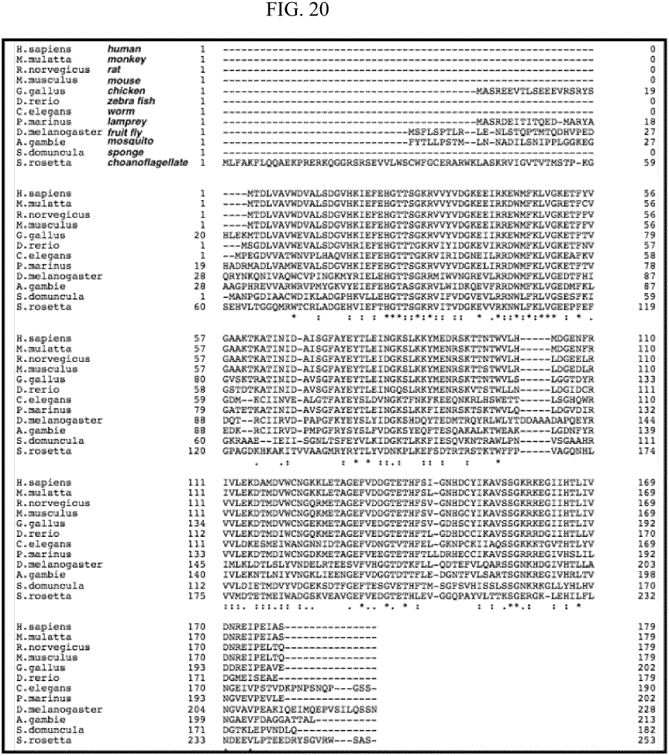

Guide RNA (gRNA) sequences for both human and mouse FAIM gene (FIG. 20) were designed using a CRISPR target design tool (http://crispr.mit.edu) in order to target the exon after the start codon. The designed DNA oligo nucleotides are shown in Table 1. Annealed double strand DNA sequences were ligated into pSpCas9(BB)-2A-GFP (PX458) vector (Addgene) at the Bpi1 (Bbs1) restriction enzyme sites using the “Golden Gate” cloning strategy. The presence of insert was verified by sequencing.

Empty vector was used as a negative control. Transfection was performed using lipofection and a week after the transfection, eGFP⁺ cells were sorted with an Influx instrument (Becton Dickinson), and seeded into 96 well plates. FAIM knockout clones were screened by limiting dilution and western blotting.

### Gene Expression Analysis by qPCR

Gene expression was assayed by real-time PCR. Briefly, RNA was prepared from cells using the RNeasy mini kit (Qiagen®), according to the manufacturer’s instructions. cDNA was prepared using iScript reverse transcription supermix (Bio-Rad®). Gene expression was then measured by real-time PCR using iTaq SYBR Green (Bio-Rad®) and normalized with GAPDH. Primer sequences are shown in Table 1.

### In Vitro Cellular Stress Induction

To induce mild heat shock, cells in culture dishes were incubated in a water bath at 43° C. for the indicated period. In some experiments, cells were recovered at 37° C. after heat stress induction at 43° C. for 2 hours or more as previously described. To induce oxidative stress, menadione (MN) (Sigma-Aldrich®), dissolved in DMSO at 100 mM, was added to medium at the indicated final concentration for 1 hour. In oxidative stress experiments where cells were harvested at time points beyond 1 hour with menadione, cells were washed once with medium and fresh medium (without menadione) was added to the cell culture as previously described. To induce FAS-mediated apoptosis in GC-2 spd (ts), cells were cultured with 5 µg/ml anti-FAS antibody (clone; Jo2, BD Pharmingen®) as previously described.

### In Vivo Mouse Stress Induction

Acute oxidative stress was induced by a single intraperitoneal injection of menadione (200 mg/kg in PBS) into mice. The mice were then euthanized 18 hours after the injection. Spleens and livers were removed and protein was immediately extracted for western blotting analysis.

### Cell Viability Analysis With Flow Cytometry

Adherent cells were detached by Trypsin-EDTA. Adherent and floating cells were harvested and pooled, after which cells were resuspended in 2 µg/ml 7-aminoactinomycin D (7-AAD) (Anaspec®). Cell viability was assessed using Gallios (Beckman Coulter®) or Attune (ThermoFisher Scientific) flow cytometers. Data were analyzed using FlowJo v9 or v10 software (TreeStar®).

### Viability Analysis by Released LDH Detection

Following stress induction in vitro or in vivo, LDH released into the supernatant, or into the serum, from damaged cells was quantified using the Cytotox 96 Non-radioactive Cytotoxicity Assay (Promega®). Serum samples were diluted in PBS (1:20).

### ALT Activity Assay

Following stress induction in vivo, serum was harvested and ALT levels were monitored using the ALT Activity Assay Kit (BioVision®). OD at 570 nm (colorimetric) was detected with a Synergy Neo2 instrument. Serum samples were diluted in ALT assay buffer (1:5).

### Western Blotting

Cells were washed twice with PBS and lysed in RIPA lysis buffer (1% Nonidet P-40, 0.5% sodium deoxycholate, 0.1% SDS, 150 mM NaCl, 50 mM Tris-HCl (pH 8.0), 2 mM EDTA) containing supplements of 2 mM Na3VO4, 20 mM NaF, and a protease inhibitor cocktail (Calbiochem®) for 30 min on ice. In addition to the above supplements, 10 mM N-ethylmaleimide (NEM) (Sigma-Aldrich®), 50 µM PR-619 (LifeSensors®) and 5 µM 1,10-phenanthroline (LifeSensors®) were added in the lysis buffer for ubiquitin detection by western blotting. Lysates were clarified by centrifugation at 21,100 x g for 10 min. Supernatants were used as RIPA-soluble fractions. The insoluble-pellets (the RIPA-insoluble fractions) were washed twice with RIPA buffer and proteins were extracted in 8 M urea in PBS. In some experiments, protein lysates were separated into 4 subcellular fractions (cytosolic, membrane/organelle, nucleic, and cytoskeletal/insoluble fractions) using ProteoExtract Subcellular Proteome Extraction Kit (Calbiochem®) according to the manufacturer’s instructions. Protein concentrations were determined using the 660 nm Protein Assay Reagent (Pierce®). Protein samples in 1 x Laemmli buffer with 2-ME were boiled for 5 min. Equal amounts of protein for each condition were subjected to SDS-PAGE followed by immunoblotting.

### Immunoprecipitation

Cells expressing FLAG-tag proteins were lysed in 0.4% Nonidet P-40, 150 mM NaCl, 50 mM Tris-HCl (pH 8.0), 2 mM EDTA, 2 mM Na3VO4, 50 mM NaF, and protease inhibitor cocktail for 30 min on ice. Lysates were clarified by centrifugation at 21,100 x g for 10 min. Equal amounts of protein for each supernatant were mixed with anti-FLAG M2 Magnetic Beads (Sigma-Aldrich®) and incubated at 4° C. under gentle rotation for 2 hr. Beads were washed with lysis buffer 4 times and FLAG-tag proteins were eluted with 100 µg/ml 3x FLAG peptide (Sigma-Aldrich®) 2 times. Eluates were pooled and western blotting was performed to detect FLAG-FAIM binding proteins.

### Filter Trap Assay (FTA)

WT and FAIM KO cells were transiently transfected with eGFP-tagged aggregation-prone protein expression vectors (huntingtin and SOD1), and fluorescently tagged cells were then harvested at 48 hours to detect protein aggregates. WT and FAIM KO cells were incubated with or without menadione then harvested after the indicated period to detect ubiquitinated protein aggregates. Cells were washed with PBS and then lysed in PBS containing 2% SDS, 1 mM MgCl2, protease inhibitor cocktail and 25 unit/ml Benzonase (Merck®). Protein concentrations were quantified using 660 nm Protein Assay Reagent with Ionic Detergent Compatibility Reagent (IDCR) (ThermoFisher Scientific®). Equal amounts of protein extracts underwent vacuum filtration through a pre-wet 0.2 µm pore size nitrocellulose membrane (GE Healthcare®) for the detection of ubiquitinated protein aggregates or a 0.2 µm pore size cellulose acetate membrane (GE Healthcare®) for the detection of huntingtin and SOD1 aggregates using a 96 well format Dot-Blot apparatus (Bio-Rad®). The membrane was washed twice with 0.1% SDS in PBS and western blotting using anti-ubiquitin or anti-GFP antibody was carried out to detect aggregated proteins. Cell free aggregates of β-amyloid (1-42), α-synuclein A53T and SOD1 G93A were similarly applied to nitrocellulose membrane.

### Pulse-Shape Analysis (PulSA)

Cells expressing eGFP-tagged huntingtin or SOD1 proteins were harvested and eGFP expression was analyzed on an LSR Fortessa (BD Pharmingen®) flow cytometer for PulSA analysis to detect protein aggregates, as previously described. Data was collected in pulse-area, height and width for each channel. At least 10,000 cells were analyzed.

### In Situ Proximity Ligation Assay (PLA)

Cells were cultured for 24 hours on poly-L-lysine coated coverslips (Corning®) in 24-well plates. Cells with or without cellular stress induction were fixed and permeabilized with ice-cold 100% methanol. PLA reaction was carried out according to the manufacturer’s instructions using Duolink In Situ Detection Reagents Orange (Sigma-Aldrich®). ProLong Gold Antifade Reagent with DAPI (Cell Signaling Technology®) was used to stain nuclei and to prevent fading of fluorescence. Fluorescence signals were visualized with a Nikon A1R+ confocal microscope (Nikon®).

### His-Tag Recombinant Protein Production

His-tag protein expression vectors were constructed using pTrcHis TA vector according to the manufacturer’s instructions. In brief, PCR amplified target genes were TA-cloned into the vector (Invitrogen®) and inserted DNA was verified by sequencing (Genewiz®). Proteins were expressed in TOP10 competent cells (Invitrogen®) and were purified using a Nuvia IMAC Nickel-charged column (Bio-Rad) on an NGC Quest chromatography system (Bio-Rad®). Protein purity was verified using TGX Stain-Free gels (Bio-Rad®) on ChemiDoc Touch Imaging System (Bio-Rad) and each protein was determined to be >90% pure.

### Thioflavin T Fluorescence Assay

Fibril/aggregation formation of 15 µM β-amyloid (1-42) (Anaspec®), purified 20 µM α-synuclein A53T, and SOD1-G93A was assessed by Thioflavin T (ThT) (Sigma-Aldrich®)fluorescence using a Synergy Neo2 Multi-Mode Microplate Reader (Bio-Tek®). Reader temperature was set at 37° C. with continuous shaking between reads. ThT fluorescence intensity was measured using an excitation wavelength of 440 nm and an emission of 482 nm. PMT gain was set at 75. Fluorescence measurements were made from the top of the plate, with the top being sealed with an adhesive plate sealer to prevent evaporation.

### Generation of Pre-Formed Protein Aggregates

All fibrils were assembled in an Eppendorf ThemoMixer F 1.5 with ThermoTop, as previously described, with minor modifications. β-amyloid (50 µM) fibrils were assembled in β-amyloid Assay buffer (Anaspec®) for 5 hours with agitation at 500 rpm at 37° C. SOD1 G93A (80 µM) and α-synuclein A53T (80 µM) fibrils were generated in assembly buffer (AB; 40 mM HEPES-KOH pH 7.4, 150 mM KCl, 20 mM MgCl2 and 1 mM DTT) plus 10% (v/v) glycerol for 16 hours with agitation. All fibrils were recovered by centrifugation, washed and resuspended in the original buffers for disaggregation assays. For all fibrils, generation was confirmed by ThT fluorescence. Fibrils were diluted to the requisite concentration for subsequent disaggregation reactions.

### Disaggregation Assay by ThT Fluorescence and Sedimentation Analysis

β-amyloid (1-42) (1 µM), SOD1 G93A (2 µM) and α-synuclein A53T (2 µM) pre-formed fibrils were incubated with 8 µM recombinant proteins at 37° C. for 2.5 hours. Then, fibril status was determined by either ThT fluorescence or by detecting proteins in the supernatants and in the pellets after sedimentation and western blotting.

### Statistics

All quantitative data are expressed as mean ± SEM. One-way ANOVA was used for statistical determinations with GraphPad Prism 7 software. Values of p<0.05 are considered statistically significant (*p<0.05, **p<0.01 or ***p<0.001).

### Example 2 - FAIM-Deficient Cells are Susceptible to Heat Shock and Oxidative Stress

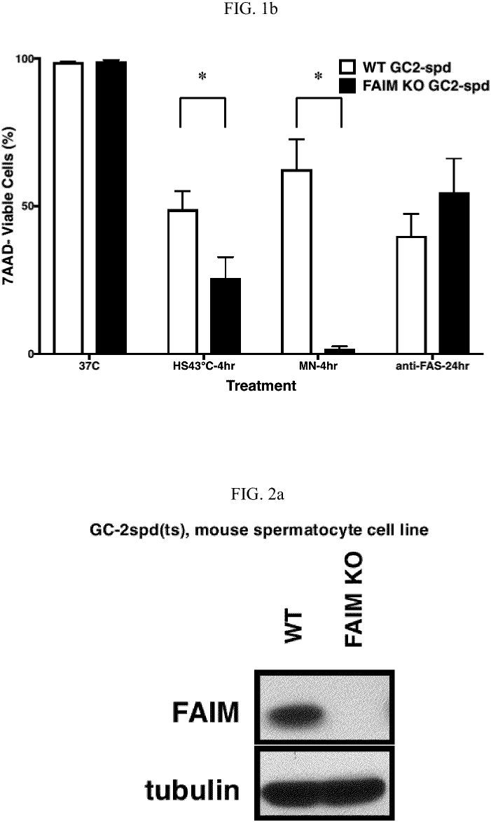

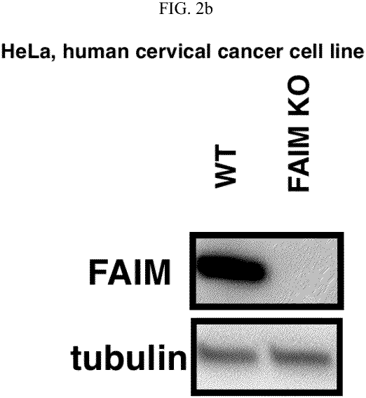

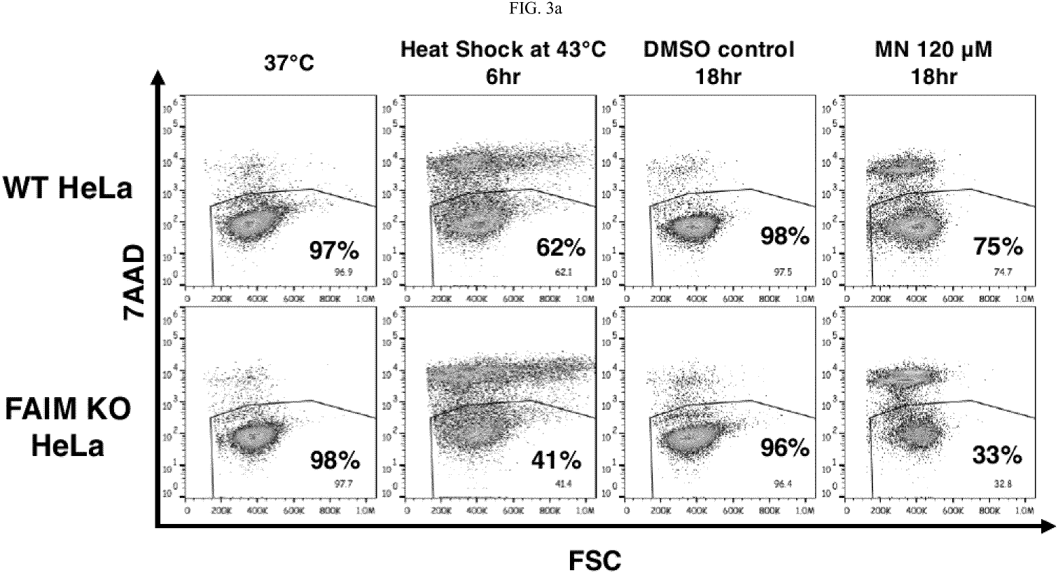

To test the activity of FAIM with respect to stress in testicular cells, we established a FAIM-deficient GC-2spd(ts) germ cell line by CRISPR-Cas9 excision and confirmed FAIM-deficiency by western blotting (FIG. 2a). FAIM KO and WT GC-2spd(ts) cells were cultured under stress conditions and cell viability was assayed by 7-AAD staining (FIG. 1a). Cell viability after heat shock and oxidative stress decreased in the absence of FAIM to a much greater extent than in FAIM-sufficient WT GC-2spd(ts) cells (FIGS. 1a and 1b). Here, there was not a significant difference in FAS-induced cell death (FIGS. 1a and 1b). Furthermore, similar results were obtained regarding diminished cell viability in stressed FAIM-deficient GC-1 spg germ cells (data not shown).

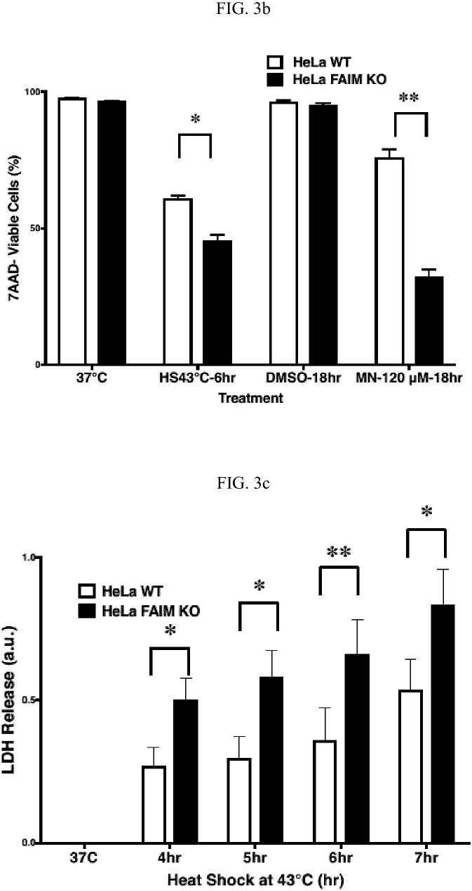

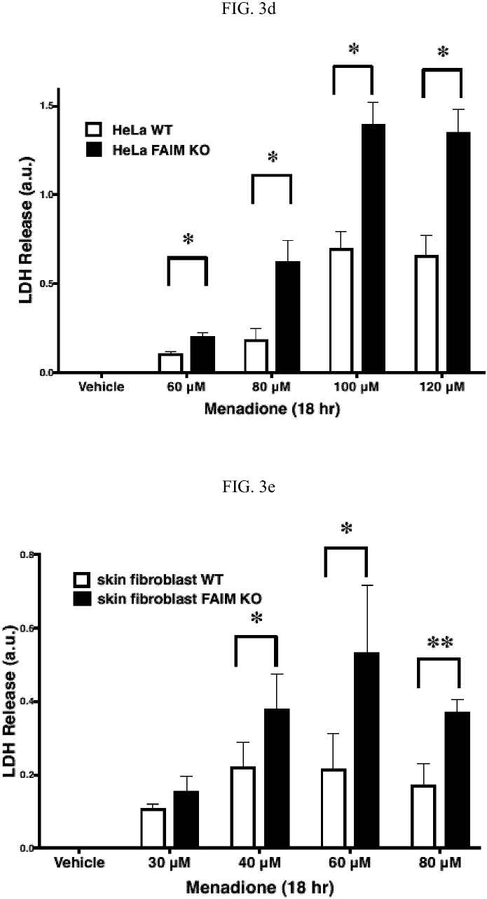

To exclude the possibility that FAIM protection is limited to germ cells, FAIM-deficient HeLa cells were generated with CRISPR-Cas9 (FIG. 2b). Similar to GC-2spd(ts) cells, FAIM-deficient HeLa cells were highly susceptible to stress-induced cell death (FIGS. 3a and 3b), to a greater extent than control HeLa cells.

HeLa cells (FIGS. 3a-3d) or mouse primary fibroblasts (FIGS. 3e and 3f), were incubated under stress conditions as noted for the indicated periods of time. WT HeLa cells and FAIM KO HeLa cells and fibroblasts from WT mice and from FAIM KO mice were stained with 7-AAD and cell viability was analyzed by flow cytometry after exposure to heat shock and menadione (MN)-induced oxidative stress. Representative flow data (FIG. 3a) and a summary of pooled data from 3 independent experiments (FIG. 3b) are shown. These results show that FAIM KO cells including FAIM KO primary cells are susceptible to heat/oxidative stress-induced cell death.

To confirm these results with high-throughput methodology, we measured supernatant levels of lactate dehydrogenase (LDH) released from cells upon stress induction and again we found increased cellular disruption in the face of FAIM deficiency (FIGS. 3c and 3d). Briefly, cell viability was determined by supernatant LDH leaked from WT HeLa cells and FAIM KO HeLa cells upon heat shock (FIG. 3c) or upon menadione-induced oxidative stress (FIG. 3d), as indicated. Here, FIG. 3c and FIG. 3d show the results of pooled data from 3 independent experiment.

FIGS. 3e and 3f depict the results of primary fibroblasts treated with either menadione or arsenite. Primary fibroblasts from WT and FAIM KO mice were subjected to menadione-induced (FIG. 3e) and arsenite-induced (FIG. 3f) oxidative stress in vitro, and cell viability was determined by supernatant LDH. Pooled data from 3 independent experiments are shown.

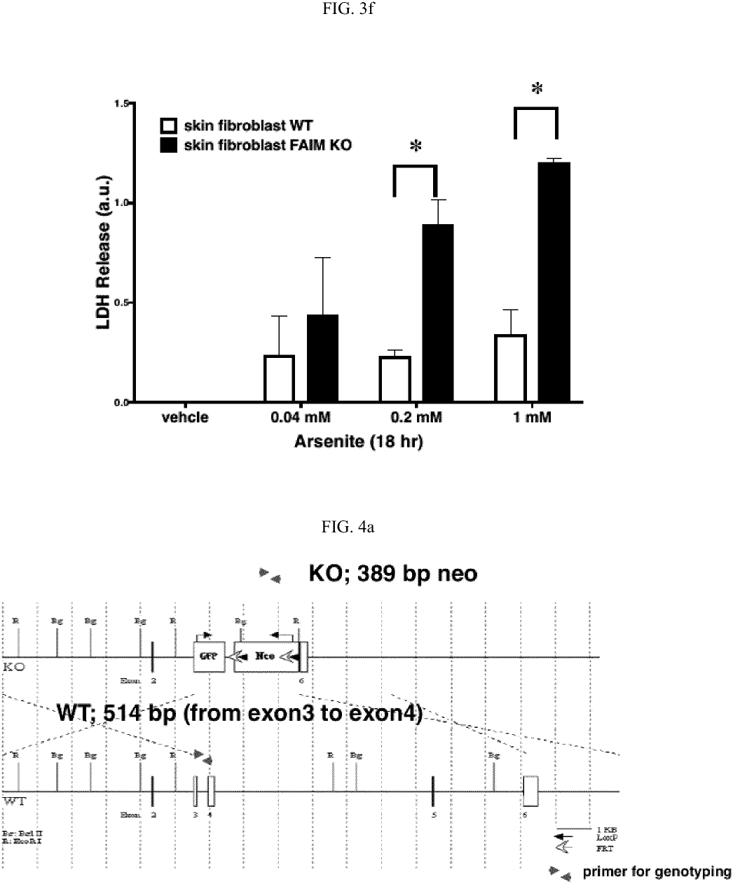

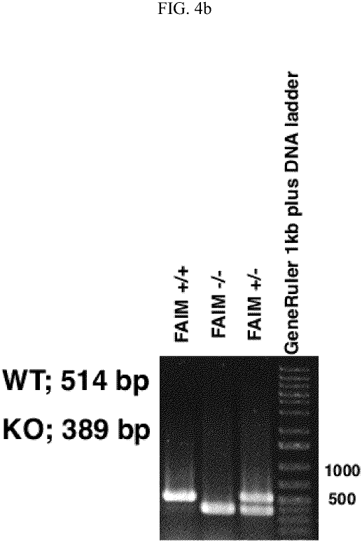

To validate the role of FAIM in primary cells, we developed FAIM KO mice in which the mouse FAIM gene was disrupted. Here, the results show that FAIM KO mice lack exons 3-5. FIG. 4a shows the schematic representation of the targeting vector and the targeted allele of the mouse FAIM gene. FIG. 4b shows the genotype determination of FAIM mice by PCR. Multiplex PCR genotyping analyses for KO (389 bp) and WT (514 bp) FAIM genes were performed to confirm the genotypes of wild-type (^{+/+}), heterozygous (^{+/-}) and homozygous (^{-/-}) mice. Representative genotyping results are shown.

Next, skin-derived fibroblasts were examined because these cells have been shown to be susceptible to menadione- and arsenite-induced oxidative stress. Consistent with the cell line results, we found vulnerability to oxidative stress induced by menadione (FIG. 3e) and by arsenite (FIG. 3f) to be much greater in FAIM-deficient primary fibroblasts as compared to control fibroblasts. Thus, data from 3 different cell types indicate that FAIM plays an essential role in protecting cells from heat and oxidative insults.

### Example 3 - ROS Generation and Apoptosis Induction During Cellular Stress Conditions is Normal in FAIM-Deficient Cells.

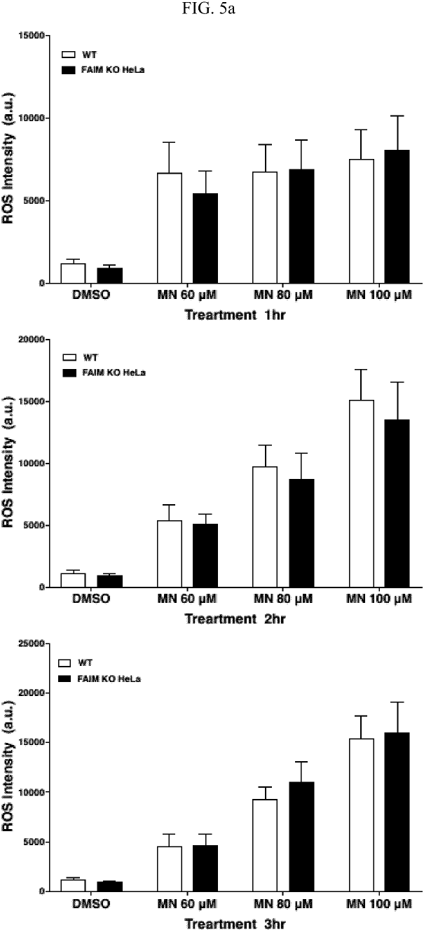

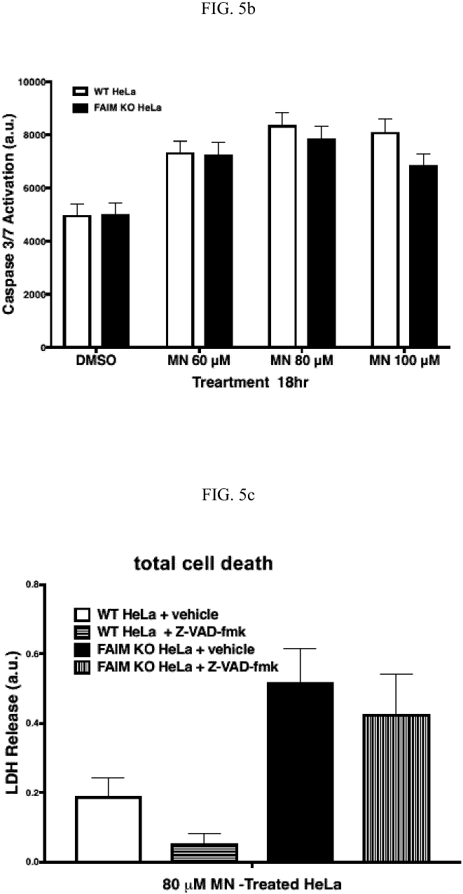

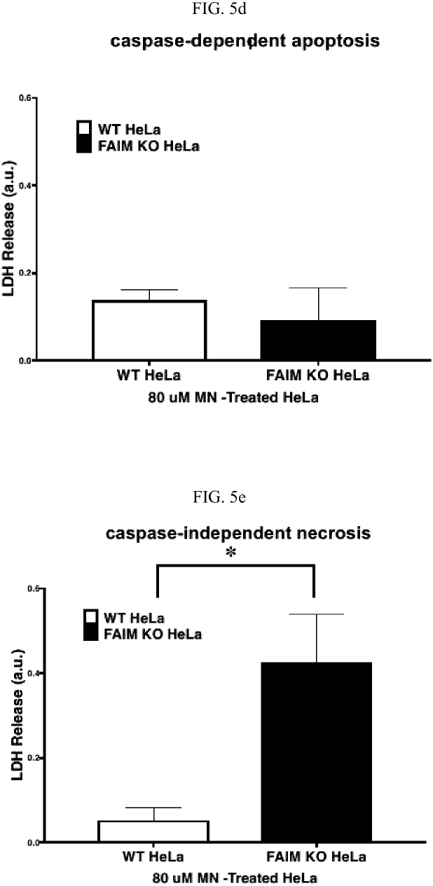

Oxidative stress and heat shock induce caspase-dependent apoptosis via ROS generation, which could play a role in stress-induced cell death that is affected by FAIM. To address this issue, we first evaluated ROS generation in FAIM-deficient and WT HeLa cells during oxidative stress, using the CellRox deep red staining reagent. We found no difference in stress-induced ROS, regardless of the presence or absence of FAIM (FIG. 5a). We then evaluated caspase activation under stress conditions, using the CellEvent caspase 3/7 detection reagent. We found that caspase 3/7 activity was not increased in FAIM-deficient HeLa cells (FIG. 5b).

To further evaluate stress-induced cell death, we separated cell death into caspase-dependent and caspase-independent forms. We pretreated cells with the pan-caspase inhibitor, Z-VAD-fmk peptide, before adding menadione, and then measured LDH release (FIG. 5c). Z-VAD-fmk has been reported to partially block menadione-induced cell death. However, we found menadione-induced LDH release was reduced to a small extent in both FAIM-deficient and FAIM-sufficient cells, resulting in similar levels of caspase-dependent apoptosis (FIG. 5d). Importantly, the increased LDH release induced by menadione in FAIM-deficient cells was for the most part resistant to caspase inhibition (FIG. 5e). Thus, menadione-induced cellular dysfunction, which is greatly magnified in the absence of FAIM, is largely caspase independent. In sum, there is no evidence that ROS/caspase-dependent apoptosis plays any role in the improved cellular viability produced by FAIM in the face of stress conditions.

### Example 4 - FAIM Protein Shifts to the Detergent-Insoluble Fraction During Stress

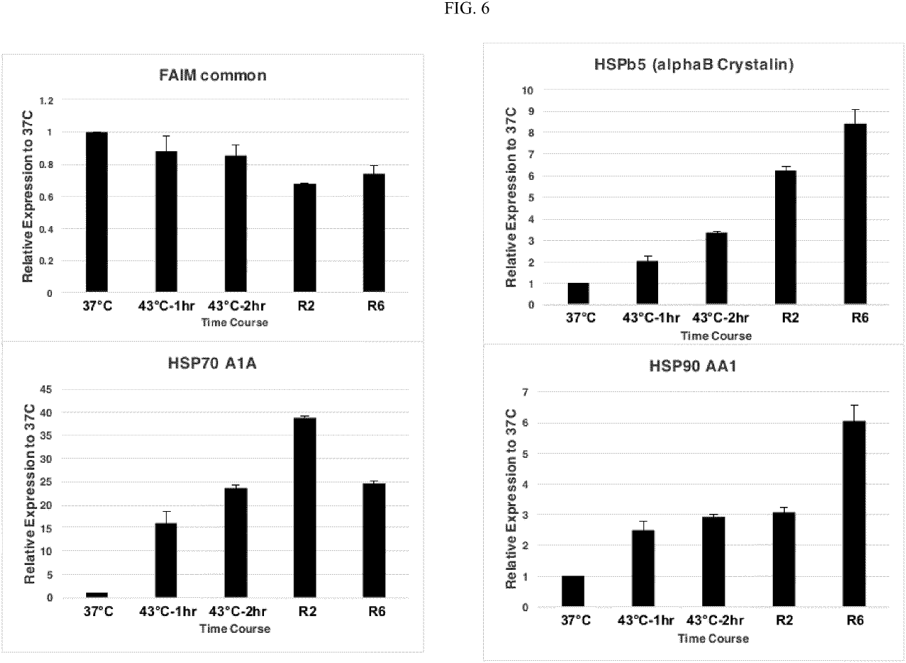

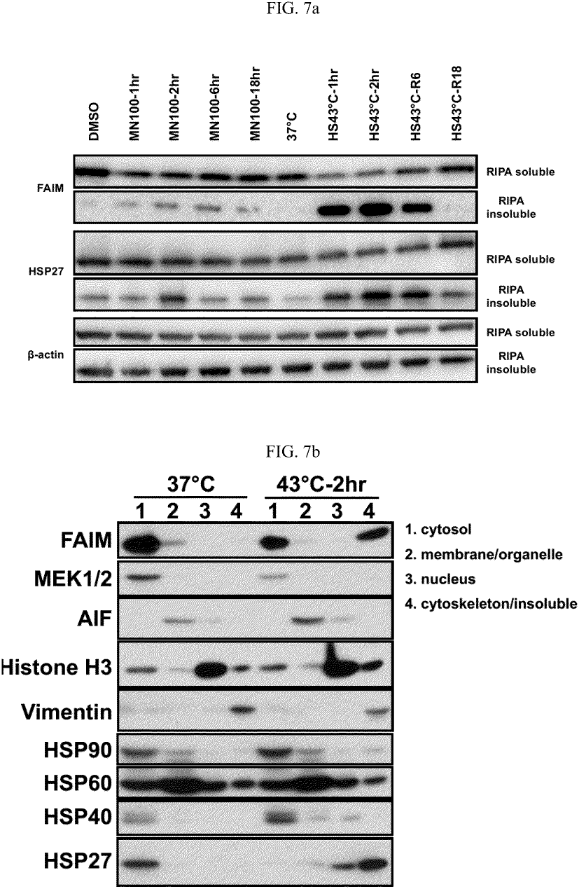

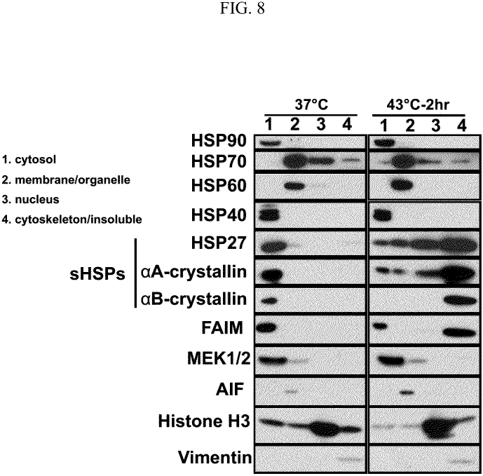

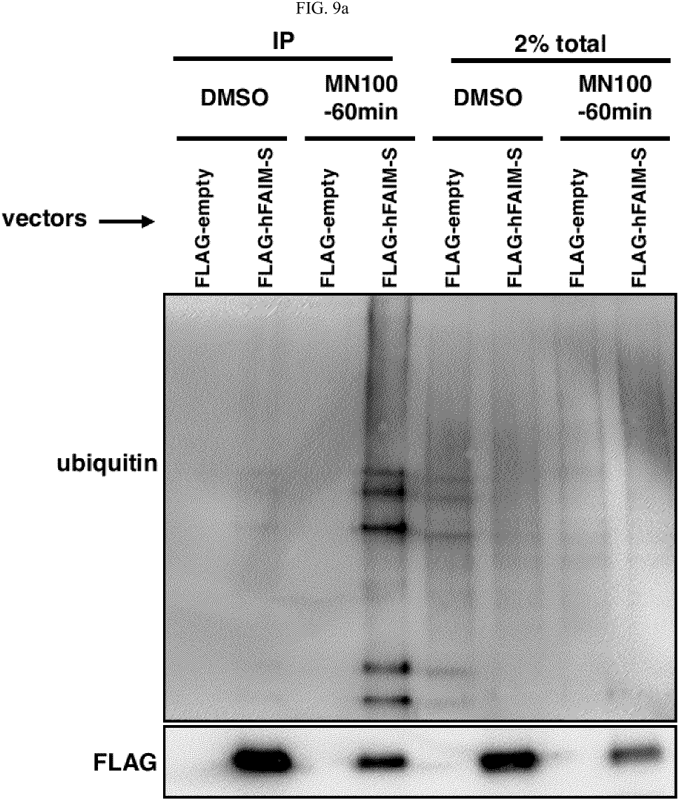

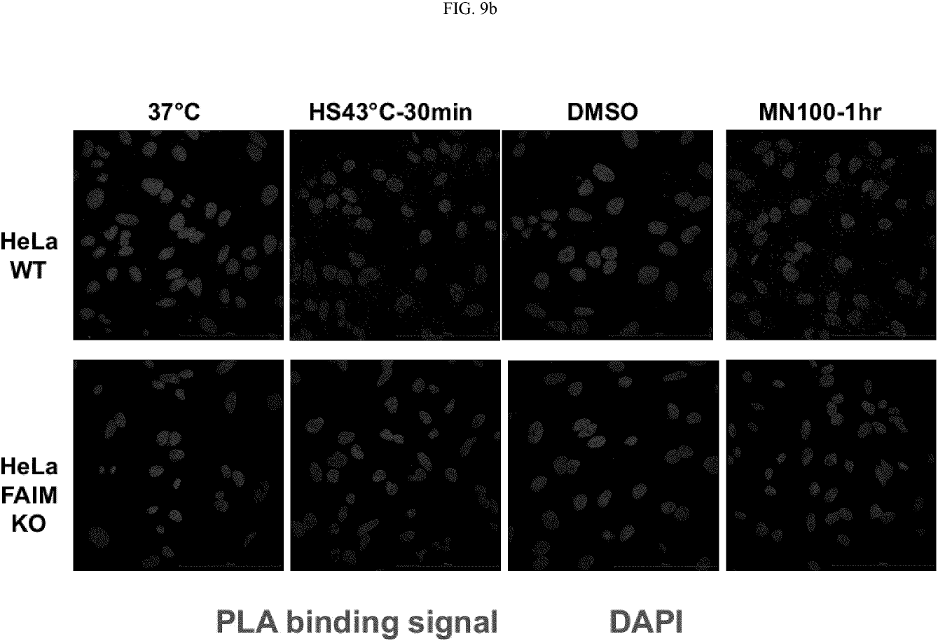

Heat shock proteins (HSPs) respond to stress conditions by upregulating expression. We examined FAIM expression in HeLa cells to determine if expression is upregulated by heat shock similar to HSPs. We found FAIM mRNA expression was not increased under stress conditions, in contrast to HSPs that were increased (FIG. 6), and further, FAIM protein expression levels were actually decreased in the RIPA lysis buffer soluble fraction (FIG. 7a). However, additional analysis determined that the majority of FAIM protein had shifted to the detergent-insoluble fraction in response to cellular stress (FIG. 7a), which was especially noticeable after heat shock. A similar shift to the insoluble fraction was also observed in HSP27 protein, one of the small HSPs, after stress (FIG. 7a).

To validate these results using a different extraction method, we separated proteins into 4 fractions--cytosol, membrane/organelle, nuclear, and cytoskeletal/detergent-insoluble--after mild heat stress. We found that the majority of FAIM protein migrated to the cytoskeletal/detergent insoluble fraction (FIG. 7b). Large HSPs such as HSP90, HSP60, and HSP40 maintained their original subcellular distribution after heat stress, whereas HSP27 showed a marked shift to the cytoskeletal/detergent insoluble fraction (FIG. 7b). Proteins from HLE B-3 cells, which express other sHSPs such as αA- and αB-crystallins in addition to HSP27, were similarly analyzed with respect to subcellular distribution before and after heat stress. Here again, HSP27 and related crystallin proteins migrated to the cytoskeletal/detergent insoluble fraction in response to heat stress, unlike other proteins (FIG. 8). Thus, the bulk of FAIM protein migrates to the detergent-insoluble fraction when cells are exposed to stress, as do small HSP proteins.

### Example 5 - FAIM Binds Ubiquitinated Proteins

Stress-induced cellular dysfunction is often associated with the appearance of disordered and dysfunctional proteins that must be disposed of to maintain cellular viability. Stress-induced disordered proteins are tagged with ubiquitin for intracellular degradation via the proteasome system and the autophagic pathway. If the load of stress-affected, ubiquitinated proteins exceeds the handling capacity of disposal mechanisms, these proteins may accumulate in an insoluble form. To determine whether the stress-induced migration of FAIM to detergent insoluble material is associated with binding to ubiquitinated proteins, we examined FAIM KO HeLa cells. FAIM KO HeLa cells were transfected with FLAG-tagged FAIM proteins and subjected to oxidative stress followed by anti-FLAG IP and western blotting for ubiquitin (FIG. 9a). Separately, FAIM KO HeLa cells were subjected to heat shock and oxidative stress followed by PLA to detect close proximity of FAIM and ubiquitin (FIG. 9b). Both Co-IP and PLA approaches demonstrated stress-induced interaction between FAIM and ubiquitinated protein. These data indicate that FAIM and ubiquitinated proteins associate with each other in response to cellular stress induction before becoming insoluble.

### Example 6 - Ubiquitinated Protein Aggregates Accumulate in FAIM-Deficient Cells Following Stress in Vitro.

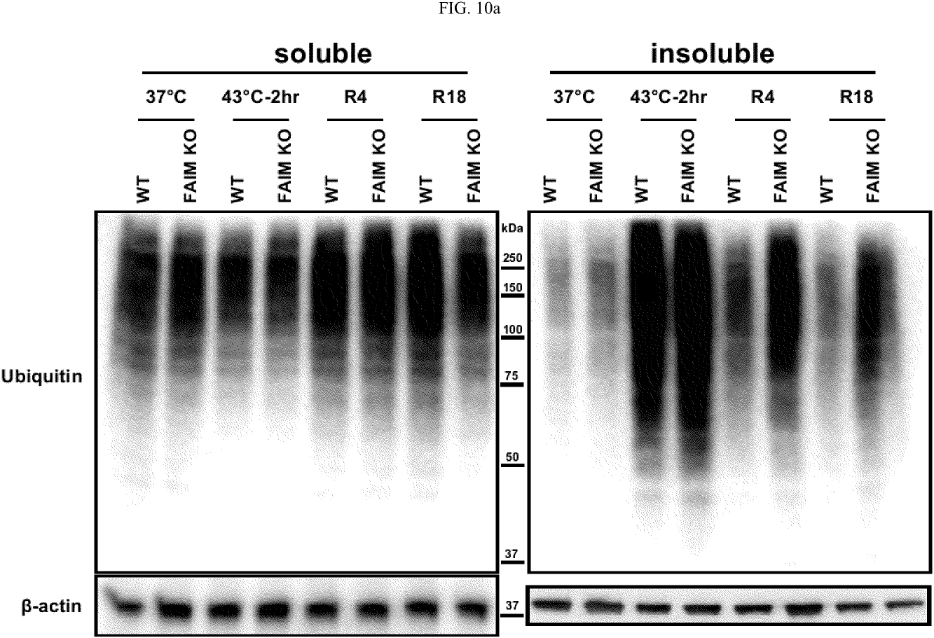

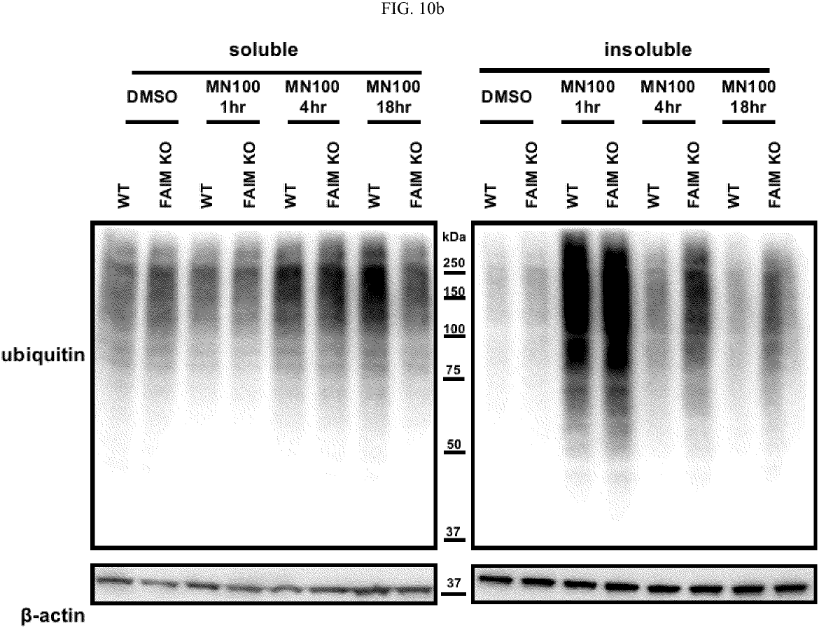

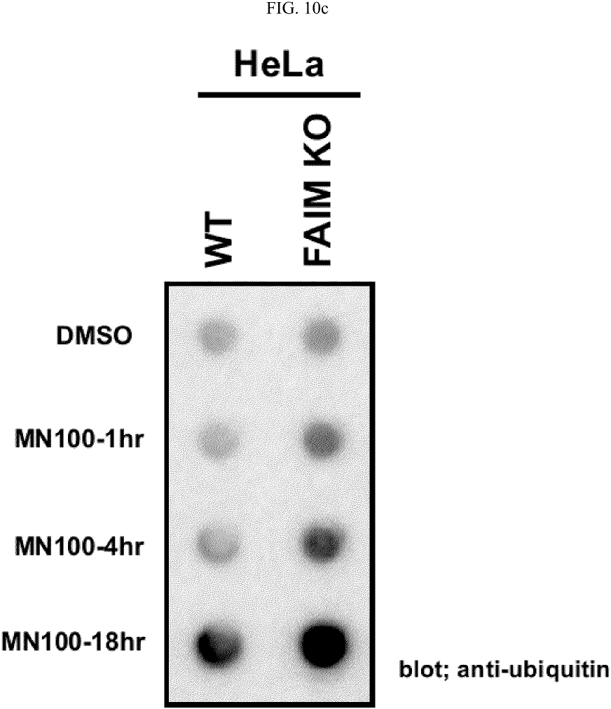

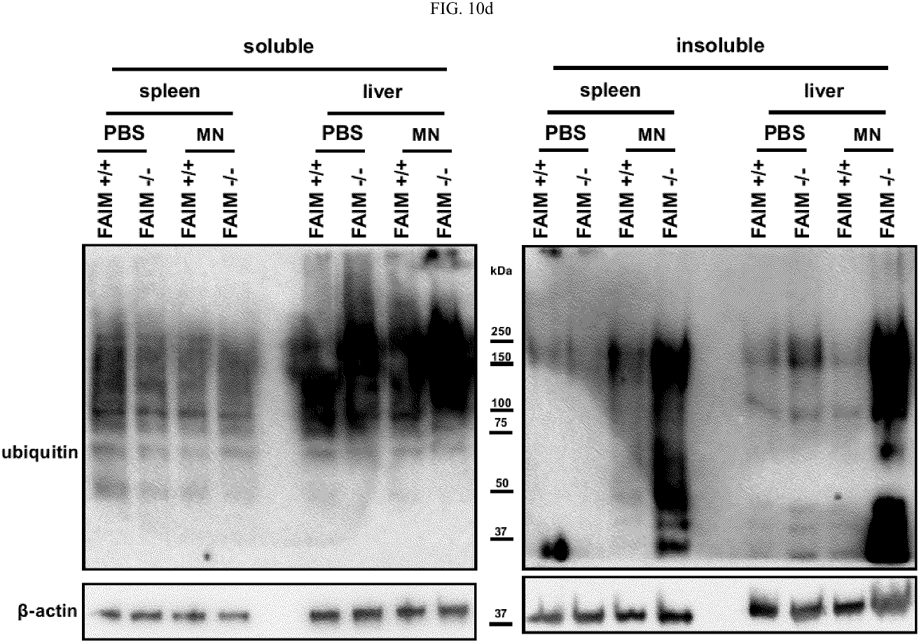

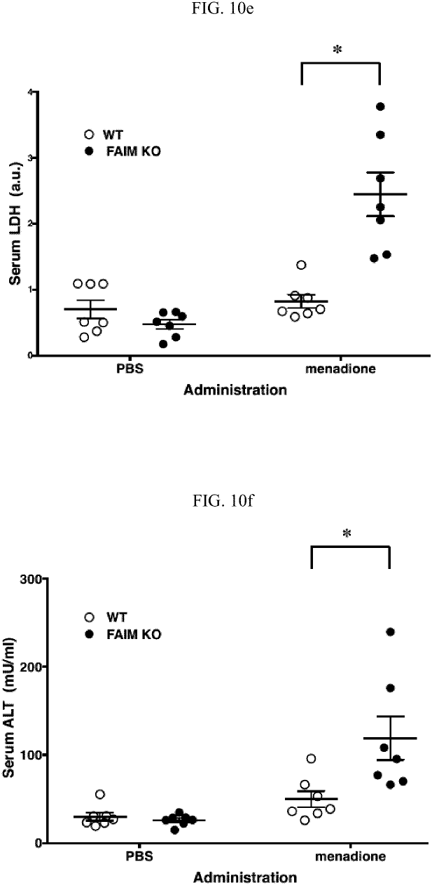

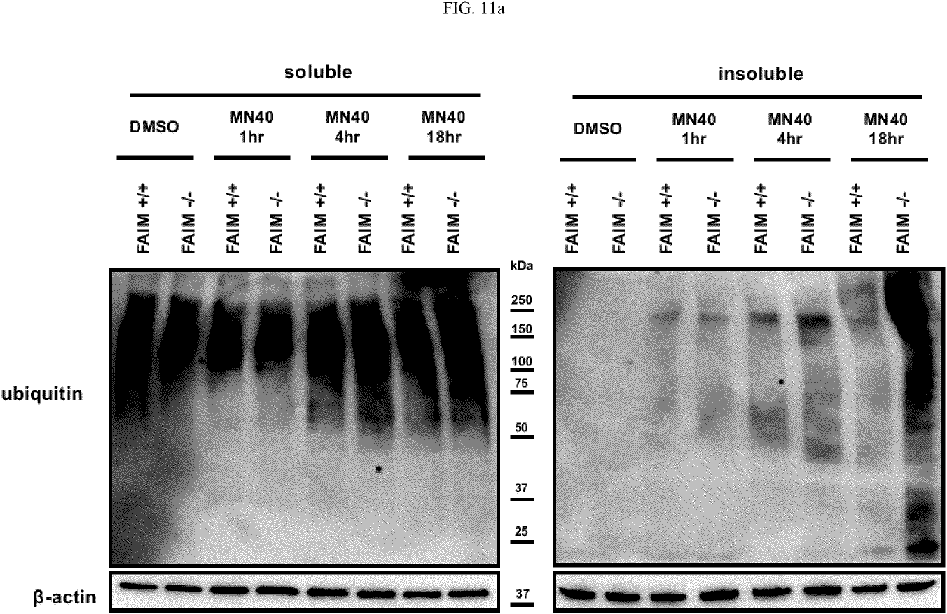

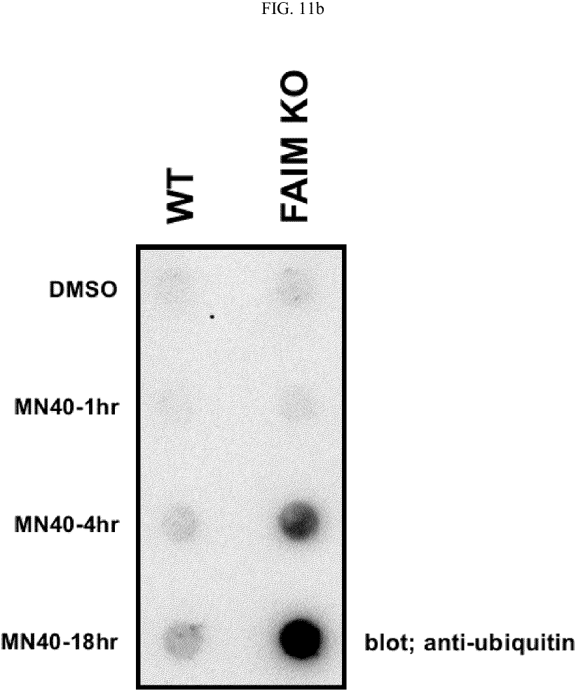

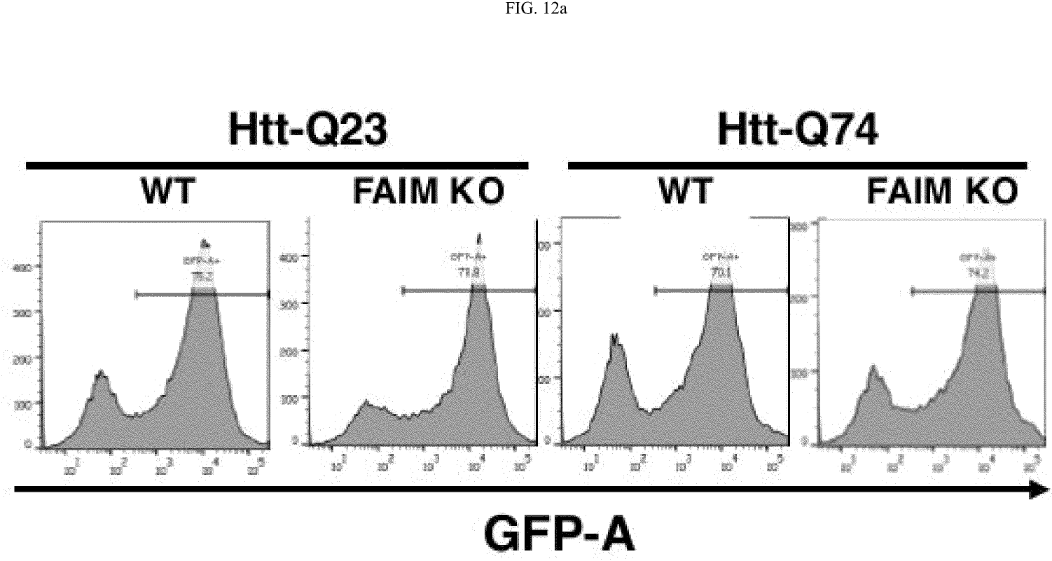

The associations among FAIM, ubiquitinated proteins and detergent insoluble material, induced by stress (FIG. 9a and FIG. 9b) suggest that impaired viability in stressed FAIM-deficient cells may be due to accumulation of cytotoxic, ubiquitinated protein aggregates. To determine if ubiquitinated protein aggregates increase after stress and do so disproportionately in the absence of FAIM, we assessed stress-induced accumulation of ubiquitinated proteins in FAIM KO HeLa cells vs WT HeLa cells by western blotting. We found ubiquitinated proteins accumulated in detergent-insoluble fractions after heat shock (FIG. 10a) and after oxidative stress (FIG. 10b) and did so to a much greater extent in FAIM KO HeLa cells compared to WT HeLa cells (FIGS. 10a and 10b).

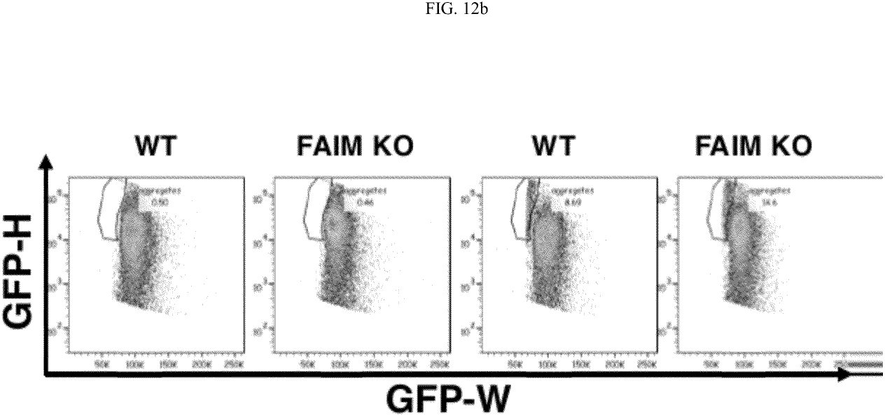

Next, to confirm that ubiquitinated proteins detected by western blotting in the detergent-insoluble fractions represent aggregated proteins, we performed filter trap assay (FTA) using total cell lysates from cells after oxidative stress. In this assay, large aggregated proteins are not able to pass though the 0.2 µm pore-sized filter and remain on the filter. We observed that more aggregated proteins from FAIM-deficient HeLa cell lysates (FIG. 10c) were trapped on the membrane during oxidative stress as compared to WT HeLa lysates. The same was true for primary mouse skin-derived fibroblasts from FAIM KO mice as compared to fibroblasts from WT mice (FIGS. 11a, b). Thus, following stress, FAIM directly binds ubiquitinated proteins that accumulate in detergent insoluble material, and accumulation of ubiquitinated proteins is much greater in the absence of FAIM. These results strongly suggest that FAIM is involved in the disposition of stress-induced aggregated proteins.

### Example 7 - Ubiquitinated Protein Aggregates Accumulate in FAIM-Deficient Tissues Following Oxidative Stress in Vivo.

To demonstrate that FAIM-deficiency correlates with more ubiquitinated protein upon cellular stress in vivo, we injected mice with menadione intraperitoneally, and assessed tissue injury. Liver and spleen were collected 18 hours after menadione administration into FAIM-deficient and littermate control FAIM-sufficient mice, and detergent-soluble and detergent-insoluble proteins were extracted. Similar to our in vitro experiments using HeLa cells and primary mouse fibroblasts, oxidative stress induced dramatically more ubiquitinated proteins in detergent-insoluble fractions from FAIM-deficient liver and spleen cells, as compared to liver and spleen cells from menadione-treated WT mice (FIG. 10d). In accordance with these results, we found much higher levels of menadione-induced serum LDH (FIG. 10e) and ALT (FIG. 10f), which are signs of cell injury and death, in FAIM KO as compared to WT mice. These data indicate that FAIM plays a non-redundant role in preventing accumulation of ubiquitinated, aggregated protein in stress-induced cells and animals, and in protecting against cell death.

### Example 8 - Aggregation-Prone Proteins Accumulate in FAIM-Deficient Cells Without Cellular Stress.

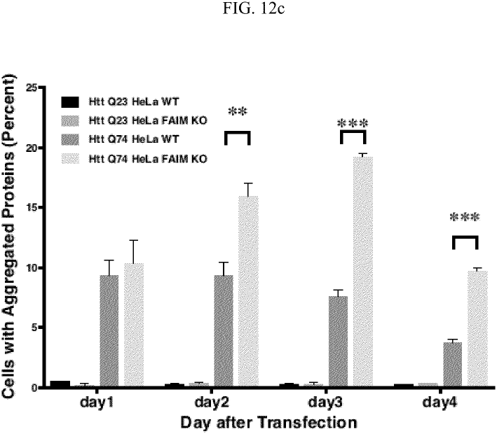

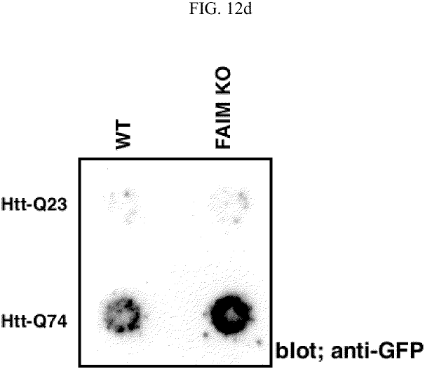

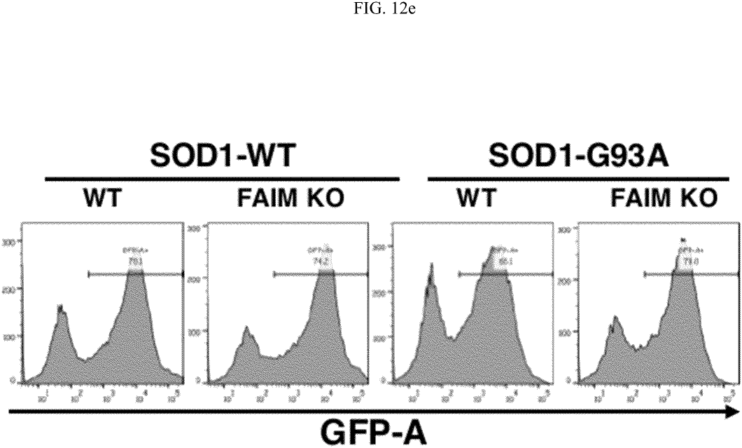

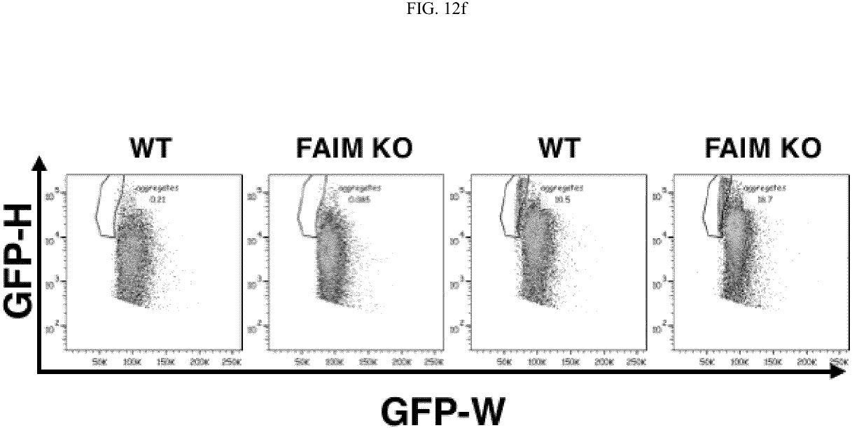

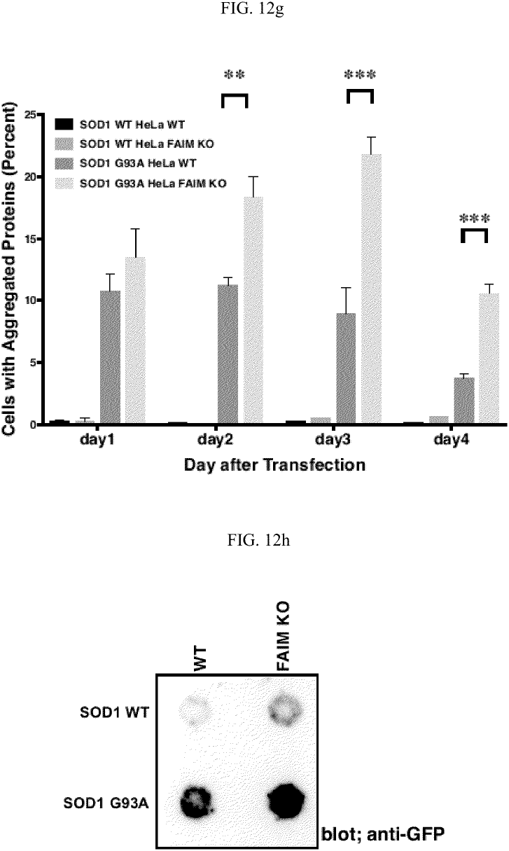

To directly assess the role FAIM plays in prevention of protein aggregation, we employed pulse shape analysis (PulSA) by flow cytometry to determine the level of aggregated protein. We transiently transfected HeLa cells with eGFP-tagged aggregation-prone proteins (mutant huntingtin exon1 and mutant SOD1 proteins) (FIGS. 12a, 12e), which spontaneously form aggregates in some cells. We found that cells containing aggregated proteins had a narrower and higher pulse shape of eGFP fluorescence than those that did not express protein aggregates, as previously reported (FIGS. 12b, 12f). Regardless of transfection efficiency (FIGS. 12a, 12e), the fraction of HeLa cells expressing aggregated proteins such as the huntingtin mutant (FIG. 12a) and the SOD1 mutant (FIG. 12e) was significantly higher in FAIM-deficient HeLa cells than in WT HeLa cells, especially at late stages after transfection (FIGS. 12c, 12g). These results were further verified by FTA. We found that much more aggregated mutant huntingtin (FIG. 12d) and aggregated mutant SOD1 (FIG. 12h) was filter trapped in FAIM-deficient HeLa cells than in WT HeLa cells. These results demonstrate the essential role of FAIM in altering the fate of mutant aggregation-prone proteins.

### Example 9 - Recombinant FAIM Inhibits Protein Fibrillization/Aggregation in an in Vitro Cell-Free System.

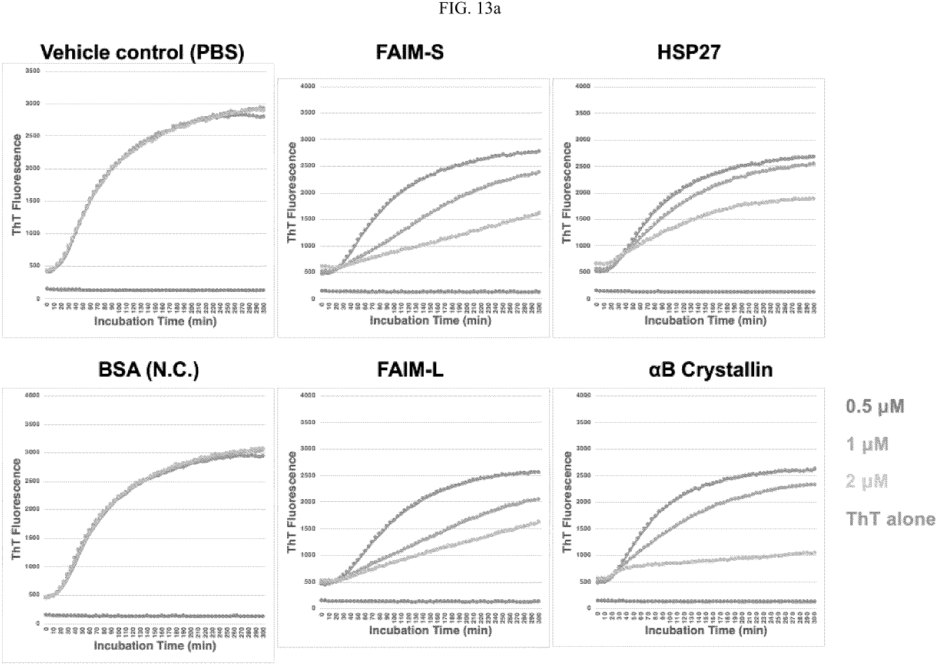

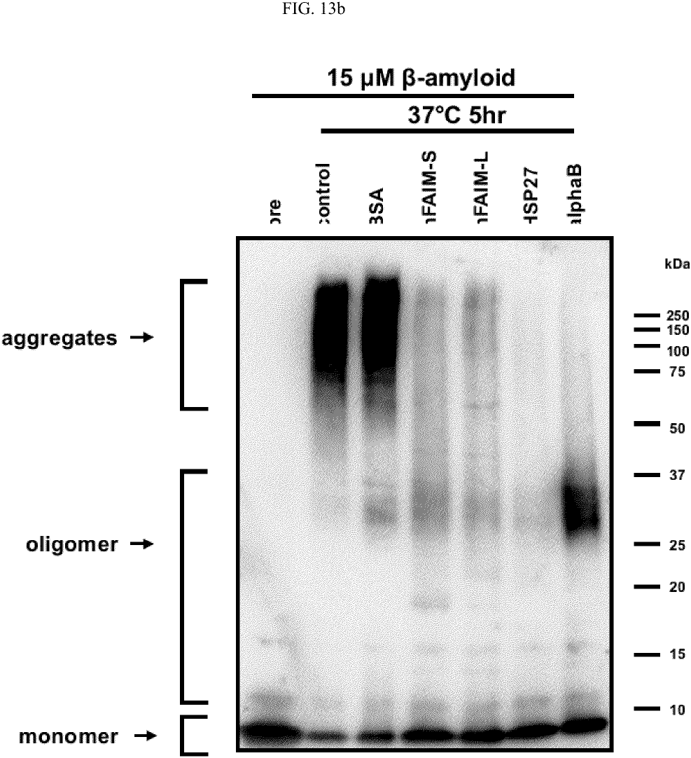

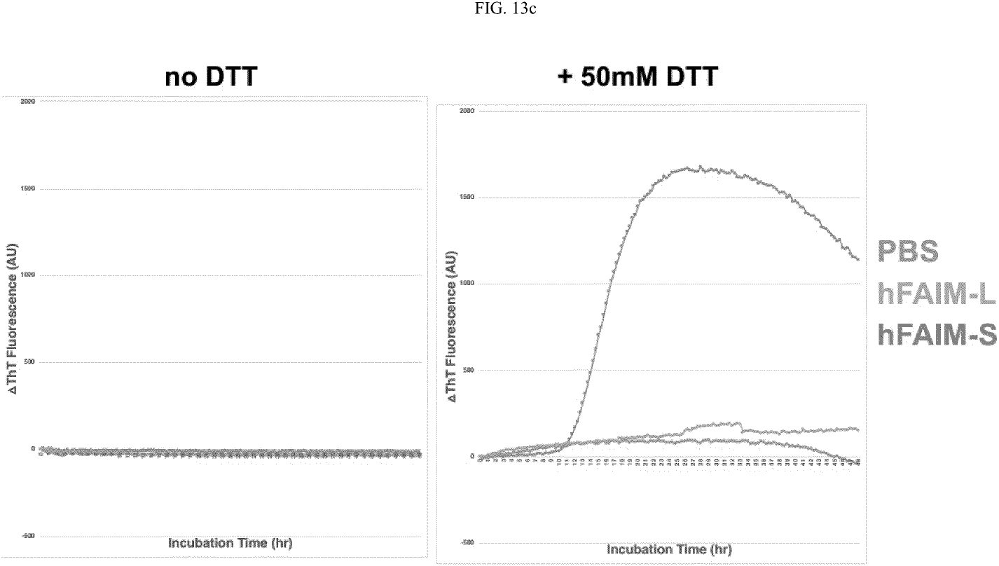

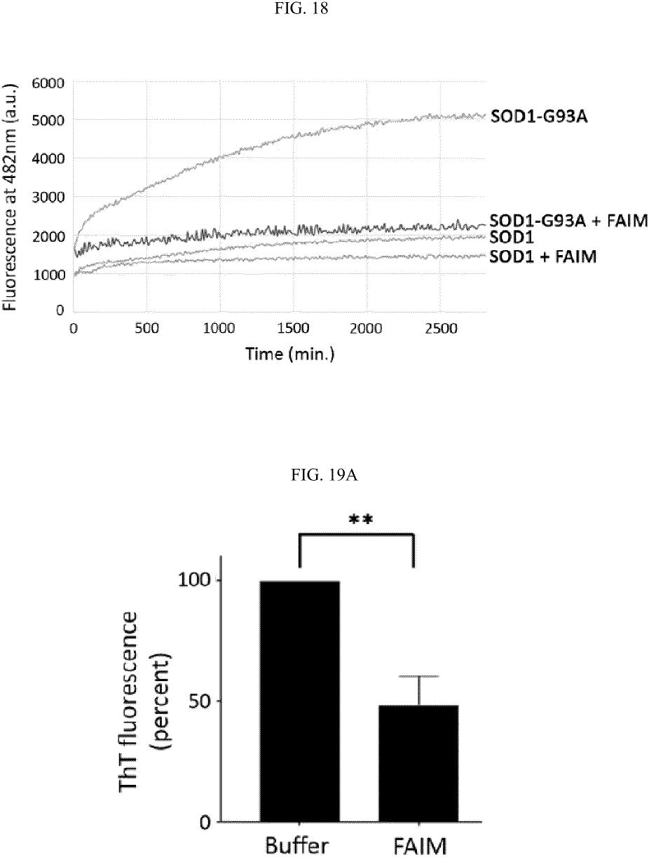

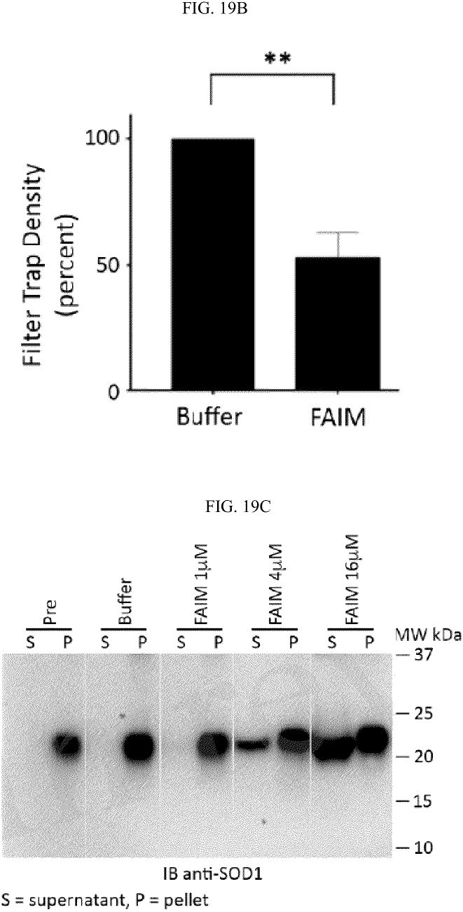

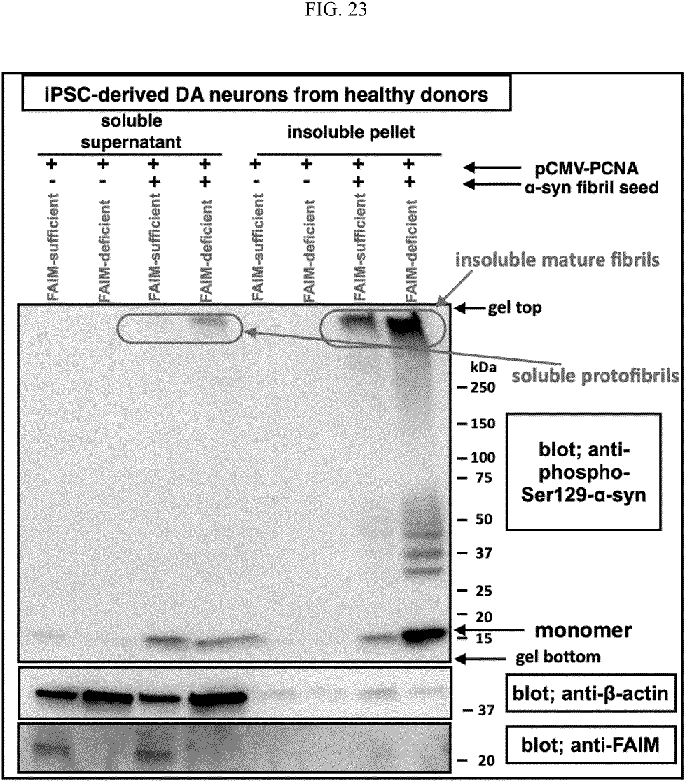

In order to examine whether FAIM directly inhibits protein aggregation, recombinant FAIM was mixed with aggregation-prone β-amyloid monomer (1-42) in an in vitro cell-free system and monitored aggregation status in real-time by ThT fluorescence intensity. sHSPs was also tested, because sHSPs are known to inhibit β-amyloid fibrillization/aggregation in cell-free systems and because HSP27 translocated to detergent-insoluble material in response to stress. It was found that β-amyloid aggregation was abrogated in the presence of recombinant FAIM or sHSPs in a dose-dependent manner (FIG. 13a). To confirm these results, aggregation status was assessed by western blotting SDS-PAGE, since aggregated proteins are SDS-resistant. It was observed aggregated β -amyloid in the high molecular weight range of negative controls (no added protein control, BSA control) and that the formation of high molecular weight aggregates was dramatically reduced in the presence of recombinant FAIM or sHSPs (FIG. 13b). In addition to β-amyloid, FAIM also inhibited DTT-induced aggregation of α-synuclein A53T mutant protein (FIG. 13c) and also inhibited aggregration of SOD1-G93A mutant protein (FIG. 18). The data using pure recombinant proteins in a cell-free system indicate that FAIM directly prevents protein fibrillization/aggregation. To evaluate whether FAIM c-terminal fragment (amino acid 90-179) is responsible for the prevention of β-amyloid aggregation, N-terminal-truncated FAIM-S were constructed. This mutant FAIM protein had similar ability to prevent β-amyloid aggregation. The data suggests that FAIM prevents protein aggregation via its c-terminal fragment (FIG. 22).

### Example 10A - Recombinant FAIM Reverses Protein Fibrillization/Aggregation in an in Vitro Cell-Free System.

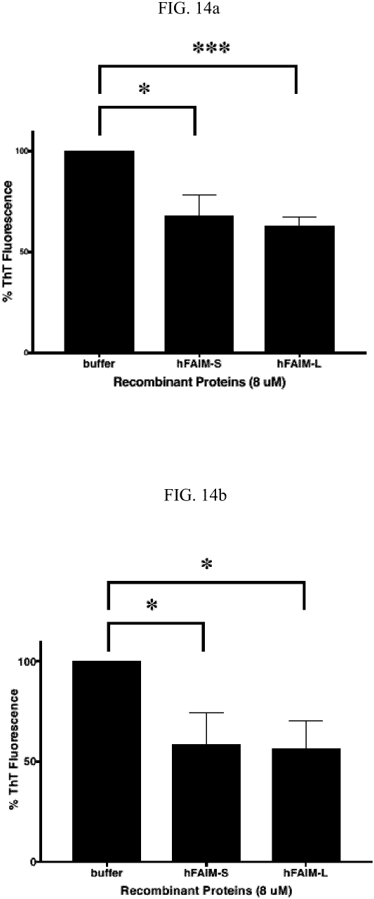

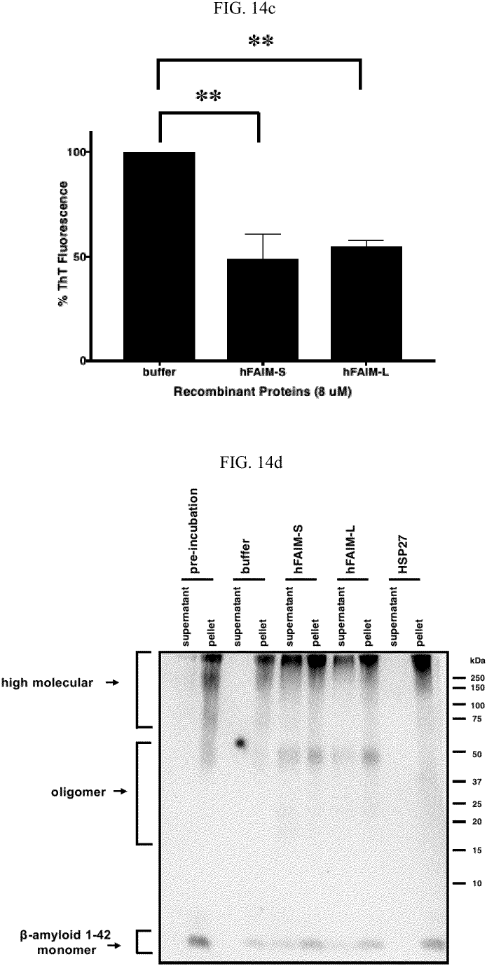

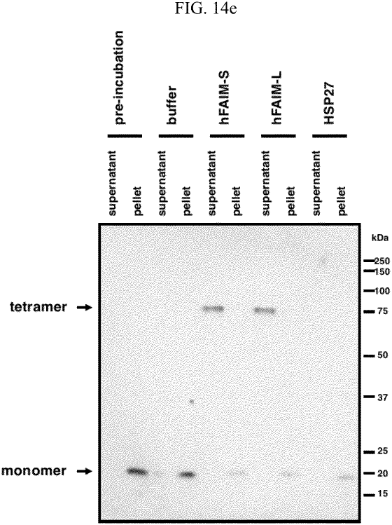

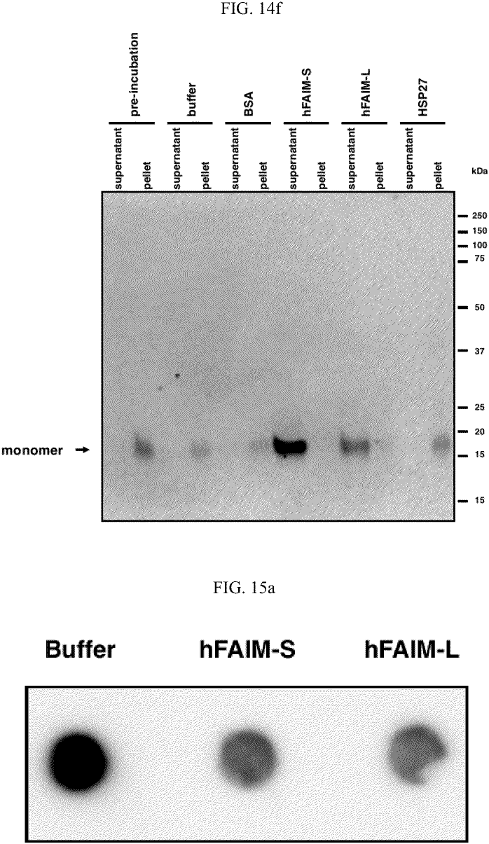

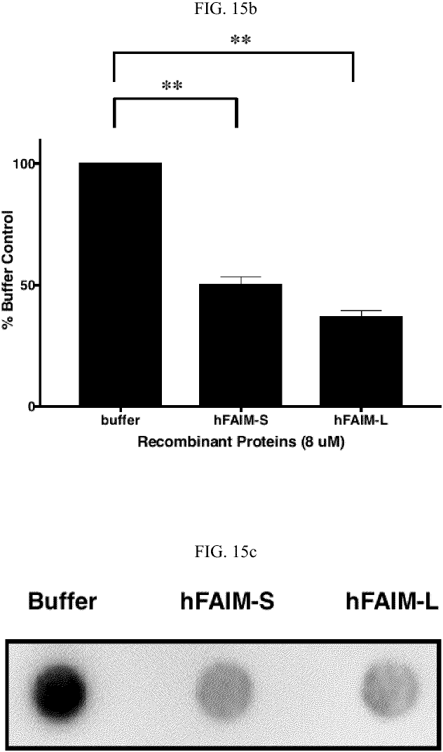

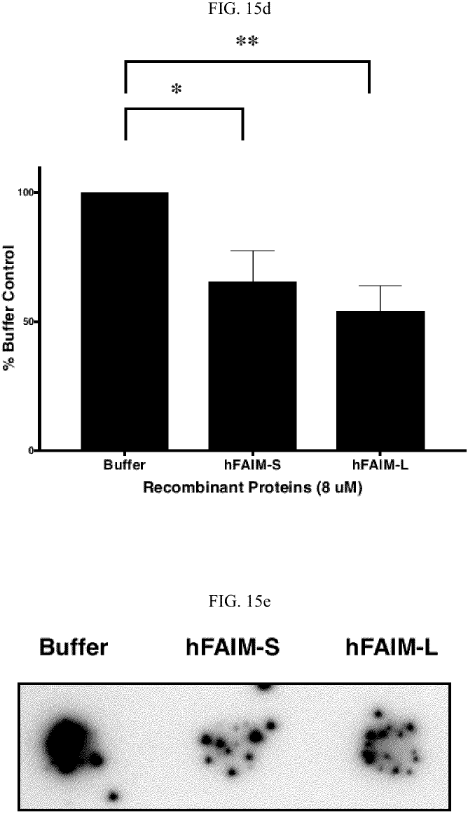

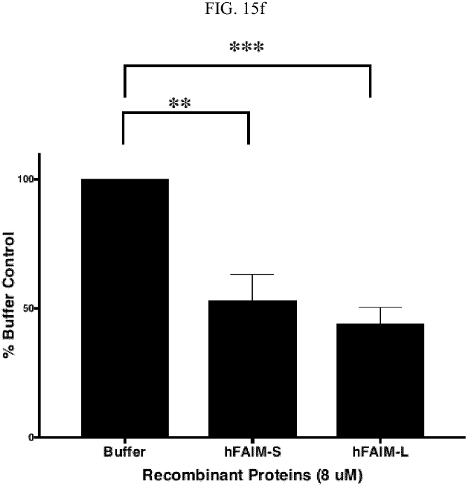

In order to examine whether FAIM is capable of reversing pre-formed, established protein aggregates in addition to preventing protein aggregation, we prepared β-amyloid aggregates and, after aggregate formation, added recombinant FAIM proteins. Aggregation status was monitored by ThT fluorescence and by FTA. We found that ThT fluorescence (FIG. 14a) and filter-trapped aggregates (FIGS. 15a, 15b) were significantly decreased after addition of FAIM as compared to negative controls. A similar pattern/phenomenon was observed using pre-aggregated α-synuclein A53T (FIG. 14b and FIGS. 15c, 15d) and pre-aggregated SOD1 G93A (FIG. 14c and FIGS. 15e, 15f).

We extended these results by examining protein aggregates with a complementary approach. We prepared β-amyloid aggregates and then added recombinant FAIM proteins, as before. We monitored aggregation status by differential sedimentation followed by solubilization in loading buffer and gel electrophoresis. Pre-formed β-amyloid aggregates alone appeared solely in the pellet fraction (FIG. 14d). We found that the addition of FAIM led to a shift in the bulk of previously aggregated β-amyloid proteins which now appeared as relatively high molecular mass species and oligomers in the supernatant fractions after SDS-PAGE (FIG. 14d). We found similar FAIM-mediated disassembly of α-synuclein A53T (FIG. 14e) and SOD1 G93A (FIG. 14f) preformed protein aggregates wherein disaggregated proteins translocated from pellet to supernatant fractions.

Unlike β-amyloid aggregates, α-synuclein A53T aggregates disassembled by FAIM and located in supernatant fractions appeared as tetramers on SDS-PAGE, as previously reported, rather than monomers (FIG. 14e). Along the same lines, SOD1 G93A presenting as detergent insoluble aggregates, as well as FAIM-disassembled supernatant material, was fully dissolved by loading buffer and appeared as monomers on SDS-PAGE (FIG. 14f). These results indicate that, in an in vitro cell-free system, established protein aggregates of β-amyloid, α-synuclein, and SOD1 can be dissolved by FAIM, at least in part.

### Example 10B - FAIM Opposes Tau in the Brain

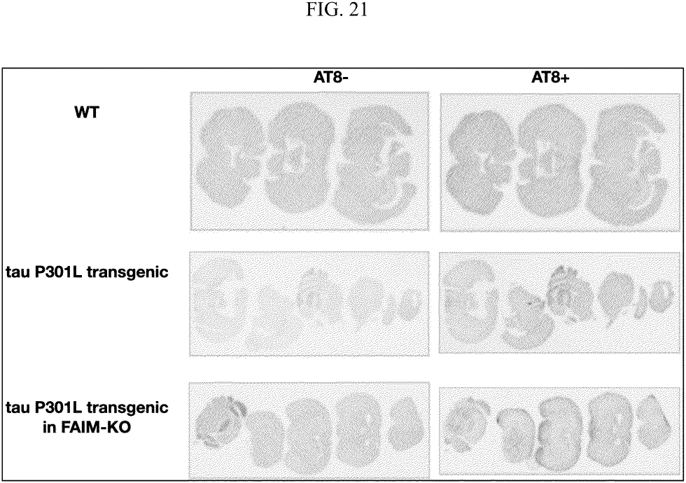

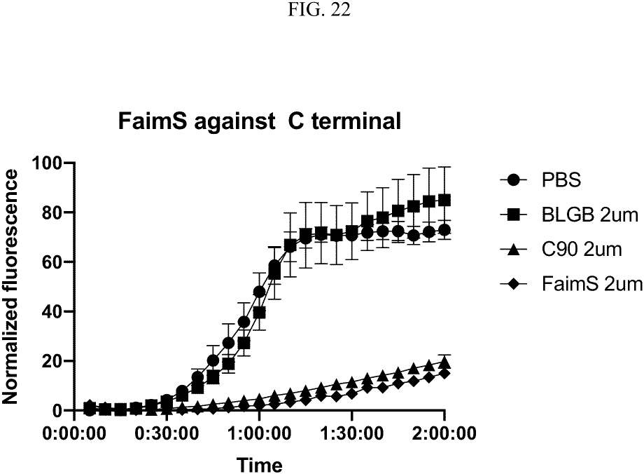

To extend our findings on the role of FAIM in opposing dysfunctional protein aggregation to an in vivo system, we crossed FAIM KO (FAIM-deficient) mice with tau P301 transgenicc mice that are a model for Alzheimer’s Disease. We analyzed brains from these mice by immunohistochemistry with antibody AT8 (mouse anti-phosphotau monoclonal antibody) and found increased phosphorylated tau levels in the frontal cortex and hypothalamus regions of the FAIM KO (FAIM-deficient) mice as compared to wild-type (normal) mice at 12 month of age (FIG. 21).

### Example 11 - Analysis and Discussion of FAIM

Without wishing to be bound by any particular theory, the foregoing examples are discussed. Although the FAIM gene arose in the genomes of the last common holozoan ancestor with a high level of homology among holozoan species, similar to house-keeping genes, its physiological function has been a long-standing enigma. Here, we have demonstrated that FAIM, originally thought of as a FAS-apoptosis inhibitor, plays an unexpected, non-redundant role in protection from cellular stress and tissue damage, leading to improved cellular viability. We have elucidated FAIM’s molecular mechanism and demonstrated that it directly interacts with ubiquitinated protein aggregates, rather than opposing caspase activity or dampening ROS generation. We have further documented the capacity of FAIM to prevent protein fibrillization/aggregation and to dissociate pre-formed protein aggregates, strongly suggesting that FAIM play a distinctive, non-redundant, HSP104-like role in advanced organisms, for which no other presently known metazoan protein can fully substitute.

Cells and tissues are continuously subjected to environmental insults such as heat shock and oxidative stress, which cause accumulation of cytotoxic, aggregated proteins. Organisms have evolved protective cellular mechanisms such as HSPs in order to prevent and counteract tissue and organ damage. Our data supports the role of FAIM as a new player that not only antagonizes protein aggregate formation but uniquely functions to disassemble established aggregates.

Additional evidence supporting similar functions of FAIM and sHSPs was obtained from overexpression experiments. First, NGF-induced neurite outgrowth in vitro was promoted by overexpression of FAIM in the PC12 cell line and by overexpression of HSP27 in dorsal root ganglion neurons. Second, FAS-mediated apoptosis was inhibited by overexpression of FAIM in B lymphocytes, in PC12 cells and in HEK293T cells and cortical neurons, and by overexpression of HSP27 in L929 cells. Finally, NF-κB activation was enhanced by overexpression of FAIM in NGF-treated PC12 cells and in CD40-stimulated B lymphocytes whereas overexpression of HSP27 enhanced NF-κB activation in TNFα-treated U937 cells and MEF cells. It is possible some of these FAIM effects could be artifacts of overexpression due to protein/gene dosage imbalances that alter biological outcomes, rather than from direct biological effects of FAIM or HSP27, or, alternatively, could be due to FAIM- or HSP27-mediated maintenance of cell viability. However, despite these many similarities between FAIM and heat shock proteins, FAIM is not a sHSP. FAIM is not homologous with sHSPs and contains no α-crystallin domain, and, most importantly, the function of FAIM goes beyond preventing aggregation of damaged proteins to disassembling pre-formed, established protein aggregates, something that HSPs are incapable of doing.

Thus, the results presented herein suggests that there are 3, rather than 2, potential fates for stress-induced, disordered proteins and their aggregates. They may be ubiquitinated and disposed via the proteasome system, or, they may be ubiquitinated and eliminated via autophagy; however, particularly in situations in which the accumulation of disordered proteins exceeds the capacity of proteasome/autophagy handling and aggregation ensues, they may be disassembled, disaggregated and solubilized by FAIM.

The aggregation of proteins into fibrillar high molecular-weight species is a hallmark of numerous human neurodegenerative disorders. In the situation where overwhelming generation of misfolded or aggregated proteins due to cellular or aging stress occurs, these cytotoxic species must be degraded. However, in normal aged neuronal cells, autophagy-related genes are downregulated, leading to dysfunction of autophagy-mediated aggregate clearance. Interestingly, autophagy was found to be impaired in Huntington’s disease model mice and patients. Further, proteasomal function has been reported to decline with age. Thus, in a situation of low autophagic and proteasomal activity, the role of FAIM in preventing aggregation, and/or reversing aggregation, may be crucial to maintain proteostasis.

The pathophysiology of the neurodegenerative disorder, AD, involves protein aggregates in the form of β-amyloid plaques and tau neurofibrillary tangles. An association between AD and FAIM has been suggested. FAIM-L expression was found to be impaired in the brains of AD patients, especially in the late BRAAK stages. Given that FAIM protein prevented and reversed β-amyloid aggregation in vitro, we suggest the novel hypothesis that low/no FAIM expression might be pathogenically linked to more rapid, aggressive, overwhelming β-amyloid aggregation in AD patients rather than a marker of AD progression.

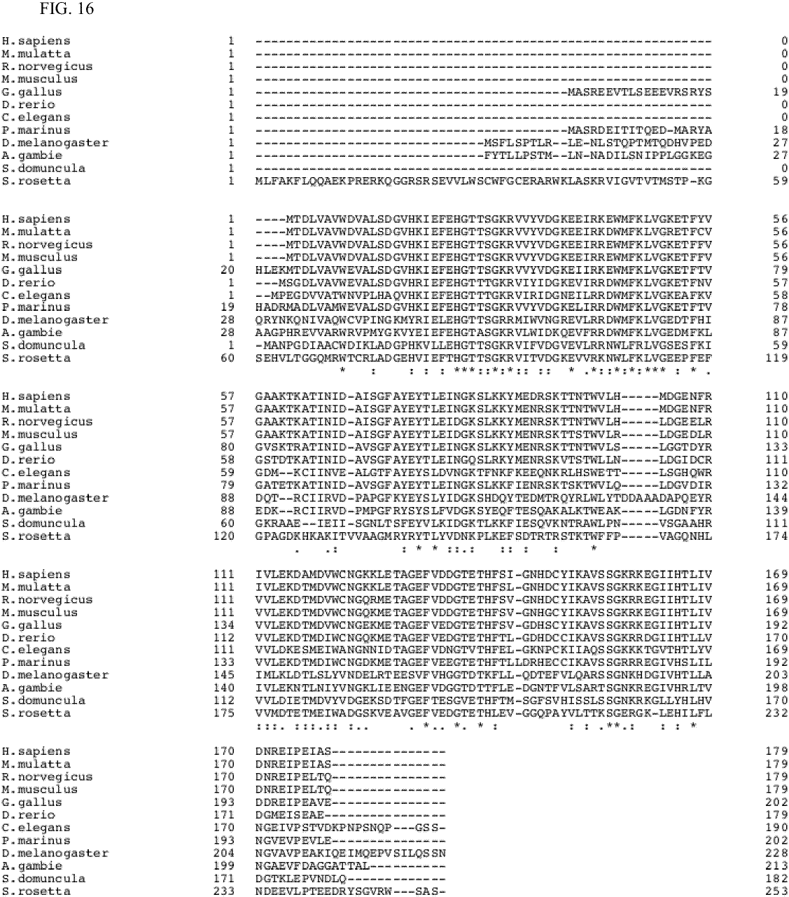

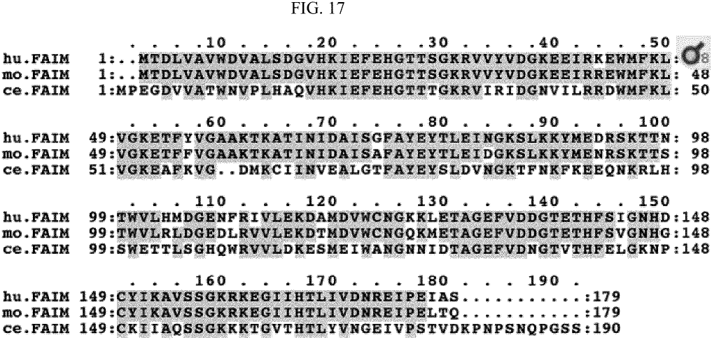

Prior to our discovery that FAIM can dissociate aggregated proteins, HSP104 had been previously shown to have this unique function. HSP104 and its homologs exist only in the genomes of plants, bacteria, yeast and choanoflagellates, but interestingly, is absent from metazoan organisms. In contrast, FAIM arose in the genomes of choanoflagellates and has evolved throughout holozoan species (FIG. 16 and FIG. 17). ATP is required for disaggregation activity by HSP104 whereas our work demonstrates that FAIM disaggregates proteins in the absence of ATP. In fact, there is no ATP-binding site in the FAIM protein. One can envisage that FAIM might have replaced the function of HSP104 in metazoan species to spare ATP for active movement in order to increase the survival rate of multicellular organisms.

Recently it has been suggested that a tripartite complex of HSP70 combined with HSP40 (J protein) and HSP110 is capable of dissociating aggregated proteins. As with HSP104, this occurs in an ATP-dependent manner. Thus, FAIM is unique in being a single, ATP-independent protein that dissociates aggregated proteins, and is the only such metazoan protein with these characteristics known at this time. From the clinical-translational standpoint, manipulation of a single FAIM protein that does not require ATP for function is likely to be more feasible than manipulation of a multimember protein complex.

It has been suggested that solubilization of β-amyloid aggregates and/or tau aggregates is the first necessary step to treating AD with antibodies that can then hasten disposal. Although Hsp104, which is lost from metazoa, can disaggregate proteins and could be a candidate for disaggregation therapy for neurodegenerative diseases, it might cause neuroinflammation because HSP104 is a foreign antigen, which could elicit potent, unwanted immune responses. In contrast, FAIM is highly evolutionarily conserved and is a natural protein product to humans making it an attractive target for therapeutic intervention. Taken together, our work provides new insights into the interrelationships among FAIM, protein aggregation and cell viability that may have applicability to neurodegenerative diseases, which could potentially lead to species-compatible, rationally designed preventive and therapeutic interventions.

### Example 12 - FAIM and SOD1 Methods and Reagents

The following methods were used in Examples 13 to 16.

### Cell Culture and Transfection

HeLa cells were obtained from the American Type Culture Collection (ATCC). HeLa cells were cultured in DMEM medium (Corning®) containing 10% FCS, 10 mM HEPES, pH 7.2, 2 mM L-glutamine and 0.1 mg/ml penicillin and streptomycin. Transfection was performed using Lipofectamine 3000, according to the manufacturer’s instructions (Invitrogen®).

### Generation of FAIM Knockout Cells With CRISPR/Cas9

Guide RNA (gRNA) sequences for the human FAIM gene (FIG. 20) were designed using a CRISPR target design tool (http://crispr.mit.edu) in order to target the exon after the start codon. Annealed double strand DNAs were ligated into pSpCas9(BB)-2A-GFP (PX458) vector (Addgene) at the Bpi1 (Bbs1) restriction enzyme sites using the ‘Golden Gate’ cloning strategy. The presence of insert was verified by sequencing.

Empty vector was used as a negative control. Transfection was performed using lipofection and a week after transfection, eGFP⁺ cells were sorted with an Influx instrument (Becton Dickinson), and seeded into 96 well plates. FAIM knockout clones were screened by limiting dilution and western blotting.

CRISPR-Cas9 oligonucleotides used for this work were as follows: human FAIM forward, “

CACCGACAGATCTCGTAGCTGTTTGGG (SEQ ID NO: 41)

”; human FAIM reverse, “

AAACAAACAGCTACGAGATCTGTC (SEQ ID NO: 42)

.”

### Plasmids

The following plasmids were obtained from Addgene®.
pF146 pSOD1WTAcGFP1, #26407
pF150 pSOD1G93AAcGFP1, #26411
pSpCas9(BB)-2A-GFP (PX458), #48138
Pulse-shape analysis (PulSA)

WT and FAIM KO HeLa cells were transiently transfected with an eGFP-tagged native human SOD1 or aggregation-prone SOD1-G93A protein expression vector. Cells expressing eGFP-tagged SOD1-G93A protein were harvested at the indicated times and eGFP expression was analyzed on an LSR Fortessa (BD Pharmingen®) flow cytometer for PulSA analysis to detect protein aggregates. Data was collected in pulse-area, height and width for each channel. At least 10,000 cells were analyzed.

### Filter Trap Assay (FTA)

WT and FAIM KO HeLa cells were transiently transfected with an eGFP-tagged native human SOD1 or aggregation-prone human SOD1-G93A protein expression vector, and fluorescently tagged cells were then harvested at 48 hours. Cells were washed with PBS and then lysed in PBS containing 2% SDS, 1 mM MgCl2, protease inhibitor cocktail and 25 unit/ml Benzonase (Merck®). Protein concentrations were quantified using 660 nm Protein Assay Reagent with Ionic Detergent Compatibility Reagent (IDCR) (ThermoFisher Scientific®). Equal amounts of protein extracts underwent vacuum filtration through a 0.2 µm pore size cellulose acetate membrane (GE Healthcare®) for the detection of SOD1 aggregates using a 96 well format Dot-Blot apparatus (Bio-Rad®). The membrane was washed twice with 0.1% SDS in PBS and western blotted using anti-GFP antibody (Cell Signaling Technology®) to detect aggregated proteins. Cell free aggregates of SOD1-G93A were similarly applied to nitrocellulose membrane. In cell-free experiments, SOD 1 protein was vacuum filtered as above, after which membranes were western blotted using anti-SOD1 antibody (Cell Signaling Technology®) to detected aggregated proteins.

### Thioflavin T Fluorescence Assay

A Fibril/aggregate formation of mutant SOD1-G93A (10 µM) was assessed in the presence or absence of FAIM (4 µM) by Thioflavin T (ThT, 20 µM) (Sigma-Aldrich®) fluorescence using a Synergy Neo2 Multi-Mode Microplate Reader (Bio-Tek). Reader temperature was set at 37° C. with continuous double orbital shaking at a frequency of 425 cpm at 3 mm between reads. Aggregation conditions required the presence of the reducing agent TCEP (tris(2-carboxyethyl)phosphine) (Sigma-Aldrich®) at 20 mM and EDTA at 5 mM, in the presence of an extreme-temperature slippery PTFE Teflon® beads (McMaster-Carr). ThT fluorescence intensity was measured using an excitation wavelength of 440 nm and an emission of 482 nm. Photomultiplier (PMT) gain was set at 75. Fluorescence measurements were made from the top of the plate, with the top being sealed with an adhesive plate sealer to prevent evaporation.

### Disaggregation Assays

SOD1-G93A (2 µM) pre-formed fibrils were incubated with 8 µM recombinant FAIM at 37° C. for 2.5 hours. Then, fibril status was determined by either ThT fluorescence, FTA, or by detecting proteins in the supernatants and in the pellets after sedimentation at 21,000xg and western blotting.

### Western Blotting

Protein concentrations were determined using the 660 nm Protein Assay Reagent (Pierce). Protein samples in 1 x Laemmli buffer with 2-mercaptoethanol at 2.5% were boiled for 5 min. Equal amounts of protein for each condition were subjected to SDS-PAGE on an AnykD gradient gel (Bio-Rad®) followed by immunoblotting with anti-SOD1 antibody (Cell Signaling®) after wet transfer for one hour to PVDF membrane (Bio-Rad) and blocking with nonfat dry milk.

### His-Tag Recombinant Protein Production

His-tag protein expression vectors were constructed using pTrcHis TA vector according to the manufacturer’s instructions. In brief, PCR amplified target genes were TA-cloned into the vector (Invitrogen®) and inserted DNA was verified by sequencing (Genewiz®). Proteins were expressed in TOP10 competent cells (Invitrogen) with IPTG at 1 mM and were purified using a Nuvia IMAC Nickel-charged column (Bio-Rad) on an NGC Quest chromatography system (Bio-Rad®). Protein purity was verified using TGX Stain-Free gels (Bio-Rad) on ChemiDoc Touch Imaging System (Bio-Rad®) and each protein was determined to be >90% pure.

After elution from a nickel-charged column, aggregation-prone SOD1 was generated by demetallization with EDTA under reducing conditions. Purification was performed in the presence of guanidine HCl to induce dimer subunit disassociation, followed by three stage dialysis buffer exchange in the presence of EDTA at 5 mM to remove metal ions.

Oligonucleotides used in this work for cloning into pTrcHis TA vector were as follows: human FAIM forward, “

ATGACAGATCTCGTAGCTGTTTGG

” (SEQ ID NO: 62); human FAIM reverse, “

TTAACTTGCAATCTCTGGGATTTC

” (SEQ ID NO: 63); human SOD1 forward, “

ATGGCGACGAAGGCCGTGTG

” (SEQ ID NO: 64); human SOD1 reverse, “

TTATTGGGCGATCCCAATTACAC (SEQ ID NO: 38)

.” Sequence primers used in this work were as follows: for pX-458, “

TGGACTATCATATGCTTACCGTAACTTGAAAG

” (SEQ ID NO: 65); for pTrcHis TA, “

TATGGCTAGCATGACTGGT (SEQ ID NO: 66)

.”

Work described herein was carried out with adherence to all institutional safety procedures.

### Generation of Pre-Formed Protein Aggregates for Disaggregation Assays

SOD1-G93A fibrils were assembled in an Eppendorf ThemoMixer F1.5 with ThermoTop, as previously described (25), with minor modifications. SOD1-G93A (80 µM) fibrils were generated in assembly buffer (AB; 40 mM HEPES-KOH pH 7.4, 150 mM KCl, 20 mM MgCl2 and 1 mM dithiothreitol) plus 10% (v/v) glycerol for 16 hours with agitation. Fibrils were recovered by centrifugation, washed and resuspended in assembly buffer for disaggregation assays. For all fibrils, generation was confirmed by ThT fluorescence. Fibrils were diluted to the requisite concentration for subsequent disaggregation reactions (FIG. 14f). Statistics

All quantitative data are expressed as mean ± SEM. ANOVA or, when appropriate, unpaired t-test was used for statistical determinations with GraphPad Prism 7 software. Values of p<0.05 are considered statistically significant (*p<0.05, **p<0.01 or ***p<0.001).

### Example 13 - Activity of FAIM With Respect to the Disease-Associated, Aggregation-Prone Mutant Protein, SOD1 and α-Syn, and Tau.

To examine the activity of FAIM with respect to the disease-associated, aggregation-prone mutant protein, SOD1, we first deleted FAIM from HeLa cells by CRISPR/Cas9 excision. We then transiently transfected FAIM-deficient HeLa cells and WT HeLa cells with eGFP-tagged mutant SOD1-G93A, which spontaneously forms aggregates. To directly assess the role FAIM plays in prevention of SOD1-G93A protein aggregation, we employed two assays: First, we used pulse shape analysis (PulSA) by flow cytometry to determine the level of aggregated protein. In this assay, cells containing aggregated proteins have a narrower and higher pulse shape of eGFP fluorescence than those that do not express protein aggregates. We found that regardless of transfection efficiency, the fraction of HeLa cells expressing aggregated mutant SOD1 was significantly higher in FAIM-deficient HeLa cells than in WT HeLa cells, especially at late stages after transfection (FIGS. 12e-f); second, we used filter trap assay (FTA) to evaluate the level of aggregated protein. In this assay, large aggregated proteins are not able to pass through a 0.2 µm pore-sized filter, remain on the filter, and are blotted with anti-GFP antibody. We found that much more aggregated mutant SOD1 was filter trapped in FAIM-deficient HeLa cells than in WT HeLa cells (FIG. 12h). Using a cellular α-syn seeding model, it was found similar accumulation of misfolded α-syn species in FAIM-deficient iPSC-derived dopaminergic neurons from healthy donors as judged by western blot (WB) using an anti-phospho-serine 129 α-syn antibody because phosphorylation at serine129 (pS129) of α-syn plays an important role in the regulation of α-syn oligomerization/fibrillization, Lewy body (LB) formation, and neurotoxicity (FIG. 23). These results demonstrate the essential role of FAIM in blocking formation of mutant SOD1 and α-syn aggregates.

### Example 14 - Indirect And/or Direct Activity of FAIM in Opposing Mutant SOD1 Aggregation in Cells.

The activity of FAIM in opposing mutant SOD1 aggregation in cells could be direct or indirect, the latter potentially involving other cellular elements. In order to address this issue, we established a cell free assay for evaluating FAIM function by testing the ability of FAIM to interfere with generation of mutant, ALS-associated SOD1 aggregates. We examined recombinant SOD1-G93A alone, and with FAIM, and monitored the onset of aggregation by Thioflavin T (ThT) fluorescence (excitation at 440 nm and emission at 482 nm). ThT fluorescence increases with increasing aggregation and fibril formation. We also tested native SOD1 alone and with FAIM. As shown in FIG. 18, aggregates of SOD1-G93A formed over a 48 hour time course as detected by increasing ThT fluorescence and this was largely prevented by the presence of FAIM. In contrast, native SOD1 did not aggregate and fluorescence for native SOD1 was little affected by FAIM. Thus, acting alone, FAIM is capable of interfering with the formation of mutant SOD1 aggregation.

### Example 15 - Ability of FAIM to Act on Established Aggregates

We then examined the possibility that FAIM can act on established aggregates. To test this, we generated recombinant mutant SOD1-G93A protein aggregates in a cell free system as described in Methods (in the section on generation of pre-formed protein aggregates for disaggregation assays). We subsequently added recombinant FAIM, and monitored the level of aggregation by ThT fluorescence and by filter trap assay. No other reagents or additives (including no ATP) beyond buffer were present. We found marked reduction of protein aggregation in both assays in terms of reduced ThT fluorescence (FIG. 14c) and reduced filter trapped (FTA) protein (FIG. 15e), 2.5 hours after addition of 8 µM FAIM, as compared to no FAIM addition (“Buffer”). These findings demonstrate the activity of FAIM in disassembling mutant SOD1 aggregates.

We further evaluated the disaggregating activity of FAIM through differential sedimentation. In this approach, SOD1-G93A protein aggregates are pelleted during high speed centrifugation (21,000xg), leaving only low molecular size species or monomers in the supernatant. Mutant SOD 1 in both the pellet and supernatant fractions is subsequently solubilized to monomers by SDS-PAGE prior to western blotting. As expected, pre-formed SOD1-G93A aggregates (PRE) appeared solely in the pellet fraction (P) and were solubilized in loading buffer (FIG. 14f). However, we found that addition of FAIM led to a dose-dependent shift in previously aggregated mutant SOD1, which now appeared in the supernatant fraction (S), indicating dissolution to lower molecular (disaggregated) forms (FIG. 14f). Addition of buffer had no effect. In other words, FAIM treatment leads to a physical shift of preformed SOD1-G93A aggregates from the sedimented pellet fraction to the non-sedimentable, soluble, supernatant fraction. In total, results from ThT, FTA and sedimentation indicate that FAIM, in the absence of any other factors, can disassemble/disaggregate protein aggregates composed of mutant SOD1.

### Example 16 - Analysis and Discussion of FAIM

FAIM was originally cloned as a molecule that inhibits Fas death receptor induced apoptosis in mouse B lymphocytes. The FAIM sequence is unique, and is not related in either its short or long form of 179 or 201 amino acids, respectively, to two other gene products confusingly termed FAIM2 and FAIM3 by other groups. The true function of FAIM protein has been unknown for many years. In retrospect, its role was likely obscured by the lack of stress in vivarium mouse life. Recently, we showed that FAIM uniquely and non-redundantly opposes stress-induced cell death and stress-induced accumulation of protein aggregates in multiple cell types in vitro and in mice in vivo. Here we show that FAIM plays a key role in cellular proteostasis, and specifically acts to prevent mutant SOD1 aggregation (in cell lines and in vitro) and to reverse established mutant SOD1 aggregates (in vitro). Thus FAIM is capable of interfering with SOD1-G93A aggregation and disassembling SOD1-G93A aggregates without the need for other cellular or soluble elements, including without the need for ATP. The data also shows that FAIM similarly acts to prevent aggregation of mutant α-synuclein and β-amyloid and to reverse established aggregates of mutant α-synuclein and β-amyloid. We hypothesize that FAIM can play a role in preventing and/or reversing dysfunctional mutant SOD1 protein aggregation that is generally acknowledged as being responsible, all or in part, for some cases of clinical FALS disease. However, other SOD1 mutations, beyond G93A, have been implicated in the pathogenesis of FALS, as have mutations in other proteins, and it is important to point out that at the present time the activity of FAIM beyond SOD1-G93A has not been defined for other SOD1 mutations. However, the activity of FAIM in preventing and reversing aggregation of α-synuclein and β-amyloid that are generally acknowledged as being responsible, all or in part, for some cases of PD and AD, suggests FAIM can play a role in preventing and/or reversing PD and AD, as well as FALS.

Other proteins have been shown to affect protein aggregates. Much work has been carried out with a disaggregating protein from yeast, HSP104, including modification to broaden and enhance its activity. But there is no vertebrate, let alone mammalian, homolog of HSP104. Although this work clearly demonstrates proof of concept, HSP104 is a foreign protein for humans and so is unlikely to represent a feasible treatment because of the expected human anti-yeast response. Further, HSP104 requires ATP for function and it is unclear whether this would pose a functional limitation. The multiprotein combination of mammalian HSP110/70/40 opposes protein aggregation, but the need for 3 different proteins is likely to limit therapeutic utility. Like HSP104, HSP110/70/40 also requires ATP for optimal activity. Nicotinamide mononucleotide adenylyl transferase (NMNAT) in conjunction with HSP90, and the PDZ serine protease HtrA1, have been reported to disassemble some protein aggregates under limited circumstances. Other proteins, including HSP27 and αBcrystallin, have been shown to interfere with protein aggregate formation, including formation of SOD1-G93A aggregates, but neither appears capable of disaggregating established aggregates. Thus, at this point in time, and to the best of our knowledge, FAIM is the only mammalian protein that works alone, without the need for ATP, to both prevent and reverse aggregation of mutant SOD1. As such there is reason to evaluate FAIM activity with respect to other aggregation-prone proteins and to determine whether FAIM has any effect on the course of ALS-like disease or other diseases in which protein aggregation is implicated in pathogenesis.

The true role of this highly conserved protein has been obscure until now because of the lack of known consensus effector/binding motifs, the lack of even partial sequence homology with any other protein, plus our finding that mice lacking FAIM evidence no obvious abnormality and experience healthy lives and normal lifespans within the confines of our specific pathogen-free animal colony. Two results indicate that FAIM adopts a beta pleated sheet clamshell like structure. An NMR study indicated this kind of structure is present in the C-terminal domain whereas the N-terminal domain is relatively unstructured. X-ray crystallographic data indicates that both the C-terminal and N-terminal domains are arranged as clamshells. Inasmuch as SOD1 contains a beta barrel, these studies lead to speculation that the FAIM and SOD1 beta structured regions may intercalate which might interfere with, or disrupt, aggregation. More discrete, structure-function study is likely to reveal one or more novel motifs important for opposing protein aggregation and promoting proteostasis, which may involve beta structure or other structural elements.

There are a limited number of mechanisms for addressing dysfunctional and disordered proteins. These include degradation via the proteasomal system and disposal via the autophagic pathway, along with renaturation mediated by heat shock proteins (HSPs). The failure of any of these systems to compensate for the loss of FAIM indicates that FAIM activity represents a separate, distinct, and independent pathway for dealing with proteins that are born, or made, atypical and aggregate. The very long evolutionary history of FAIM suggests the possibility that it is first among mammalian proteins that developed to counteract protein aggregation, eliminate aberrant proteins, and maintain proteostasis, and still manifests unique, non-complementary activity (FIG. 16).

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or fragments thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A peptide or mimetic thereof comprising an amino acid sequence having at least 70% sequence identity to
MEDRSKTTNTWVLHMDGENFRIVLEKDTMDVWCNGKKLETAGEFVDDGTE
THFSIGNHDCYIKAVSSGKRKEGIIHTLIVDNREIPEIAS (SEQ ID N
O: 6)
.

2. The peptide of claim 1, wherein the amino acid sequence has at least 90% sequence identity to SEQ ID NO: 6.

3. The peptide of claim 1, wherein the amino acid sequence has at least 95% sequence identity to SEQ ID NO: 6.

4. The peptide of claim 1, wherein the peptide exhibits ability to disaggregate protein complexes.

5. A peptide or mimetic thereof comprising an amino acid sequence having at least 70% sequence identity to
MEDRSKTTNTW (SEQ ID NO: 7)
,
VLHMDGENFR (SEQ ID NO: 8)
,
IVLEKDTMDV (SEQ ID NO: 9)
,
WCNGKKLETA (SEQ ID NO: 10)
,
GEFVDDGTET (SEQ ID NO: 11)
,
HFSIGNHDCY (SEQ ID NO: 12)
,
IKAVSSGKRK (SEQ ID NO: 13)
,
EGIIHTLIVD (SEQ ID NO: 14)
, or
NREIPEIAS (SEQ ID NO: 15)
, wherein the peptide has a length of at least 10 amino acid residues.

6. The peptide of claim 5, wherein the amino acid sequence has at least 90% sequence identity to SEQ ID NO: 7, 8, 9, 10, 11, 12, 13, 14, or 15.

7. The peptide of claim 5, wherein the amino acid sequence has at least 95% sequence identity to SEQ ID NO: 7, 8, 9, 10, 11, 12, 13, 14, or 15.

8. The peptide of claim 5, wherein the peptide has a length of at least 15 amino acid residues.

9. The peptide of claim 5, wherein the peptide exhibits ability to disaggregate protein complexes.

10. A composition comprising:
a peptide or mimetic thereof comprising amino acid sequence having at least 70% sequence identity to SEQ ID NO: 1, 2, 3, 4, 6, 7, 8, 9, 10, 11, 12, 13, 14, or 15, wherein the peptide has a length of at least 10 amino acid residues.

11. The composition of claim 10, wherein the composition further comprise an agent that induces expression of the peptide.

12. The composition of claim 11, wherein the agent comprises a polynucleotide.

13. The composition of claim 12, wherein the polynucleotide comprises human FAIM-S mRNA, human FAIM-L mRNA, or a combination thereof.

14. The composition of claim 10, wherein the composition further comprise a clearing agent.

15. The composition of claim 14, wherein the clearing agent comprises an antibody that can target an aggregated protein.

16. The composition of claim 15, wherein the antibody comprises donanemab (Lilly), solanezumab (Lilly), gantenerumab (Roche), or a combination of two or more thereof.

17. The composition of claim 10, wherein the amino acid sequence has at least 90% sequence identity to SEQ ID NO: 1, 2, 3, 4, 6, 7, 8, 9, 10, 11, 12, 13, 14, or 15.

18. The composition of claim 10, wherein the amino acid sequence has at least 95% sequence identity to SEQ ID NO: 1, 2, 3, 4, 6, 7, 8, 9, 10, 11, 12, 13, 14, or 15.

19. The composition of claim 10, wherein the comprises the peptide or mimetic thereof comprising amino acid sequence having at least 70% sequence identity to SEQ ID NO: 1, 2, or 3.

20. The composition of claim 10, wherein the composition comprise about about 0.5 µM to about 50 µM of the peptide.

21. The composition of claim 10, wherein the composition comprise about 1.5 µM to about 20 µM of the peptide.

22. A method for treating a neurodegenerative or other proteinopathy in a subject in need thereof, the method comprising, administering a therapeutically effective amount of the composition of claim 10 to the subject in need thereof.

23. The method of claim 22, wherein the neurodegenerative or other proteinopathy comprises Alzheimer’s disease, Parkinson’s disease, Huntington’s disease, amyotropic lateral sclerosis, multiple tauopathies, spongiform encephalopathies, familial amyloidotic polyneuropathy, chronic traumatic encephalopathy, or a combination of two or more thereof.


SEQUENCE LISTING (78 sequences)

SEQ ID NO: 1 - protein, 179 aa, Homo sapiens [sequence omitted]

SEQ ID NO: 2 - protein, 201 aa, Homo sapiens
MASGDDSPIFEDDESPPYSLEKMTDLVAVWDVALSDGVHKIEFEHGTTSGKRVVYVDGKEEIRKEWMFKLVGKETFYVGAAKTKATINIDAISGFAYEYTLEINGKSLKKYMEDRSKTTNTWVLHMDGENFRIVLEKDAMDVWCNGKKLETAGEFVDDGTETHFSIGNHDCYIKAVSSGKRKEGIIHTLIVDNREIPEIAS

SEQ ID NO: 3 - protein, 213 aa, Homo sapiens
MLLPFIRTLPLLCYNHLLVSPDSATLSPPYSLEKMTDLVAVWDVALSDGVHKIEFEHGTTSGKRVVYVDGKEEIRKEWMFKLVGKETFYVGAAKTKATINIDAISGFAYEYTLEINGKSLKKYMEDRSKTTNTWVLHMDGENFRIVLEKDAMDVWCNGKKLETAGEFVDDGTETHFSIGNHDCYIKAVSSGKRKEGIIHTLIVDNREIPEIAS

SEQ ID NO: 4 - protein, 179 aa, Mus musculus
MTDLVAVWDVALSDGVHKIEFEHGTTSGKRVVYVDGKEEIRREWMFKLVGKETFFVGAAKTKATINIDAISGFAYEYTLEIDGKSLKKYMENRSKTTSTWVLRLDGEDLRVVLEKDTMDVWCNGQKMETAGEFVDDGTETHFSVGNHGCYIKAVSSGKRKEGIIHTLIVDNREIPELTQ

SEQ ID NO: 5 - protein, 20 aa, synthetic construct
CYIKAVSSRKRKEGIIHTLI

SEQ ID NO: 6 - protein, 90 aa, synthetic construct
MEDRSKTTNTWVLHMDGENFRIVLEKDTMDVWCNGKKLETAGEFVDDGTETHFSIGNHDCYIKAVSSGKRKEGIIHTLIVDNREIPEIAS

SEQ ID NO: 7 - protein, 11 aa, synthetic construct
MEDRSKTTNTW

SEQ ID NO: 8 - protein, 10 aa, synthetic construct
VLHMDGENFR

SEQ ID NO: 9 - protein, 10 aa, synthetic construct
IVLEKDTMDV

SEQ ID NO: 10 - protein, 10 aa, synthetic construct
WCNGKKLETA

SEQ ID NO: 11 - protein, 10 aa, synthetic construct
GEFVDDGTET

SEQ ID NO: 12 - protein, 10 aa, synthetic construct
HFSIGNHDCY

SEQ ID NO: 13 - protein, 10 aa, synthetic construct
IKAVSSGKRK

SEQ ID NO: 14 - protein, 10 aa, synthetic construct
EGIIHTLIVD

SEQ ID NO: 15 - protein, 9 aa, synthetic construct
NREIPEIAS

SEQ ID NO: 16 - protein, 6 aa, synthetic construct
HHHHHH

SEQ ID NO: 17 - DNA, 1090 bp, Homo sapiens
  source 1..1090: mol_type = other DNA
tgggtccgtggcggcgggaggggtggcctcctgcgctggtcgccccaggggacctgagaggcgcgacaaacagtcggcgcgtttggtactcgcgcctgcagagctttcaacctccgcgccggctgcgcctgtttctcggccaggggagcaaggccacgcggcctacgcagccgagtcggaaccaaccggttgtttggtgaaacctaccccagagcctcccgcggcccacagagcacagccctccttacagcctagaaaaaatgacagatctcgtagctgtttgggatgttgctttaagtgacggagtccacaagatcgaatttgaacatgggactacatcaggcaaacgagtagtatatgtagatggaaaggaagagataagaaaagagtggatgttcaaattagtgggcaaagaaacattctatgttggagctgcaaagacaaaagcgaccataaatatagacgctatcagtggttttgcttatgaatatactctggaaattaatgggaaaagtctcaagaagtatatggaggacagatcaaaaaccaccaatacttgggtattacacatggatggtgagaactttagaattgttttggaaaaagatgctatggacgtatggtgcaatggtaaaaaattggagacagcgggtgagtttgtagatgatgggactgaaactcacttcagtatcgggaaccatgactgttacataaaggctgtcagtagtgggaagcggaaagaagggattattcatactctcattgtggataatagagaaatcccagagattgcaagttaatgaattttcatcttaagaagtaaagatcaggactttttaattactgtggtaattaaatgtgttcagtatgtacttatcagtacatttagtctgcaatgttttaattttttaaaaagttacatgaaactaacattccaagggtcaggaaaaaaaccaattatgtatagtcataaaaattacaatttatgatgcaaataatgtaaaatgttttaaagacaaatggcaaataagatatggaccaaagtcactaatgttttacaacagtaacctttactataataaatactttt

SEQ ID NO: 18 - DNA, 1150 bp, Homo sapiens
  source 1..1150: mol_type = other DNA
tgggtccgtggcggcgggaggggtggcctcctgcgctggtcgccccaggggacctgagaggcgcgacaaacagtcggcgcgtttggtactcgcgcctgcagagctttcaacctccgcgccggctgcgcctgtttctcggccaggggagcaaggccacgcggcctacgcagccgagtcggaaccaaccggttgtttggtgaaacctaccccagagcctcccgcggcccacagagcacagactgtttttgccaaccatggcatctggagatgacagtcctatctttgaagatgatgaaagccctccttacagcctagaaaaaatgacagatctcgtagctgtttgggatgttgctttaagtgacggagtccacaagatcgaatttgaacatgggactacatcaggcaaacgagtagtatatgtagatggaaaggaagagataagaaaagagtggatgttcaaattagtgggcaaagaaacattctatgttggagctgcaaagacaaaagcgaccataaatatagacgctatcagtggttttgcttatgaatatactctggaaattaatgggaaaagtctcaagaagtatatggaggacagatcaaaaaccaccaatacttgggtattacacatggatggtgagaactttagaattgttttggaaaaagatgctatggacgtatggtgcaatggtaaaaaattggagacagcgggtgagtttgtagatgatgggactgaaactcacttcagtatcgggaaccatgactgttacataaaggctgtcagtagtgggaagcggaaagaagggattattcatactctcattgtggataatagagaaatcccagagattgcaagttaatgaattttcatcttaagaagtaaagatcaggactttttaattactgtggtaattaaatgtgttcagtatgtacttatcagtacatttagtctgcaatgttttaattttttaaaaagttacatgaaactaacattccaagggtcaggaaaaaaaccaattatgtatagtcataaaaattacaatttatgatgcaaataatgtaaaatgttttaaagacaaatggcaaataagatatggaccaaagtcactaatgttttacaacagtaacctttactataataaatactttt

SEQ ID NO: 19 - protein, 8 aa, synthetic construct
DYKDDDDK

SEQ ID NO: 20 - DNA, 25 bp, synthetic construct
  source 1..25: mol_type = other DNA
aatatggcatctggagatgacagtc

SEQ ID NO: 21 - DNA, 24 bp, synthetic construct
  source 1..24: mol_type = other DNA
ttaacttgcaatctctgggatttc

SEQ ID NO: 22 - DNA, 28 bp, synthetic construct
  source 1..28: mol_type = other DNA
aatatgacagatctcgtagctgtttggg

SEQ ID NO: 23 - DNA, 35 bp, synthetic construct
  source 1..35: mol_type = other DNA
atatagaattcatatggcatctggagatgacagtc

SEQ ID NO: 24 - DNA, 35 bp, synthetic construct
  source 1..35: mol_type = other DNA
tatatctcgagttaacttgcaatctctgggatttc

SEQ ID NO: 25 - DNA, 38 bp, synthetic construct
  source 1..38: mol_type = other DNA
atatagaattcatatgacagatctcgtagctgtttggg

SEQ ID NO: 26 - DNA, 37 bp, synthetic construct
  source 1..37: mol_type = other DNA
atatagaattctaatgacagatctcgtagctgtttgg

SEQ ID NO: 27 - DNA, 30 bp, synthetic construct
  source 1..30: mol_type = other DNA
atggtaccacttgcaatctctgggatttct

SEQ ID NO: 28 - DNA, 38 bp, synthetic construct
  source 1..38: mol_type = other DNA
atatagaattctaatggcatctggagatgacagtccta

SEQ ID NO: 29 - DNA, 24 bp, synthetic construct
  source 1..24: mol_type = other DNA
atgacagatctcgtagctgtttgg

SEQ ID NO: 30 - DNA, 22 bp, synthetic construct
  source 1..22: mol_type = other DNA
atggcatctggagatgacagtc

SEQ ID NO: 31 - DNA, 23 bp, synthetic construct
  source 1..23: mol_type = other DNA
atgaccgagcgccgcgtcccctt

SEQ ID NO: 32 - DNA, 25 bp, synthetic construct
  source 1..25: mol_type = other DNA
ttacttggcggcagtctcatcggat

SEQ ID NO: 33 - DNA, 20 bp, synthetic construct
  source 1..20: mol_type = other DNA
atggacatcgccatccacca

SEQ ID NO: 34 - DNA, 21 bp, synthetic construct
  source 1..21: mol_type = other DNA
ctatttcttgggggctgcggt

SEQ ID NO: 35 - DNA, 26 bp, synthetic construct
  source 1..26: mol_type = other DNA
atggatgtattcatgaaaggactttc

SEQ ID NO: 36 - DNA, 23 bp, synthetic construct
  source 1..23: mol_type = other DNA
ttaggcttcaggttcgtagtctt

SEQ ID NO: 37 - DNA, 20 bp, synthetic construct
  source 1..20: mol_type = other DNA
atggcgacgaaggccgtgtg

SEQ ID NO: 38 - DNA, 23 bp, synthetic construct
  source 1..23: mol_type = other DNA
ttattgggcgatcccaattacac

SEQ ID NO: 39 - DNA, 28 bp, synthetic construct
  source 1..28: mol_type = other DNA
caccgtgacggatctcgtagctgtttgg

SEQ ID NO: 40 - DNA, 25 bp, synthetic construct
  source 1..25: mol_type = other DNA
aaacaacagctacgagatccgtcac

SEQ ID NO: 41 - DNA, 27 bp, synthetic construct
  source 1..27: mol_type = other DNA
caccgacagatctcgtagctgtttggg

SEQ ID NO: 42 - DNA, 24 bp, synthetic construct
  source 1..24: mol_type = other DNA
aaacaaacagctacgagatctgtc

SEQ ID NO: 43 - DNA, 32 bp, synthetic construct
  source 1..32: mol_type = other DNA
tggactatcatatgcttaccgtaacttgaaag

SEQ ID NO: 44 - DNA, 25 bp, synthetic construct
  source 1..25: mol_type = other DNA
acggatctcgtagctgtttgggacg

SEQ ID NO: 45 - DNA, 25 bp, synthetic construct
  source 1..25: mol_type = other DNA
ccagcgtgtactcgtatgcgaagcc

SEQ ID NO: 46 - DNA, 22 bp, synthetic construct
  source 1..22: mol_type = other DNA
cagaagaactcgtcaagaaggc

SEQ ID NO: 47 - DNA, 23 bp, synthetic construct
  source 1..23: mol_type = other DNA
caagcgaaacatcgcatcgagcg

SEQ ID NO: 48 - DNA, 22 bp, synthetic construct
  source 1..22: mol_type = other DNA
tgggtattacacatggatggtg

SEQ ID NO: 49 - DNA, 20 bp, synthetic construct
  source 1..20: mol_type = other DNA
acaaactcacccgctgtctc

SEQ ID NO: 50 - DNA, 20 bp, synthetic construct
  source 1..20: mol_type = other DNA
cagctggtttgacactggac

SEQ ID NO: 51 - DNA, 20 bp, synthetic construct
  source 1..20: mol_type = other DNA
ggcgctcttcatgttttcca

SEQ ID NO: 52 - DNA, 20 bp, synthetic construct
  source 1..20: mol_type = other DNA
acatgaagcactggcctttc

SEQ ID NO: 53 - DNA, 22 bp, synthetic construct
  source 1..22: mol_type = other DNA
tctccttcatcttggtcagcac

SEQ ID NO: 54 - DNA, 20 bp, synthetic construct
  source 1..20: mol_type = other DNA
tggcagcaaagaaacacctg

SEQ ID NO: 55 - DNA, 22 bp, synthetic construct
  source 1..22: mol_type = other DNA
caggagcgcagtttcataaagc

SEQ ID NO: 56 - DNA, 20 bp, synthetic construct
  source 1..20: mol_type = other DNA
ctgacttcaacagcgacacc

SEQ ID NO: 57 - DNA, 20 bp, synthetic construct
  source 1..20: mol_type = other DNA
gtggtccaggggtcttactc

SEQ ID NO: 58 - DNA, 29 bp, synthetic construct
  source 1..29: mol_type = other DNA
caaacgagtagtatatggagatggaaagg

SEQ ID NO: 59 - DNA, 25 bp, synthetic construct
  source 1..25: mol_type = other DNA
cctgatgtagtcccatgttcaaatt

SEQ ID NO: 60 - DNA, 29 bp, synthetic construct
  source 1..29: mol_type = other DNA
aaaagcgaccataaatggagacgctatca

SEQ ID NO: 61 - DNA, 25 bp, synthetic construct
  source 1..25: mol_type = other DNA
gtctttgcagctccaacatagaatg

SEQ ID NO: 62 - DNA, 24 bp, synthetic construct
  source 1..24: mol_type = other DNA
atgacagatctcgtagctgtttgg

SEQ ID NO: 63 - DNA, 24 bp, synthetic construct
  source 1..24: mol_type = other DNA
ttaacttgcaatctctgggatttc

SEQ ID NO: 64 - DNA, 20 bp, synthetic construct
  source 1..20: mol_type = other DNA
atggcgacgaaggccgtgtg

SEQ ID NO: 65 - DNA, 32 bp, synthetic construct; identical to SEQ ID NO: 43
  source 1..32: mol_type = other DNA

SEQ ID NO: 66 - DNA, 19 bp, synthetic construct
  source 1..19: mol_type = other DNA
tatggctagcatgactggt

SEQ ID NO: 67 - protein, 179 aa, Homo sapiens
MTDLVAVWDVALSDGVHKIEFEHGTTSGKRVVYVDGKEEIRKEWMFKLVGKETFYVGAAKTKATINIDAISGFAYEYTLEINGKSLKKYMEDRSKTTNTWVLHMDGENFRIVLEKDAMDVWCNGKKLETAGEFVDDGTETHFSIGNHDCYIKAVSSGKRKEGIIHTLIVDNREIPEIAS

SEQ ID NO: 68 - protein, 179 aa, Macaca mulatta
MTDLVAVWDVALSDGVHKIEFEHGTTSGKRVVYVDGKEEIRKDWMFKLVGRETFCVGAAKTKATINIDAISGFAYEYTLEINGKSLKKYMENRSKTTNTWVLHMDGENFRIVLEKDTMDVWCNGKKLETAGEFVDDGTETHFSLGNHDCYIKAVSSGKRKEGIIHTLIVDNREIPEIAS

SEQ ID NO: 69 - protein, 179 aa, Rattus norvegicus
MTDLVAVWDVALSDGVHKIEFEHGTTSGKRVVYVDGKEEIRREWMFKLVGKETFFVGAAKTKATINIDAISGFAYEYTLEIDGKSLKKYMENRSKTTNTWVLHLDGEELRVVLEKDTMDVWCNGQRMETAGEFVDDGTETHFSVGNHDCYIKAVSSGKRREGIIHTLIVDNREIPELTQ

SEQ ID NO: 70 - protein, 179 aa, Mus musculus; identical to SEQ ID NO: 4

SEQ ID NO: 71 - protein, 202 aa, Gallus gallus
MASREEVTLSEEEVRSRYSHLEKMTDLVAVWEVALSDGVHKIEFEHGTTSGKRVVYVDGKEIIRKEWMFKLVGKETFTVGVSKTRATINIDAVSGFAYEYTLEINGKSLKKYMENRSKTTNTWVLSLGGTDYRVVLEKDTMDVWCNGEKMETAGEFVEDGTETHFSVGDHSCYIKAVSSGKRKEGIIHTLIVDDREIPEAVE

SEQ ID NO: 72 - protein, 179 aa, Danio rerio
MSGDLVAVWEVALSDGVHRIEFEHGTTTGKRVIYIDGKEVIRRDWMFKLVGKETFNVGSTDTKATINIDAVSGFAYEYTLEINGQSLRKYMENRSKVTSTWLLNLDGIDCRVVLEKDTMDIWCNGQKMETAGEFVEDGTETHFTLGDHDCCIKAVSSGKRRDGIIHTLLVDGMEISEAE

SEQ ID NO: 73 - protein, 190 aa, Caenorhabditis elegans
MPEGDVVATWNVPLHAQVHKIEFEHGTTTGKRVIRIDGNEILRRDWMFKLVGKEAFKVGDMKCIINVEALGTFAYEYSLDVNGKTFNKFKEEQNKRLHSWETTLSGHQWRVVLDKESMEIWANGNNIDTAGEFVDNGTVTHFELGKNPCKIIAQSSGKKKTGVTHTLYVNGEIVPSTVDKPNPSNQPGSS

SEQ ID NO: 74 - protein, 202 aa, unidentified
MASRDEITITQEDMARYAHADRMADLVAMWEVALSDGVHKIEFEHGTTSGKRVVYVDGKELIRRDWMFKLVGKETFTVGATETKATINIDAVSGFAYEYTLEINGKSLKKFIENRSKTSKTWVLQLDGVDIRVVLEKDTMDIWCNGDKMETAGEFVEEGTETHFTLLDRHECCIKAVSSGRRREGIVHSLILNGVEVPEVLE

SEQ ID NO: 75 - protein, 228 aa, Drosophila melanogaster
MSFLSPTLRLENLSTQPTMTQDHVPEDQRYNKQNIVAQWCVPINGKMYRIELEHGTTSGRRMIWVNGREVLRRDWMFKLVGEDTFHIDQTRCIIRVDPAPGFKYEYSLYIDGKSHDQYTEDMTRQYRLWLYTDDAAADAPQEYRIMLKLDTLSLYVNDELRTEESVFVHGGTDTKFLLQDTEFVLQARSSGNKHDGIVHTLLANGVAVPEAKIQEIMQEPVSILQSSN

SEQ ID NO: 76 - protein, 213 aa, Anopheles gambiae
FYTLLPSTMLNNADILSNIPPLGGKEGAAGPHREVVARWRVPMYGKVYEIEFEHGTASGKRVLWIDKQEVFRRDWMFKLVGEDMFKLEDKRCIIRVDPMPGFRYSYSLFVDGKSYEQFTESQAKALKTWEAKLGDNFYRIVLEKNTLNIYVNGKLIEENGEFVDGGTDTTFLEDGNTFVLSARTSGNKREGIVHRLTVNGAEVFDAGGATTAL

SEQ ID NO: 77 - protein, 182 aa, Suberites domuncula
MANPGDIAACWDIKLADGPHKVLLEHGTTSGKRVIFVDGVEVLRRNWLFRLVGSESFKIGKRAAEIEIISGNLTSFEYVLKIDGKTLKKFIESQVKNTRAWLPNVSGAAHRVVLDIETMDVYVDGEKSDTFGEFTESGVETHFTMSGFSVHISSLSSGNKRKGLLYHLHVDGTKLEPVNDLQ

SEQ ID NO: 78 - protein, 253 aa, unidentified
MLFAKFLQQAEKPRERKQGGRSRSEVVLWSCWFGCERARWKLASKRVIGVTVTMSTPKGSEHVLTGGQMRWTCRLADGEHVIEFTHGTTSGKRVITVDGKEVVRKNWLFKLVGEEPFEFGPAGDKHKAKITVVAAGMRYRYTLYVDNKPLKEFSDTRTRSTKTWFFPVAGQNHLVVMDTETMEIWADGSKVEAVGEFVEDGTETHLEVGGQPAYVLTTKSGERGKLEHILFLNDEEVLPTEEDRYSGVRWSAS